(19) United States
(12) Patent Application Publication
(10) Pub. No.: US 2014/0088045 A1
(43) Pub. Date: Mar. 27, 2014

(54) PRODUCT COMPRISING A NICOTINE-CONTAINING MATERIAL AND AN ANTI-CANCER AGENT

(71) Applicants: Basil Rigas, Setauket, NY (US); Jason B. Rigas, Setauket, NY (US)

(72) Inventors: Basil Rigas, Setauket, NY (US); Jason B. Rigas, Setauket, NY (US)

(21) Appl. No.: 14/013,019

(22) Filed: Aug. 28, 2013

Related U.S. Application Data
(60) Provisional application No. 61/703,999, filed on Sep. 21, 2012.

(51) Int. Cl. A61K 31/661; A61K 45/06; A24F 47/00; A61K 31/12; A61M 15/06; A61K 31/465; A61K 31/675

(52) U.S. Cl. CPC A61K 31/661; A61K 31/465; A61K 45/06; A61K 31/675; A61K 31/12; A61M 15/06; A24F 47/008
USPC 514/81; 514/120; 514/119; 128/202.21; 206/528

(57) ABSTRACT

The present invention provides a composition comprising a nicotine-containing material and an anti-cancer agent usable in the treatment and/or prevention or reduction of the risk of cancer and precancerous conditions as well as for preventing or reducing the risk of cancer recurrence. Furthermore, a composition comprising a nicotine-containing material and an anti-inflammatory agent usable in the treatment and/or prevention or reduction of the risk of inflammation, is provided. The nicotine containing composition can also include both an anti-cancer agent and an anti-inflammatory agent A device for administering the composition of the present invention to subjects can be a cigarette, smoking pipe, smokeless tobacco, electronic cigarette, transdermal patch or the like.

### CROSS REFERENCE TO RELATED APPLICATION

This application claims priority on, and incorporates by reference, U.S. Ser. No. 61/703,999 filed Sep. 21, 2012.

### BACKGROUND OF THE INVENTION

Consumption of nicotine-containing products, in particular smoking, is known to affect health and to increase the risk of developing cancer. In particular, the risk of developing lung cancer among smokers is significantly higher than among non-smokers. Lung cancer is the major cause of cancer mortality in the industrial world and its association with smoking is firmly established.

Consumption of tobacco products, in particular smoking, has also been linked to an increased risk of multiple cancers, besides the prototypical case of lung cancer. A detailed analysis of the epidemiological evidence on the association between tobacco smoking and cancer concluded that there is sufficient evidence to establish a causal association between cigarette smoking and cancer of the nasal cavities and paranasal sinuses, nasopharynx, stomach, liver, kidney (renal cell carcinoma) and uterine cervix, and for adenocarcinoma of the oesophagus and myeloid leukaemia (A. J. Sasco et al. Lung Cancer 2004 August 45, Suppl 2, S3-9). These findings add to the previously established list of cancers causally associated with cigarette smoking, namely cancer of the lung, oral cavity, pharynx, larynx, oesophagus, pancreas, urinary bladder and renal pelvis. Other forms of tobacco smoking, such as cigars, pipes and bidis, also increase risk for cancer, including cancer of the lung and parts of the upper aerodigestive tract. Smoking is currently responsible for a third of all cancer deaths in many Western countries. It has been estimated that one in every two smokers will be killed by smoking.

Furthermore, brain cancers such as glioma are also associated with smoking while primary lung cancers are also known to spread to the brain. At least 40% of patients with lung cancer develop brain metastases at some point during their disease. In particular, small cell lung cancer can spread to the brain rapidly, often before the diagnosis of lung cancer is made.

Despite significant advances in its early detection, the survival of lung cancer patients remains poor, with the 5-year survival being as low as 5%. Because of frequent and widespread metastases, surgical procedures for lung cancer are not particularly effective and chemotherapy, the treatment of choice in inoperable cases of lung cancer, has only limited efficacy. The prevention or reduction of the risk of cancer is currently the only viable option in controlling this dreadful disease.

Smoking cessation would be the most effective method to prevent lung cancer. However, even though it is widely known that smoking causes cancer, smoking is a very difficult addiction to break and currently-marketed smoking cessation products are of limited efficacy. Even patients diagnosed with lung or brain cancer or with precancerous conditions thereof often fail to quit smoking. Thus, there is a pressing need to develop new methods to prevent cancers such as lung and brain cancer in individuals at increased risk of developing these cancers, in particular smokers.

Chemoprevention or reduction of the risk of cancer, an emerging highly promising approach to cancer prevention or reduction of the risk of, is defined as the administration of an anti-cancer agent, which can comprise a synthetic or a natural compound, to individuals at risk of developing cancer to prevent or to reduce the risk of developing cancer or to those who already had cancer to prevent or to reduce the risk of its recurrence.

### SUMMARY OF THE INVENTION

The inventor has found that anti-cancer agents can be advantageously employed for the prevention or reduction of the risk of cancers, for instance lung and brain cancers and precancerous conditions thereof, when these anti-cancer agents are administered in combination with smoking and/or with another nicotine-containing material described herein. This administration is highly suitable for individuals consuming tobacco products, in particular for smokers. Combining the administration of an anti-cancer agent, particularly an agent preventing or reducing the risk of lung cancer, with tobacco products including smoking or with smoking cessation products, for instance with nicotine chewing gum, would be very efficient in the prevention or reduction of the risk of lung cancer or brain cancer or other smoking/tobacco related cancers.

The combination of smoking with the anti-cancer agent a) maximizes the efficacy of the anti-cancer agent, since the anti-cancer agent will be present when the tobacco carcinogens are inhaled, and b) provides an improved or close to absolute (100%) individual compliance in terms of the intake of the anti-cancer agent (which will increase anti-cancer efficacy). Poor compliance with the intake of prescribed medications is well established, especially for long-term administration of agents preventing or reducing the risk of disease. A case in point is the non-adherence to the use of aspirin among patients post myocardial infarction and other coronary events in which aspirin was prescribed to prevent another myocardial infarction (E Shantsila, G. Y. H. Lip, Journal of Translational Medicine 2008, 6, 47).

The present invention relates to a product comprising a nicotine-containing material and an anti-cancer agent. The anti-cancer agent may be a compound of natural or synthetic origin. The anti-cancer agent is capable of either preventing or reducing the risk of or treating cancer or combination thereof.

In one embodiment, the anti-cancer agent in the product of the present invention comprises a phospho-nonsteroidal anti-inflammatory agent (NSAID) having covalently attached a phosphate moiety (phospho-NSAIDs) through a linker moiety. Examples of phospho-NSAIDs include, but are not limited to, compounds selected from the group consisting of:

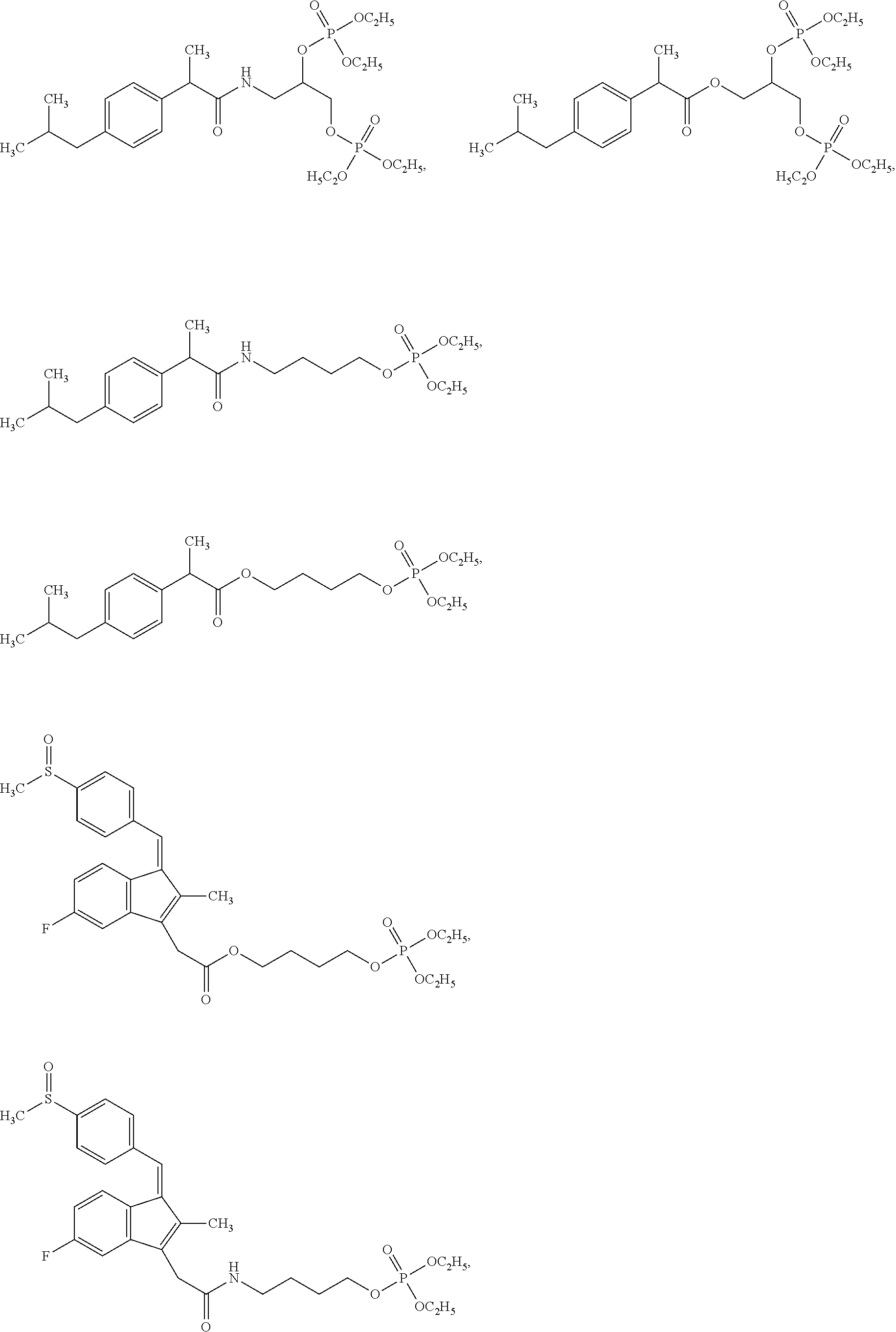

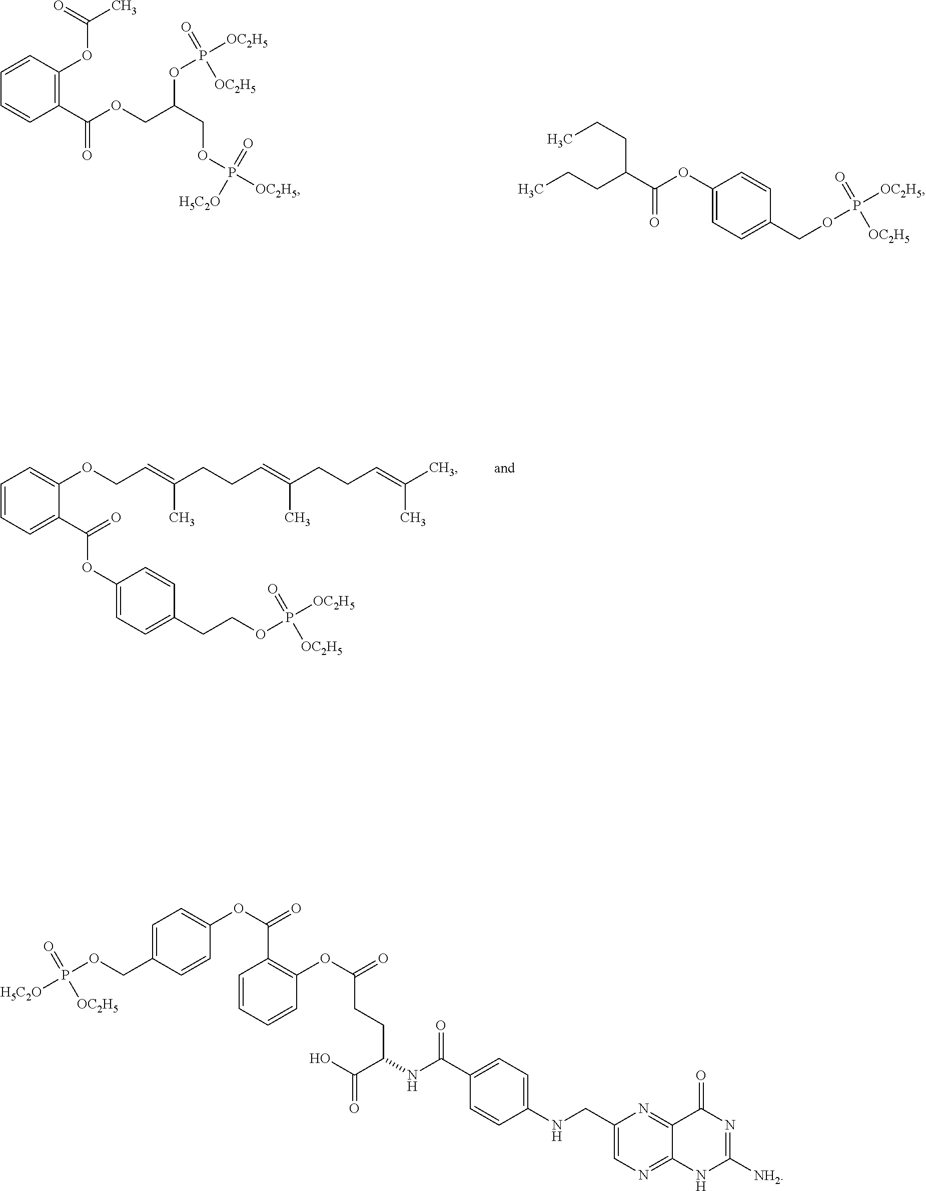

In a further embodiment, the anti-cancer agent in the product of the present invention is an oxidative stress enhancer.

In a further embodiment, the anti-cancer agent may comprise a compound of natural origin such as, for instance, curcumin or other curcuminoids.

In some embodiments of the present invention, the anti-cancer agent comprises one single compound having anti-cancer activity, whereby it is preferred that the anti-cancer agent essentially consists of said compound. In yet other embodiments of the invention, the anti-cancer agent comprises a combination of at least two different compounds having anti-cancer activity. Accordingly, the product of the present invention may comprise a combination of at least two different compounds having anti-cancer activity, for instance a combination of a phospho-NSAID and curcumin.

In some preferred embodiments, the nicotine-containing material in the product of the present invention is tobacco leaf.

Preferably, the product of the present invention contains nicotine and the anti-cancer agent in a preferred ratio of from 1000:1 to 1:10 (wt:wt).

The product of the present invention may be a device capable of delivering both the tobacco product and the anti-cancer agent that can be selected from, but not limited to, the group consisting of cigarette, cigar and smoking pipe, whereby the smoking device may optionally include an additional unit which renders the anti-cancer agent suitable for inhalation. However, the product of the present invention may also be a novel device capable of delivering the tobacco product and the anti-cancer agent.

Alternatively, the product may be a smoking cessation product. Accordingly, the product may be a transdermal patch, an inhalation device, a rectal suppository or an orally applied product.

In still a further embodiment, the product is a smokeless tobacco product.

A further aspect of the invention relates to an anti-cancer agent for use in the prevention or reduction of the risk of and/or treatment of cancer and/or precancerous conditions, wherein said anti-cancer agent is administered simultaneously with nicotine. The cancer may, for instance, be a lung cancer, brain cancer or a precancerous condition thereof.

In a preferred embodiment, the anti-cancer agent is inhaled together with tobacco smoke.

### BRIEF DESCRIPTION OF THE FIGURES

FIG. 1A-1G are images of smoking devices comprising a nicotine-containing material and an anti-cancer agent.

FIG. 2 is a graph showing the levels of phospho-sulindac (PS V) and its metabolites in the lungs (A) and plasma (B) of mice subjected to aerosol administration of PS V.

FIG. 3 is a graph showing the survival rates of control and aerosolized-PS treated groups of mice implanted orthotopically with A549 cells.

FIG. 4 is an image showing that aerosol administration of PS V prevents lung tumorigenesis.

FIG. 5 is a graph showing that aerosol administration of PS V prevents lung tumorigenesis.

FIG. 6 is a graph showing the lung level of PS V after inhalation and oral administration.

FIG. 7 is a graph showing the plasma level of PS V after inhalation and oral administration.

FIG. 8 is a graph showing the apparatuses used to assess experimentally the aerosolization of anticancer agents.

FIG. 9 shows chromatograms from aerosolized anticancer agents.

FIG. 10 shows the inhibition of NF-κB by PS V and the effect of PS V and erlotinib on lung cancer growth.

FIG. 11A is a cross-sectional view of a cigarette holder with a replaceable cartridge containing capsules of an agent.

FIG. 11B is a cross-sectional view of a cigarette holder similar to that of FIG. 11A, but with a battery having a central bore for pass through of smoke and vapor.

FIG. 11C is a cross-sectional view of a cigarette holder similar to that of FIG. 11A, but with three batteries placed in a manner that allows the free flow of smoke and vapor.

FIG. 12A is a cross-sectional view of the device of FIG. 11A showing a single battery and air passage in the lower region.

FIG. 12B is a cross-sectional view of the device of FIG. 11B showing a hollow core battery to provide a central air passage.

FIG. 12C is a cross-sectional view of the device of FIG. 11C with three batteries, to provide multiple air passages.

FIG. 13A is an elevational view of a cartridge usable in the device of FIGS. 11A-11C, containing capsules of anti-inflammatory or anti-cancer agents.

FIG. 13B is a side cross-section of the cartridge of FIG. 13A used to dispense tobacco smoke and vaporized drug.

FIG. 14 is a cross-sectional view of a pipe adapted to dispense smoke from burning tobacco and vaporized drug.

### DETAILED DESCRIPTION OF THE PREFERRED EMBODIMENT

One or more embodiments of the invention will be described by way of example and not limitation, and the invention is not limited to the embodiment(s). The present invention provides a novel product or composition comprising a nicotine-containing material and an anti-cancer agent.

As used herein the term “anti-cancer agent” refers to a natural or synthetic agent that is capable of either preventing or reducing the risk of or treating cancer or both. Preferably, the anti-cancer agent is capable of inhibiting the proliferation or preventing or reducing the risk of the development of cancer cells.

As used herein, the term “anti-inflammatory agent” refers to a natural or synthetic agent that is capable of either preventing or reducing the risk of or treating an inflammatory disease (or condition), or both.

In some embodiments of the present invention, the anti-cancer agent comprises a combination of at least two different compounds having anti-cancer activity. Accordingly, the product of the present invention may comprise a combination of at least two different compounds having anti-cancer activity. For instance, the product may contain two different compounds having anti-cancer activity in the ratio of from 10:1 to 1:10, more preferred from 7:1 to 1:7, particularly preferred from 4:1 to 1:4, for instance 1:1 (weight:weight). As an example, a combination of curcumin and a phospho-NSAID can be mentioned.

Preferred anti-cancer agents are capable of inhibiting the growth or preventing or reducing the risk of the development of solid tumors in vivo. Preferred anti-cancer agents are also capable of reducing the size of a solid tumor in vivo.

The anti-cancer agent may comprise compounds including, but not being limited to, androgen inhibitors, such as flutamide and luprolide; antiestrogens, such as tamoxifen; antimetabolites and cytotoxic agents, such as daunorubicin, fluorouracil, floxuridine, interferon alpha, methotrexate, plicamycin, mercaptopurine, thioguanine, adriamycin, carmustine, lomustine, cytarabine, cyclophosphamide, doxorubicin, estramustine, altretamine, hydroxyurea, ifosfamide, procarbazine, mutamycin, busulfan, mitoxantrone, streptozocin, bleomycin, dactinomycin, and idamycin; hormones, such as medroxyprogesterone, estramustine, ethinyl estradiol, estradiol, leuprolide, megestrol, octreotide, diethylstilbestrol, chlorotrianisene, etoposide, podophyllotoxin, and goserelin; nitrogen mustard derivatives, such as melphalan, chlorambucil, methlorethamine, and thiotepa, steroids, such as betamethasone; differentiation-inducing agents, such as retinoic acid, vitamin D, cytokines; and other antineoplastic agents, such as platinum compounds, dicarbazine, asparaginase, leucovorin, mitotane, vincristine, vinblastine, and taxanes (e.g., taxol, paclitaxel, docetaxel).

In one preferred embodiment, the anti-cancer agent comprises a tyrosine kinase inhibitor (TKI). A TKI inhibits the tyrosine kinase activity of at least one tyrosine kinase. The inhibition may be reversible or irreversible. TKIs include, but are not limited to, compounds such as imatinib, dasatinib, nilotinib, gefitinib, erlotinib, lapatinib, sunitinib, sorafenib and pazopanib. Various TKIs are, for instance, described in Hartmann et al. (J. Th. Hartman et al. Cur. Drug Metab, 2009, 10, pp. 470-481).

In the present invention, an “anticancer agent” means a compound effective to treat or prevent a proliferative disorder. Examples of anticancer agents are compounds that induce oxidative stress in the target cells or stromal cells of the proliferative disorder sensitive to the anti-cancer agent. Anticancer agents may comprise but are not limited to: a) androgen inhibitors, such as flutamide and luprolide; b) antiestrogens, such as tamoxifen; c) antimetabolites and cytotoxic agents, such as daunorubicin, fluorouracil, floxuridine, interferon alpha, methotrexate, plicamycin, mercaptopurine, thioguanine, adriamycin, carmustine, lomustine, cytarabine, cyclophosphamide, doxorubicin, estramustine, altretamine, hydroxyurea, ifosfamide, procarbazine, mutamycin, busulfan, mitoxantrone, streptozocin, bleomycin, dactinomycin, and idamycin; d) hormones, such as medroxyprogesterone, estramustine, ethinyl estradiol, estradiol, leuprolide, megestrol, octreotide, diethylstilbestrol, chlorotrianisene, etoposide, podophyllotoxin, and goserelin; e) nitrogen mustard derivatives, such as melphalan, chlorambucil, methlorethamine, and thiotepa, f) steroids, such as betamethasone, prednisone, prednisolone; g) differentiation-inducing agents, such as retinoic acid, vitamin D, cytokines; and g) other antineoplastic agents, such as platinum compounds, dicarbazine, asparaginase, leucovorin, mitotane, vincristine, vinblastine, and taxanes (e.g., taxol, paclitaxel, docetaxel), folic acid analogs and purine and pyrimidine analogs, protein tyrosine kinase inhibitors, immunomodulators, biological response modifiers, and monoclonal antibodies; h) natural products, such as, for example, vinca alkaloids, taxanes, and camptothecins; and i) Nonsteroidal anti-inflammatory drugs (NSAIDs) which are categorized as Salicylates, which include aspirin (acetylsalicylic acid), difusinal, salsalate; Propionic acid derivatives, which include ibuprofen, dexibuprofen, naproxen, fenoprofen, ketoprofen, dexketoprofen, flurbiprofen, oxaprozin, oxoprofen; Acetic acid derivatives, which include indomethacin, tolmetin, sulindac, etodolac, ketorolac, diclofenac, nambumetone; Enolic acid (Oxicam) derivatives, which include piroxicam, meloxicam, tenoxicam, droxicam, lornoxicam, isoxicam; Fenamic acid derivatives (Fenamates), which include mefenamic acid, meclofenamic acid, flufenamic acid, tolfenamic aci; Selective COX-2 inhibitors (Coxibs), which include celecoxib and paracetamol; Sulphonanilides, which include nimesulide; and Others, which include licofelone. A list of anti-cancer agents can be found in L. Brunton, B. Chabner and B. Knollman (eds). Goodman and Gilman's, The Pharmacological Basis of Therapeutics, Twelfth Edition, 2011, McGraw Hill Companies, New York, N.Y.

Further preferred anti-cancer agents for use in the present invention may comprise a compound such as difluoromethylornithine or erlotinide.

In one aspect, the anti-cancer agent in the present invention has a structure of Formula (I):

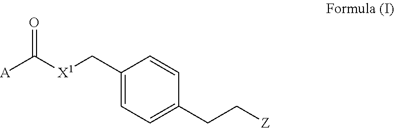

or an enantiomer, a diastereomer, a racemate, a tautomer, salt, hydrate, cocrystal, or compositions thereof.

In Formula I, A is an optionally substituted aliphatic, heteroaliphatic, aromatic, heteroaromatic substituent or alkylaryl substituent having 1 to 100 carbon atoms or is selected from:

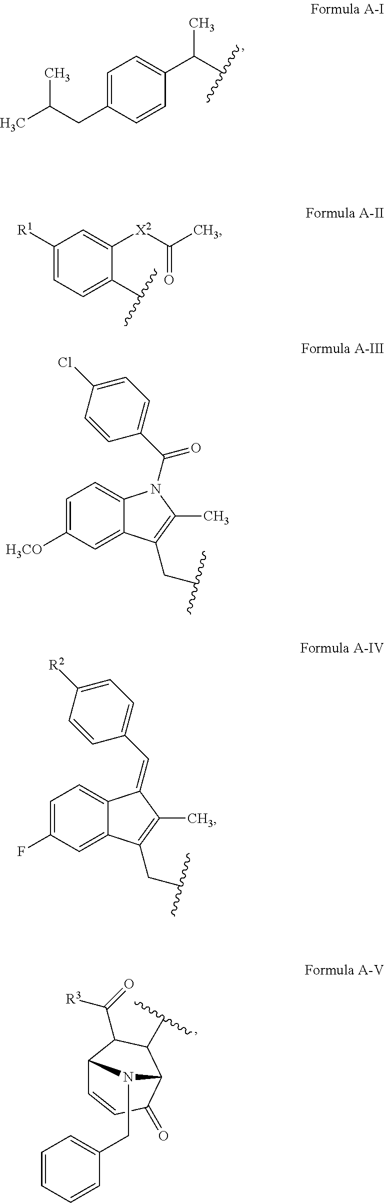

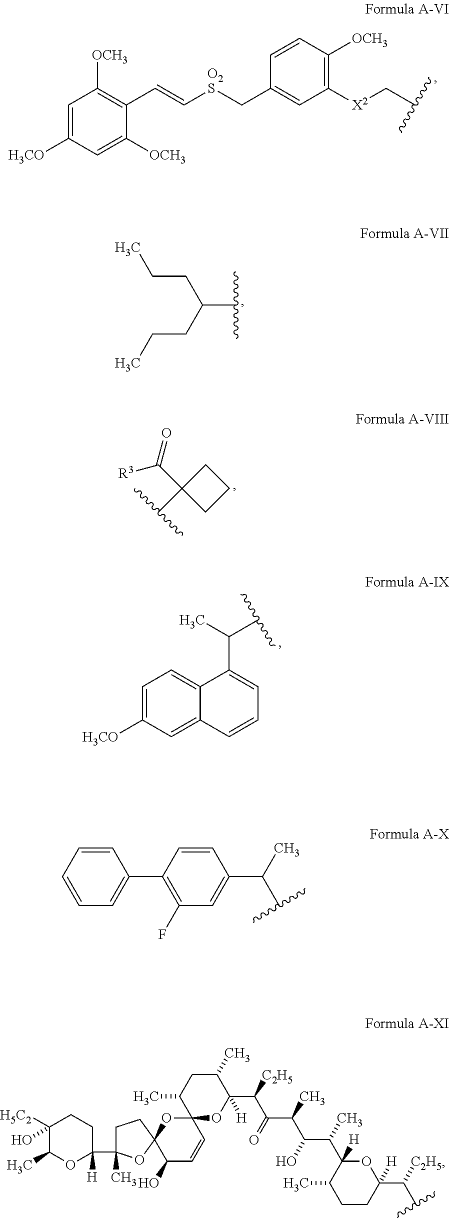

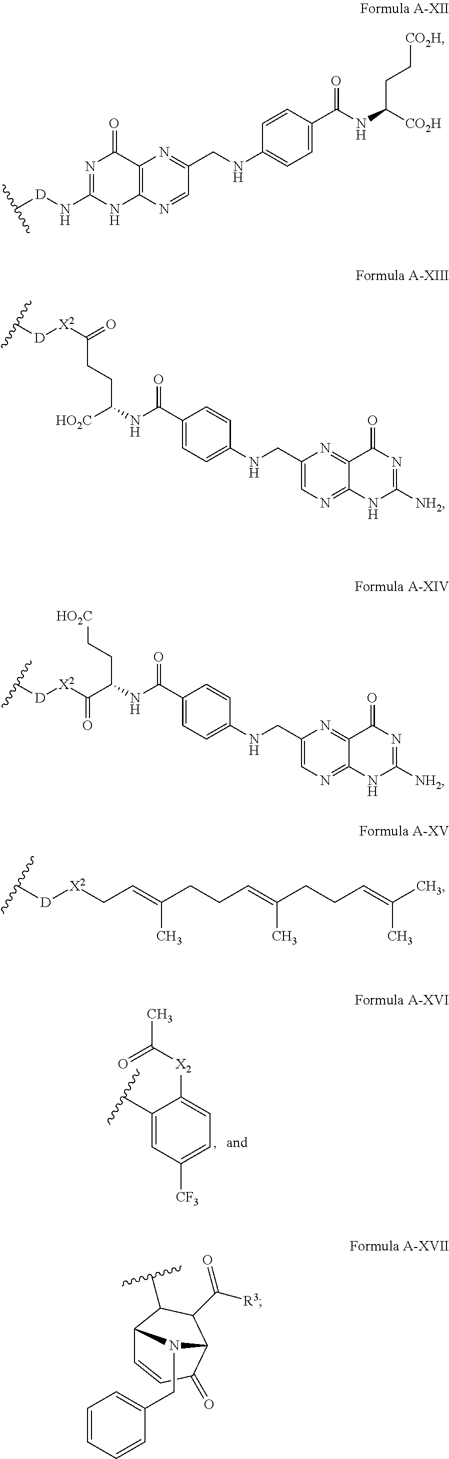

D is absent or

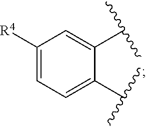

X¹ and X² are independently selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, —X¹—(CH₂)₄—Z, and

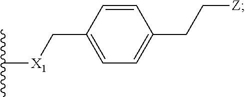

R⁵ is selected from hydrogen and C₁₋₆ alkyl;

Z is selected from:

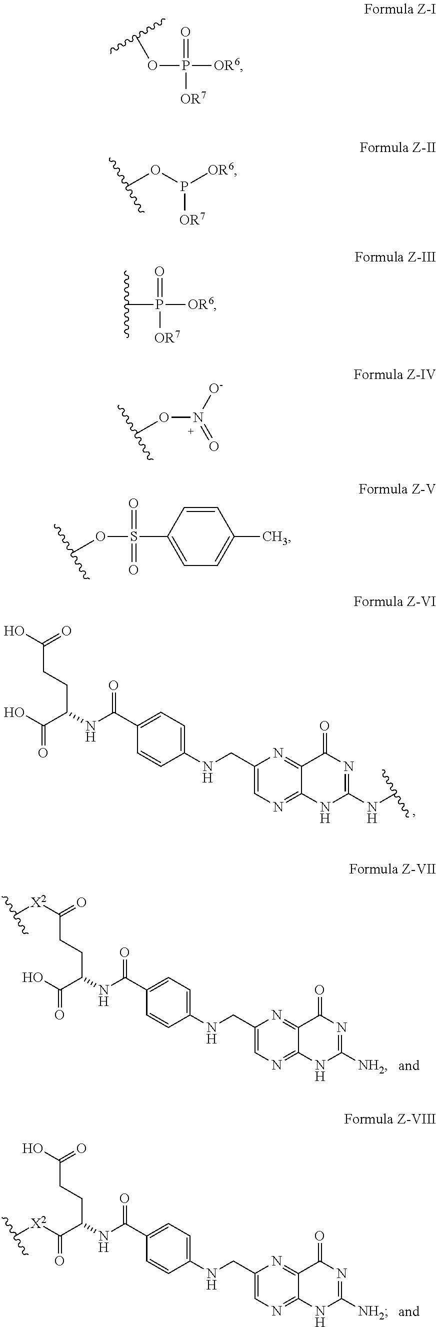

R⁶ and R⁷ are independently selected from hydrogen, C₁₋₆-alkyl, and polyethylene glycol residue.

In some embodiments, X¹ is —NR⁵—, and R⁵ is selected from hydrogen, methyl, and ethyl. In other embodiments, X¹ is —O—.

In certain embodiments, Z is

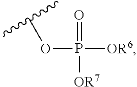

R⁶ is selected from ethyl and a polyethylene glycol residue, and R⁷ is selected from hydrogen and ethyl.

In still other embodiments, A is selected from:

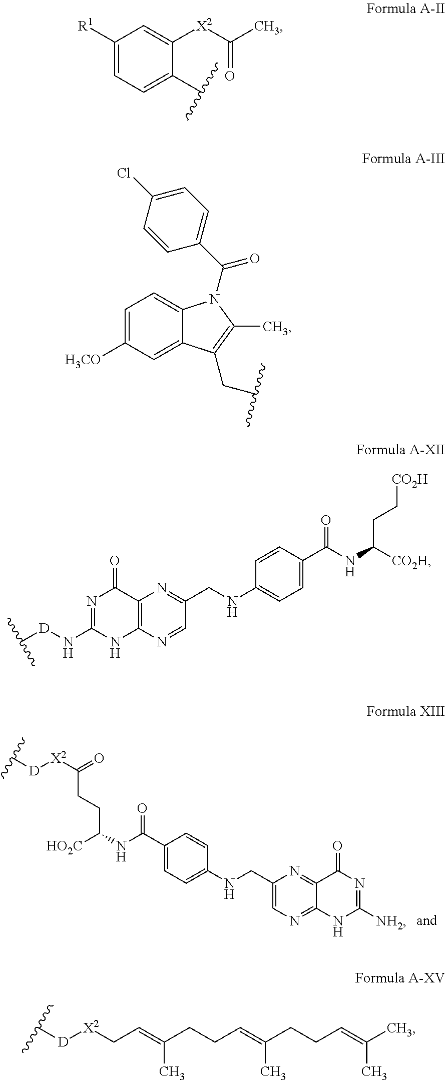

wherein

D is

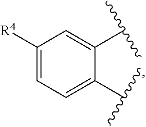

R¹ and R⁴ are independently selected from hydrogen and trifluoromethyl, and X² is selected from —O—, —S—, and —NH—.

In some embodiments, X¹ is —O—, Z is —O—P(O)(CH₂CH₃)₂, and A is:

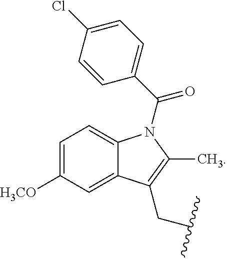 [SMILES: *Cc1c(C)n(C(=O)c2ccc(Cl)cc2)c2ccc(OC)cc12]

In certain embodiments, X¹ is selected from —O— and —NH—, Z is —O—P(O)(CH₂CH₃)₂, A is:

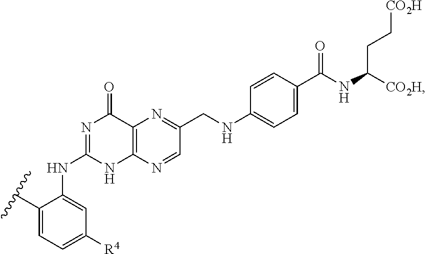

and R⁴ is selected from hydrogen and trifluoromethyl.

In other embodiments, X¹ and X² are independently selected from —O— and —NH—, Z is —O—P(O)(CH₂CH₃)₂, A is:

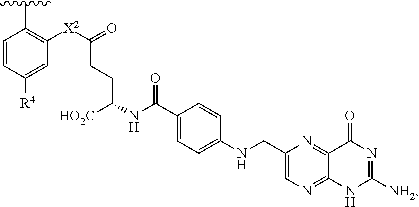

and R⁴ is selected from hydrogen and trifluoromethyl.

In some embodiments, X¹ and X² are independently selected from —O—, —S—, and —NH—; Z is —O—P(O)(CH₂CH₃)₂; and A is:

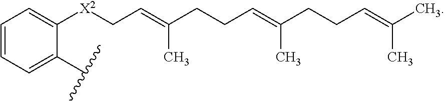

In some embodiments, X¹ is selected from —O—, —S—, and —NH—, Z is selected from —O—P(O)(CH₂CH₃)₂ and —ONO₂, A is:

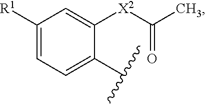

and R¹ is selected from hydrogen and trifluoromethyl, and X² is selected from —O—, —S— and —NH—.

In certain embodiments, X¹ is selected from —O— and —NH—, Z is —ONO₂, and A is:

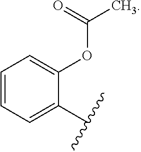 [SMILES: *c1ccccc1OC(C)=O]

Accordingly, the compounds of Formula I include but are not limited to compounds of which the structures are shown below:

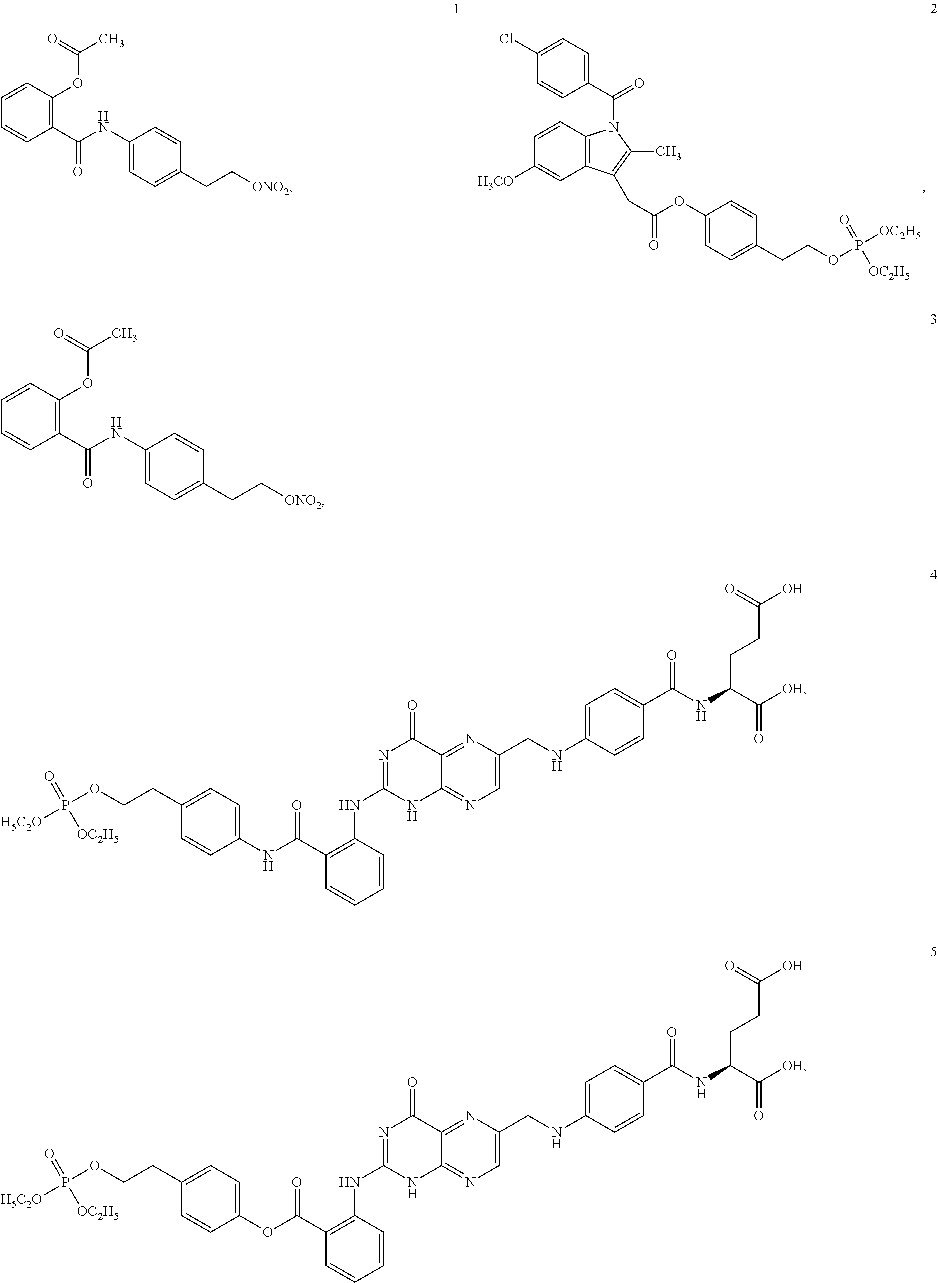

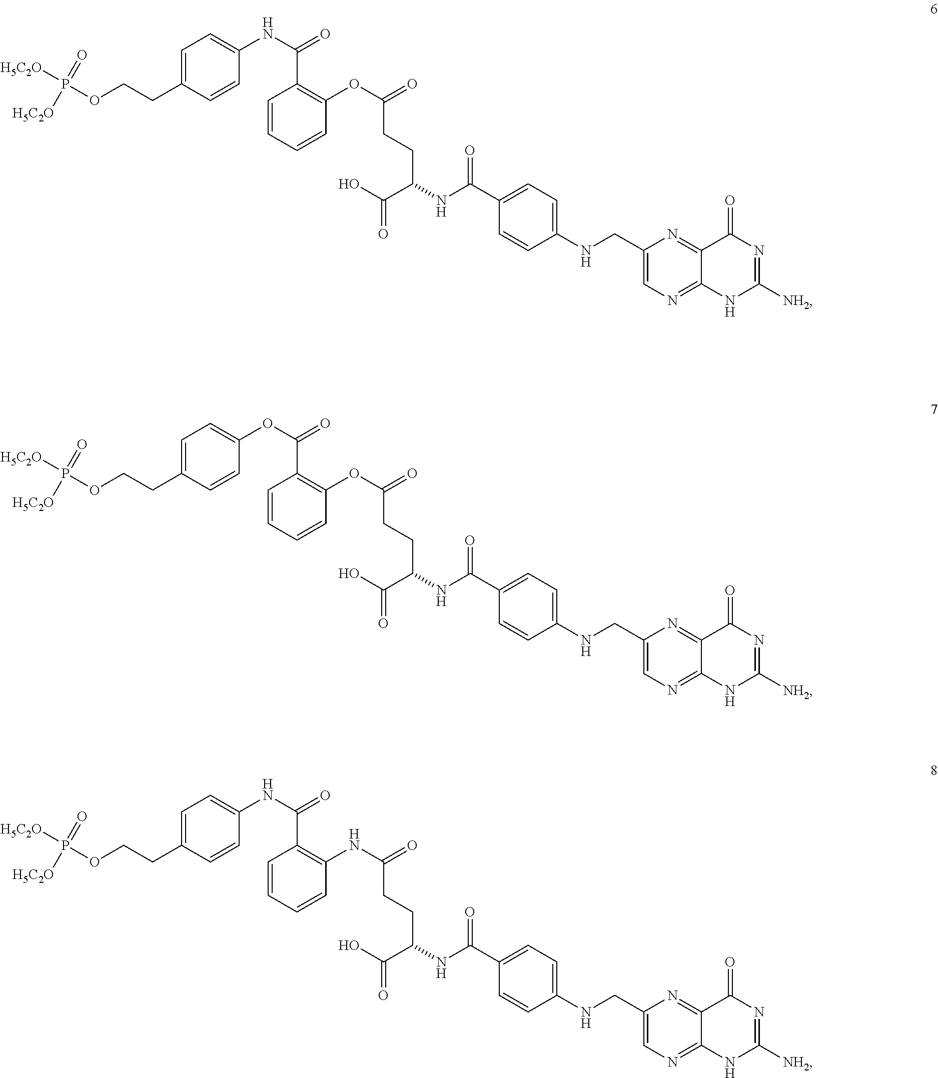

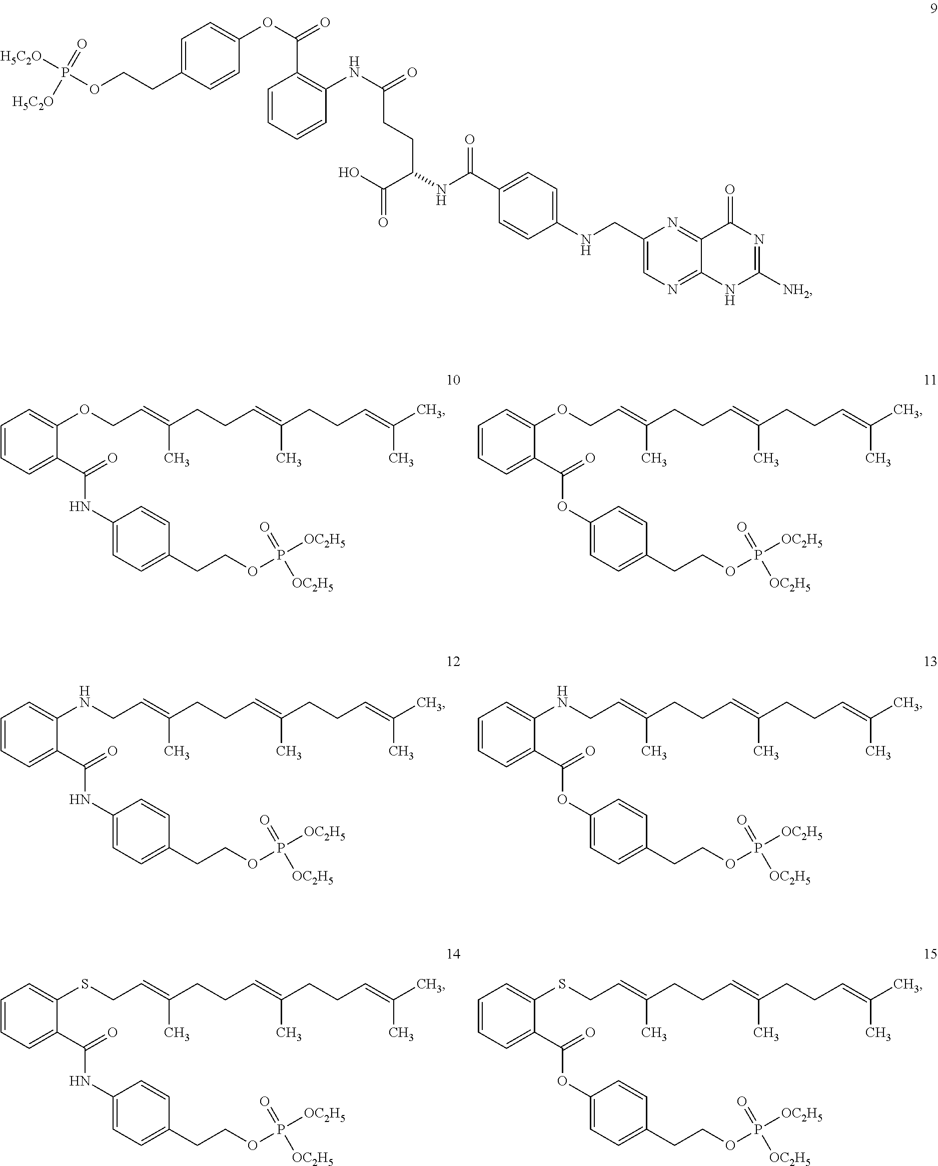

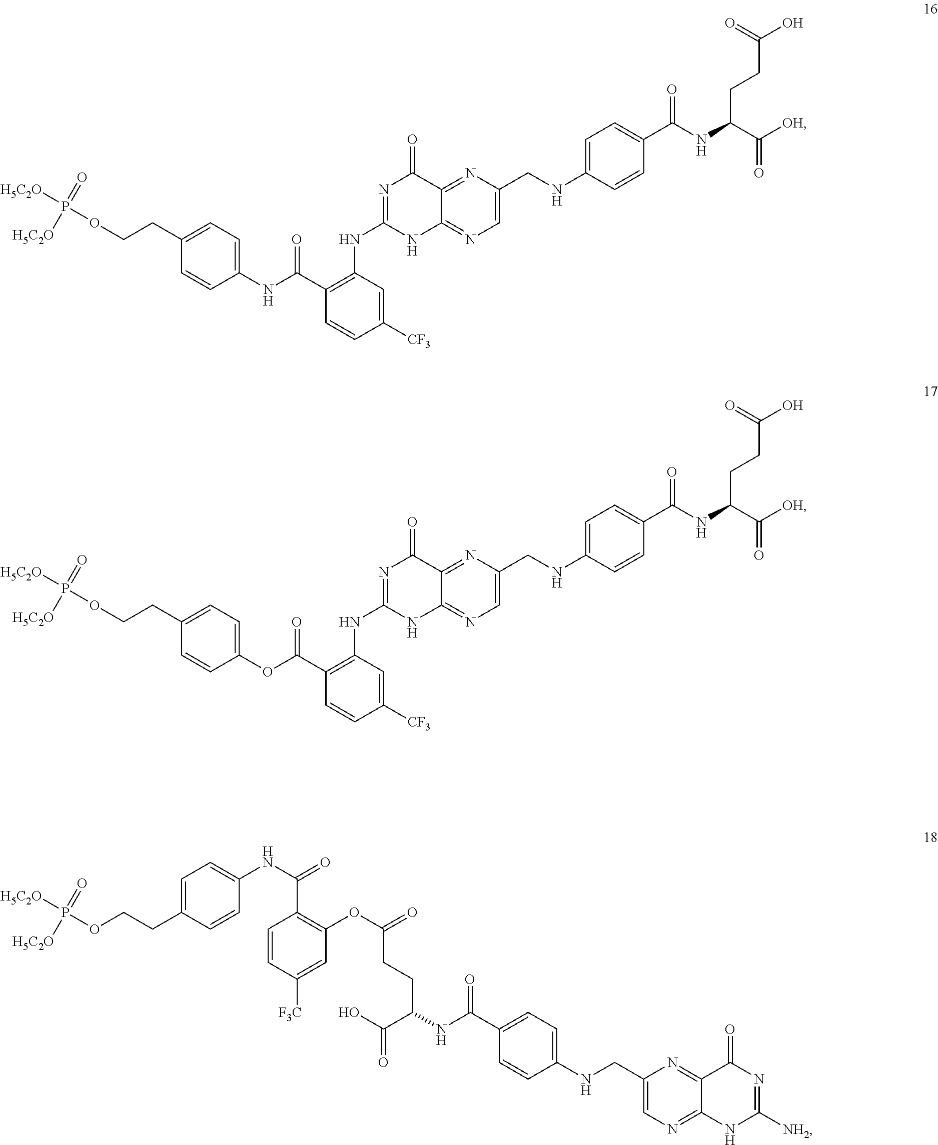

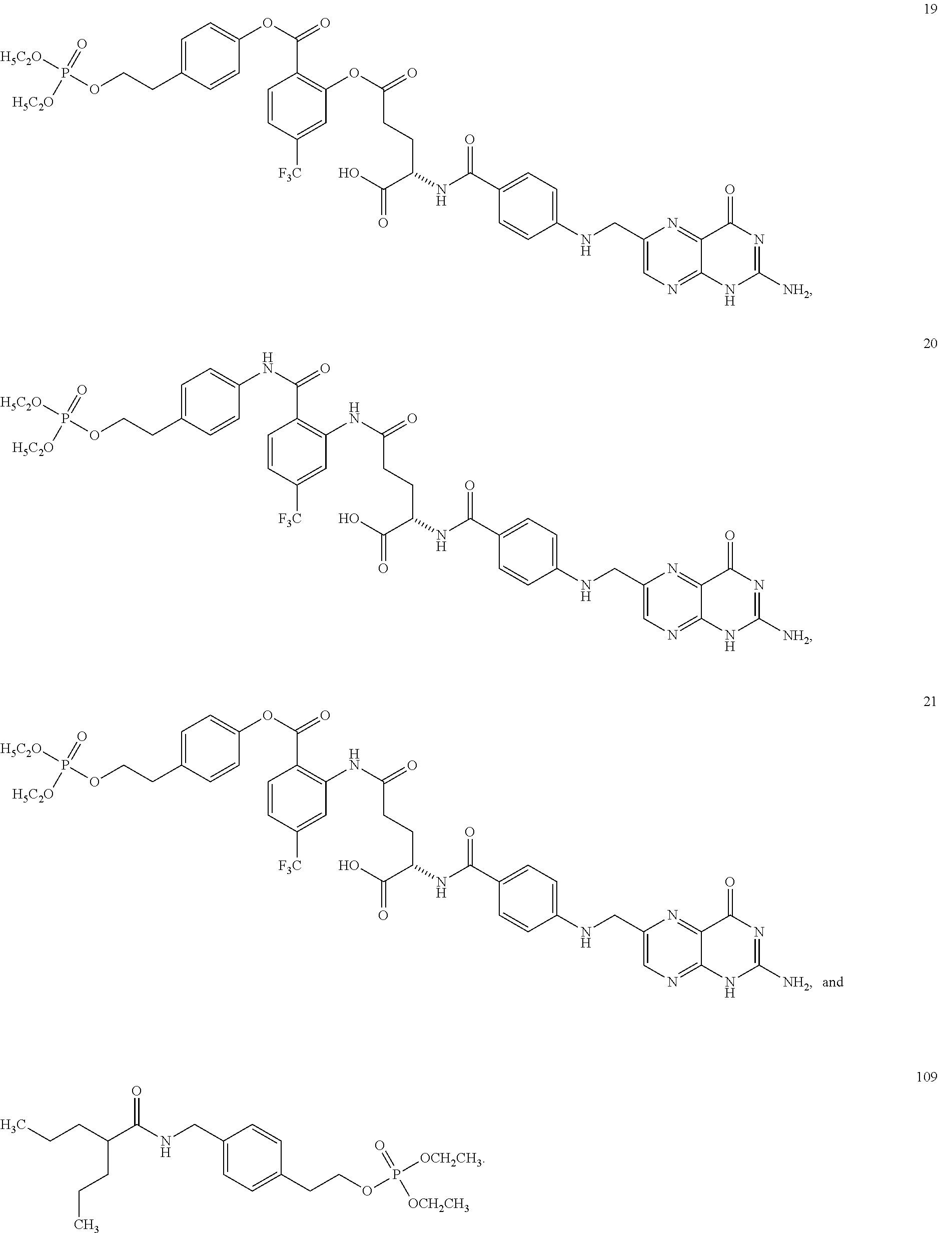

In a second aspect the invention features a compound of general Formula II

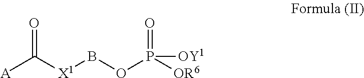

or a pharmaceutically acceptable salt thereof.

In Formula II: Y¹ is a polyethylene glycol residue; R⁶ is selected from hydrogen, C₁₋₆-alkyl, and polyethylene glycol residue;

A is an optionally substituted aliphatic, heteroaliphatic, aromatic, heteroaromatic substituent or alkylaryl substituent having 1 to 100 carbon atoms or selected from:

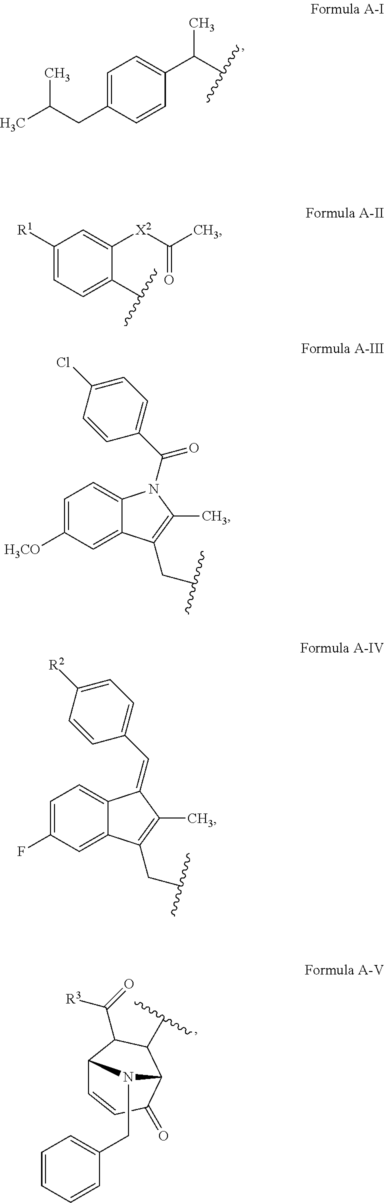

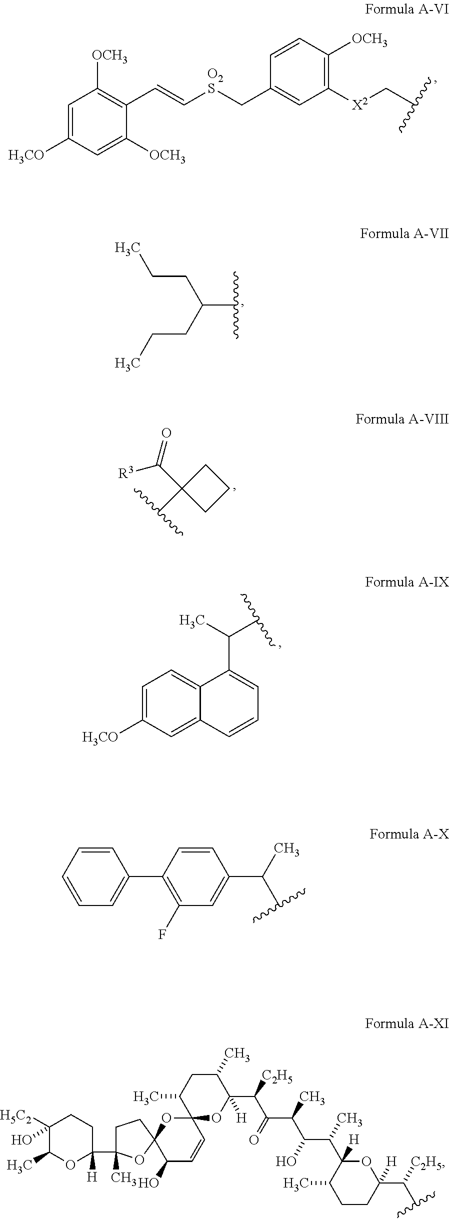

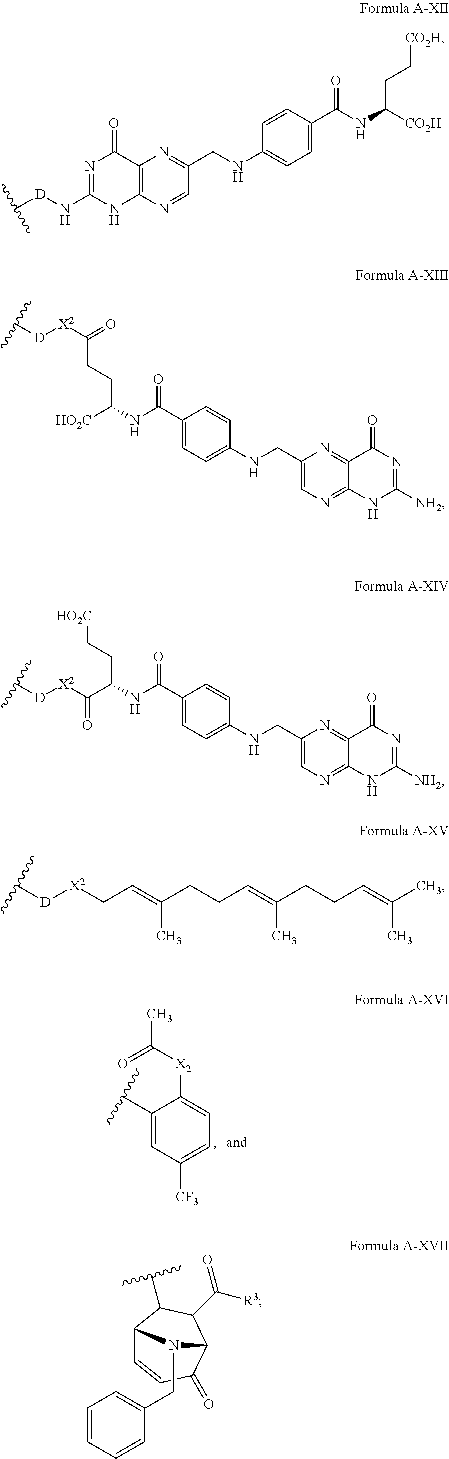

D is absent or

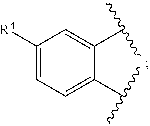

X¹ and X² are independently selected from —O—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B—Z;

R⁵ is selected from hydrogen and C₁₋₆ alkyl;

B is selected from:

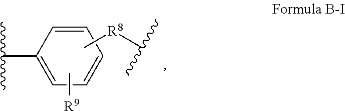

a single bond, and an aliphatic group with 1 to 22 carbon atoms;

R⁸ is a C₁₋₄ alkylene; and

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)C—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl.

In further embodiments, Y¹ is a polyethylene glycol residue described by —O(CH₂CH₂O)ₘR¹⁰, wherein m is 1 to 100 (e.g. 20 to 100, 20 to 50, 40 to 50), and R¹⁰ is selected from hydrogen, alkyl and alkoxy, and R⁶ is hydrogen.

In still other embodiments, Y¹ is —O(CH₂CH₂O)ₘR¹⁰ wherein m is 45, R¹⁰ is —OCH₃, and R⁶ is hydrogen.

In some embodiments, X¹ is —O—.

In other embodiments, X¹ is —NR⁵— and R⁵ is selected from hydrogen, methyl, and ethyl.

In certain embodiments, B is —(CH₂)₄—.

In some embodiments, A is:

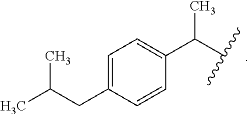 [SMILES: *C(C)c1ccc(CC(C)C)cc1]

In other embodiments, the compound is:

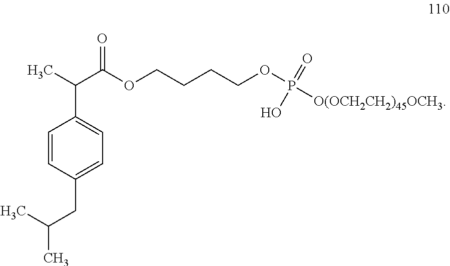

In a third aspect, the invention features a compound of general Formula III

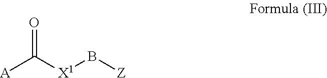

or a pharmaceutically acceptable salt thereof.

In Formula III: A is selected from:

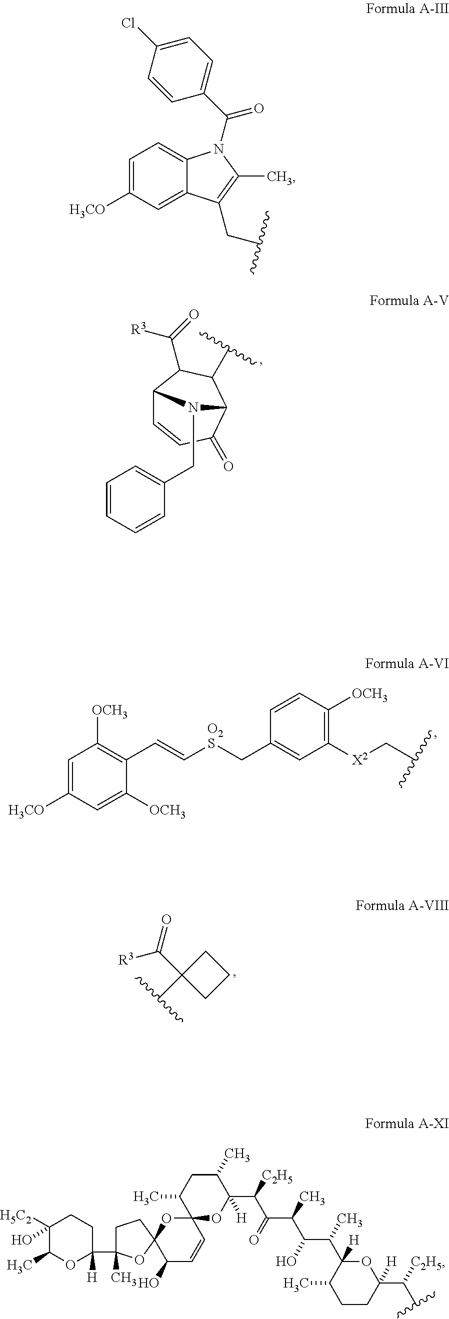

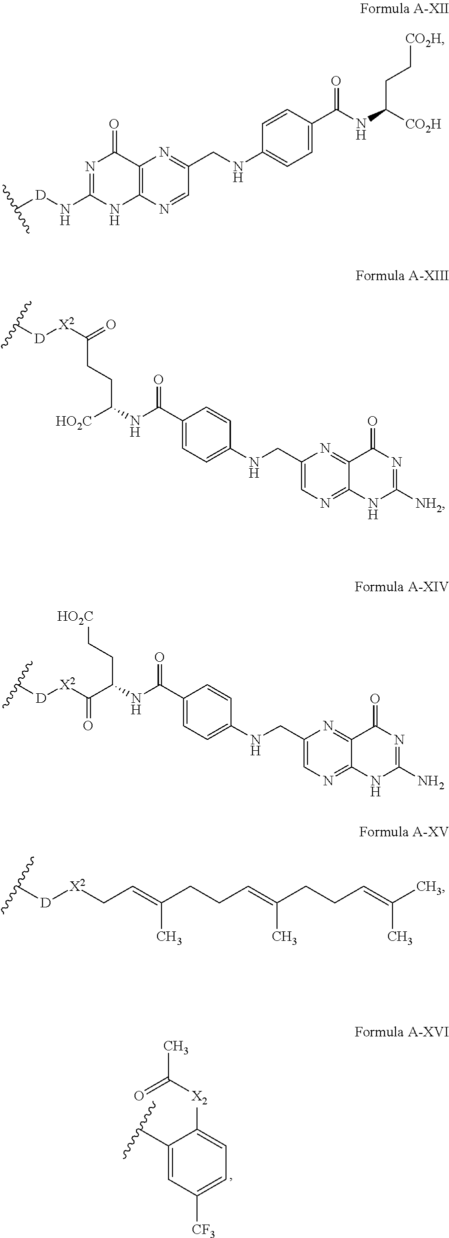

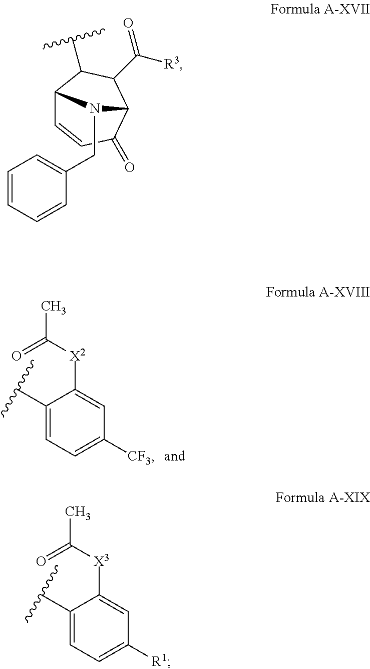

D is absent or

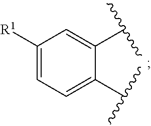

X¹ and X² are independently selected from —O—, —NR⁵—, and —S—;

R¹ and R⁴ are independently selected from hydrogen and trifluoromethyl;

X³ is selected from —S— and —NH—;

R³ is selected from hydroxyl, Z, and —X¹—B—Z;

R⁵ is selected from hydrogen and C₁₋₆ alkyl;

B is selected from:

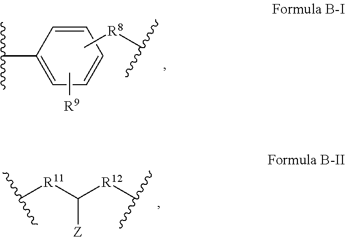

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;

Z is selected from:

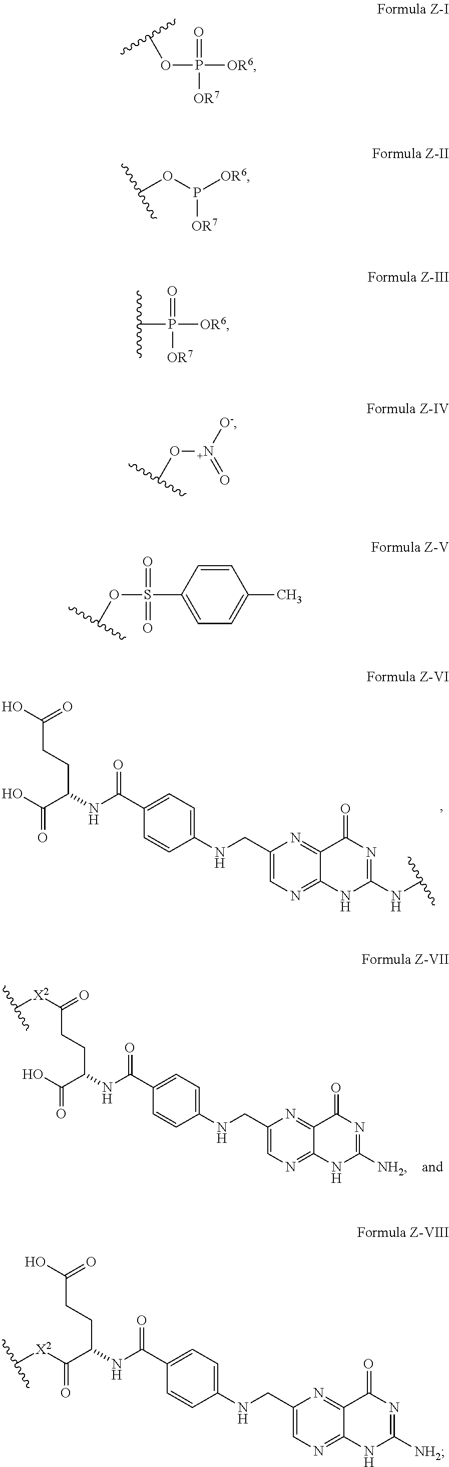

or B together with Z forms a structure:

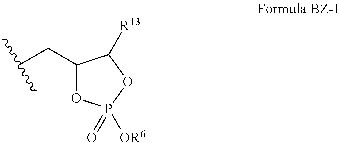

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

In still other embodiments, X¹ is —O—.

In certain embodiments, X¹ is —NR⁵— and R⁵ is selected from hydrogen, methyl, and ethyl.

In some embodiments, B is selected from:

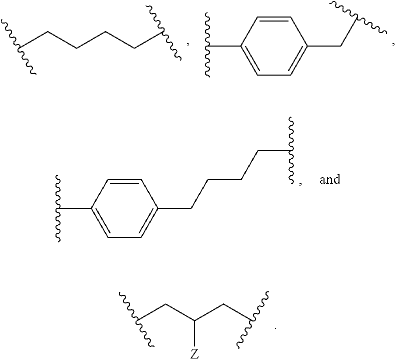

In other embodiments, Z is selected from —OP(O)(OCH₂CH₃)₂ and —ONO₂.

In further embodiments, BZ is

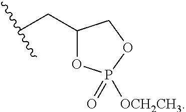 [SMILES: *CC1COP(=O)(OCC)O1]

In certain embodiments, X¹ is selected from —O— and —NH—, B is selected from

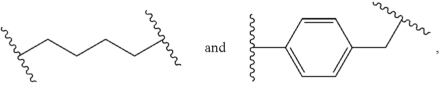 [SMILES: *CCCC* ; *Cc1ccc(*)cc1]

### Z is —OP(O)(OCH₂CH₃)₂, and A is:

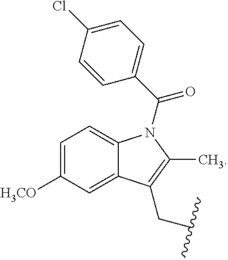 [SMILES: *Cc1c(C)n(C(=O)c2ccc(Cl)cc2)c2ccc(OC)cc12]

In some embodiments, X¹ is selected from —O— and —NH—, B is selected from

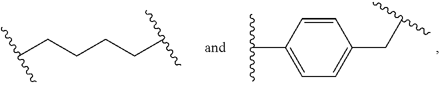 [SMILES: *CCCC* ; *Cc1ccc(*)cc1]

### Z is —OP(O)(OCH₂CH₃)₂, A is

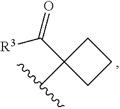

### and R³ is:

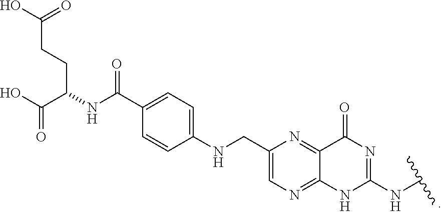

In some embodiments, wherein X¹ is selected from —O— and —NH—, B is selected from

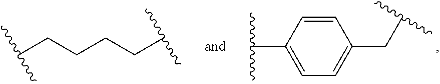 [SMILES: *CCCC* ; *Cc1ccc(*)cc1]

### Z is —OP(O)(OCH₂CH₃)₂, A is:

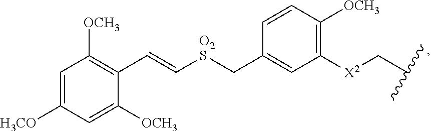

and X² is selected from —O— and —NH—.

In other embodiments, X¹ is selected from —O— and —NH—, B is selected from

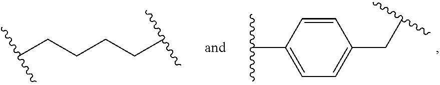 [SMILES: *CCCC* ; *Cc1ccc(*)cc1]

### Z is —OP(O)(OCH₂CH₃)₂, and A is:

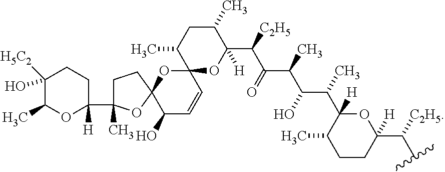

In further embodiments, X¹ is selected from —O— and —NH—, B is selected from

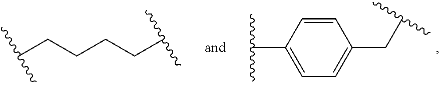 [SMILES: *CCCC* ; *Cc1ccc(*)cc1]

### Z is —OP(O)(OCH₂CH₃)₂, A is:

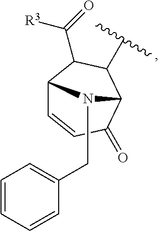

and R³ is hydroxyl or selected from:

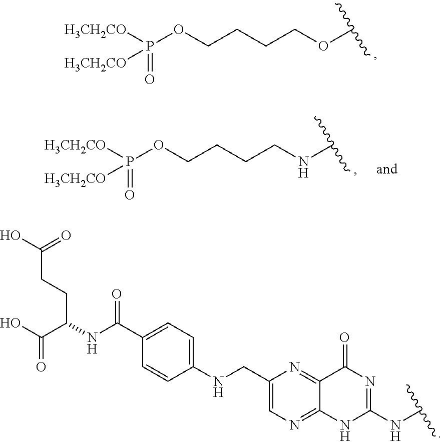

In certain embodiments, X¹ is selected from —O— and —NH—, B is selected from

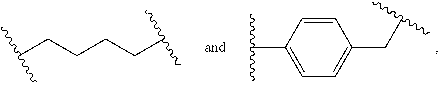 [SMILES: *CCCC* ; *Cc1ccc(*)cc1]

### Z is —OP(O)(OCH₂CH₃)₂, A is:

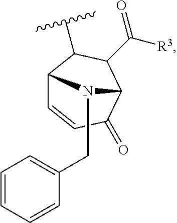

and R³ is hydroxyl or selected from:

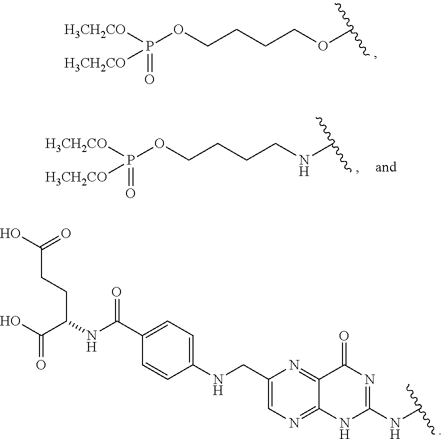

In some embodiments, X¹ is selected from —O— and —NH—, B is selected from

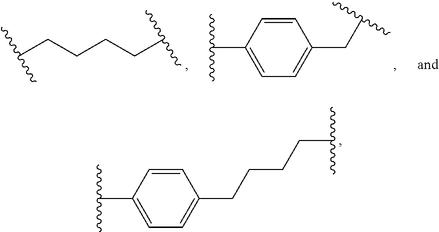 [SMILES: *CCCC* ; *Cc1ccc(*)cc1 ; *CCCCc1ccc(*)cc1]

### Z is —OP(O)(OCH₂CH₃)₂, A is:

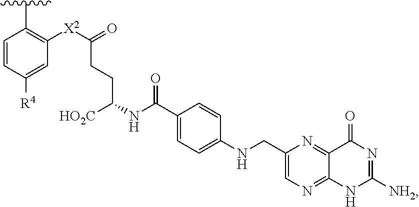

and R⁴ is selected from hydrogen and trifluoromethyl.

In some embodiments, X¹ is selected from —O— and —NH—, B is selected from

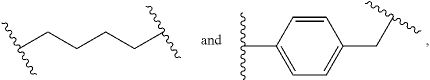 [SMILES: *CCCC* ; *Cc1ccc(*)cc1]

### Z is —OP(O)(OCH₂CH₃)₂, A is:

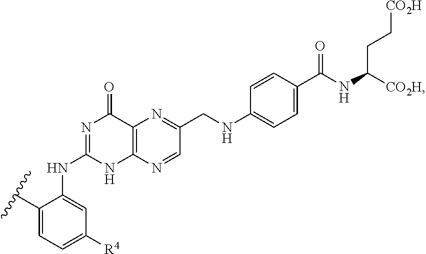

and R⁴ is selected from hydrogen and trifluoromethyl.

In other embodiments, X¹ is selected from —O— and —NH—, B is selected from

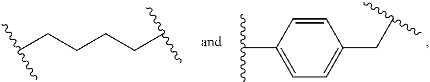 [SMILES: *CCCC* ; *Cc1ccc(*)cc1]

### Z is —OP(O)(OCH₂CH₃)₂, A is:

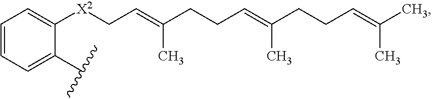

and X² is selected from —O—, —S—, and —NH—.

In other embodiments, X¹ is selected from —O— and —NH—, B is selected from

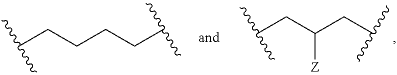

Z is selected from —OP(O)(OCH₂CH₃)₂ and —ONO₂, A is:

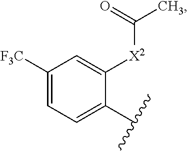

and X² is selected from —O—, —S—, and —NH—.

In some embodiments, X¹ is selected from —O— and —NH—, B is —(CH₂)₄—, Z is —ONO₂, A is:

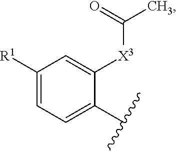

R¹ is selected from hydrogen and trifluoromethyl, and X³ is selected from —S—, and —NH—.

In other embodiments, X¹ is —NH—, A is:

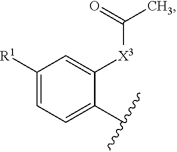

R¹ is selected from hydrogen and trifluoromethyl, and X³ is selected from —S—, and —NH—.

Accordingly, the compounds of Formula III include but are not limited to compounds of which the structures are shown below:

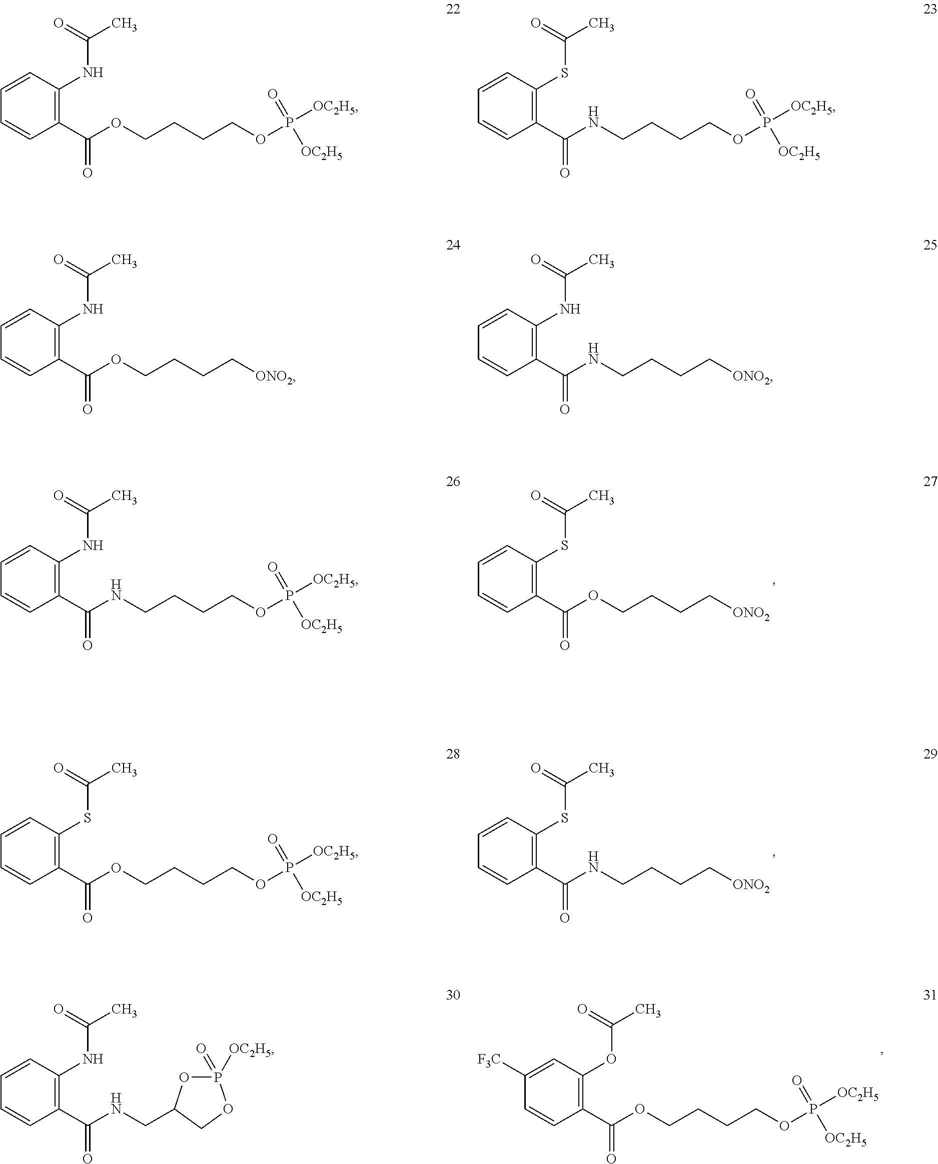

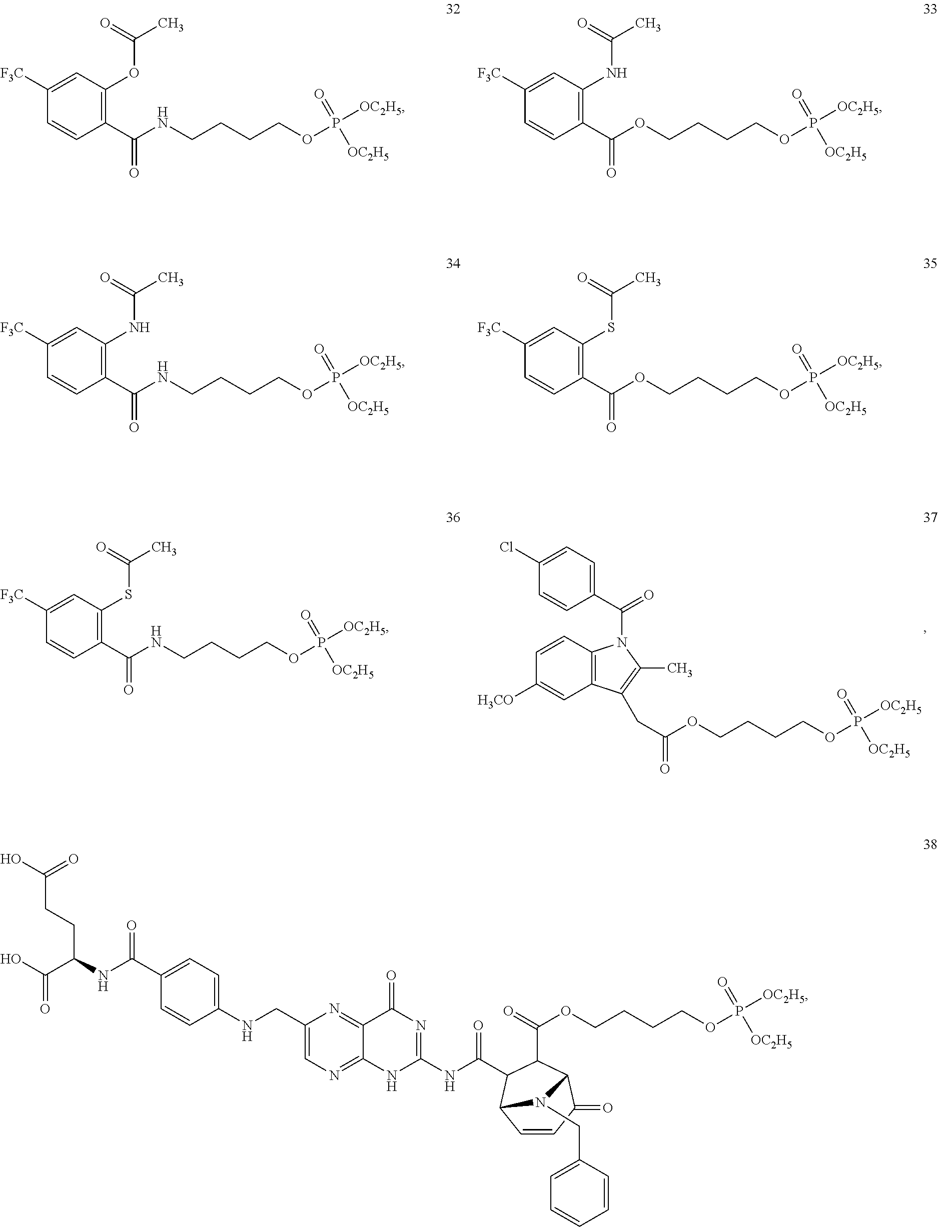

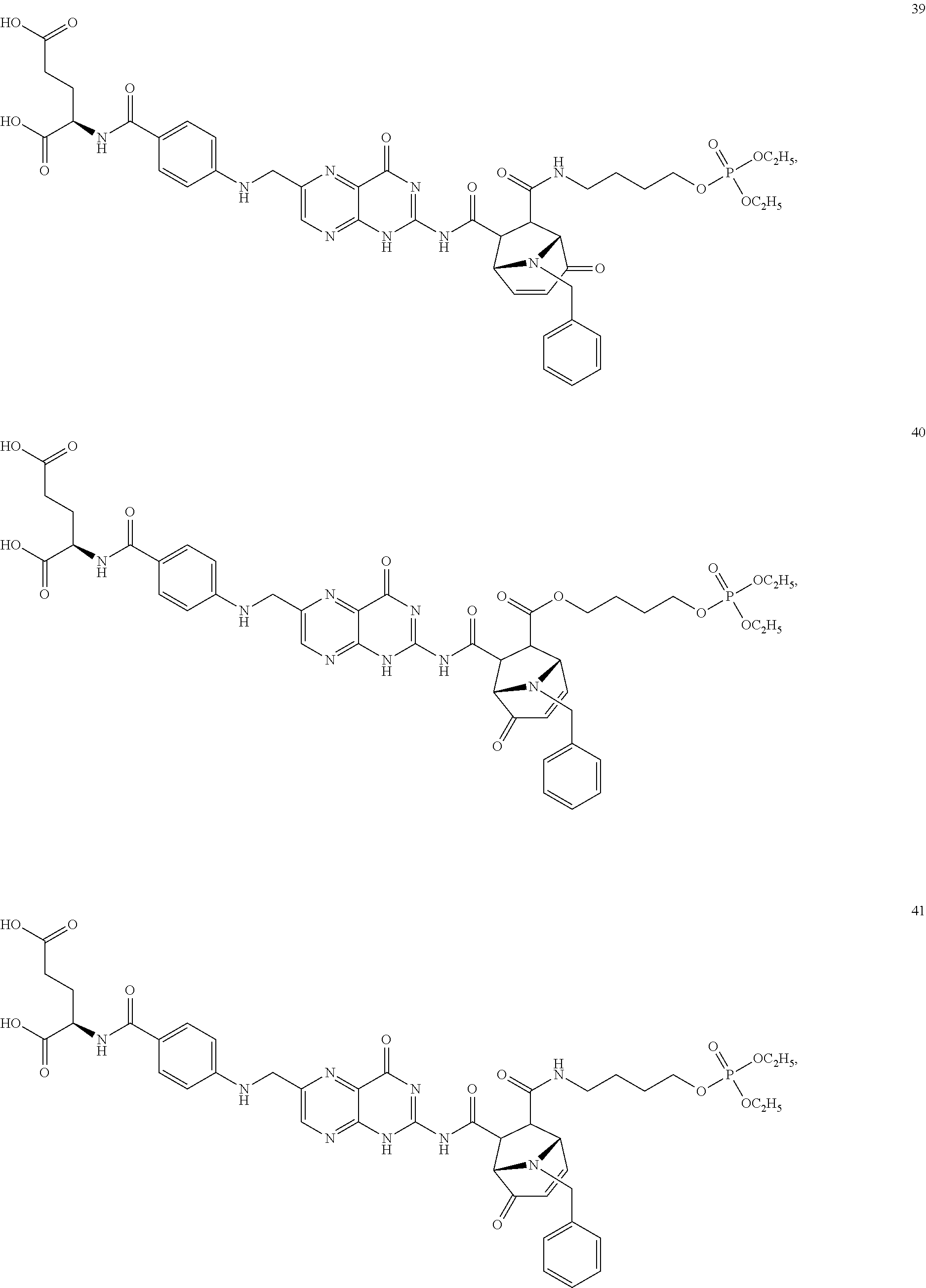 [SMILES: CCOP(=O)(OCC)OCCCCNC(=O)C1C(C(=O)Nc2nc(=O)c3nc(CNc4ccc(C(=O)N[C@H](CCC(=O)O)C(=O)O)cc4)cnc3[nH]2)[C@H]2C=CC(=O)[C@@H]1N2Cc1ccccc1 ; CCOP(=O)(OCC)OCCCCOC(=O)C1C(C(=O)Nc2nc(=O)c3nc(CNc4ccc(C(=O)N[C@H](CCC(=O)O)C(=O)O)cc4)cnc3[nH]2)[C@H]2C(=O)C=C[C@@H]1N2Cc1ccccc1 ; CCOP(=O)(OCC)OCCCCNC(=O)C1C(C(=O)Nc2nc(=O)c3nc(CNc4ccc(C(=O)N[C@H](CCC(=O)O)C(=O)O)cc4)cnc3[nH]2)[C@H]2C(=O)C=C[C@@H]1N2Cc1ccccc1]

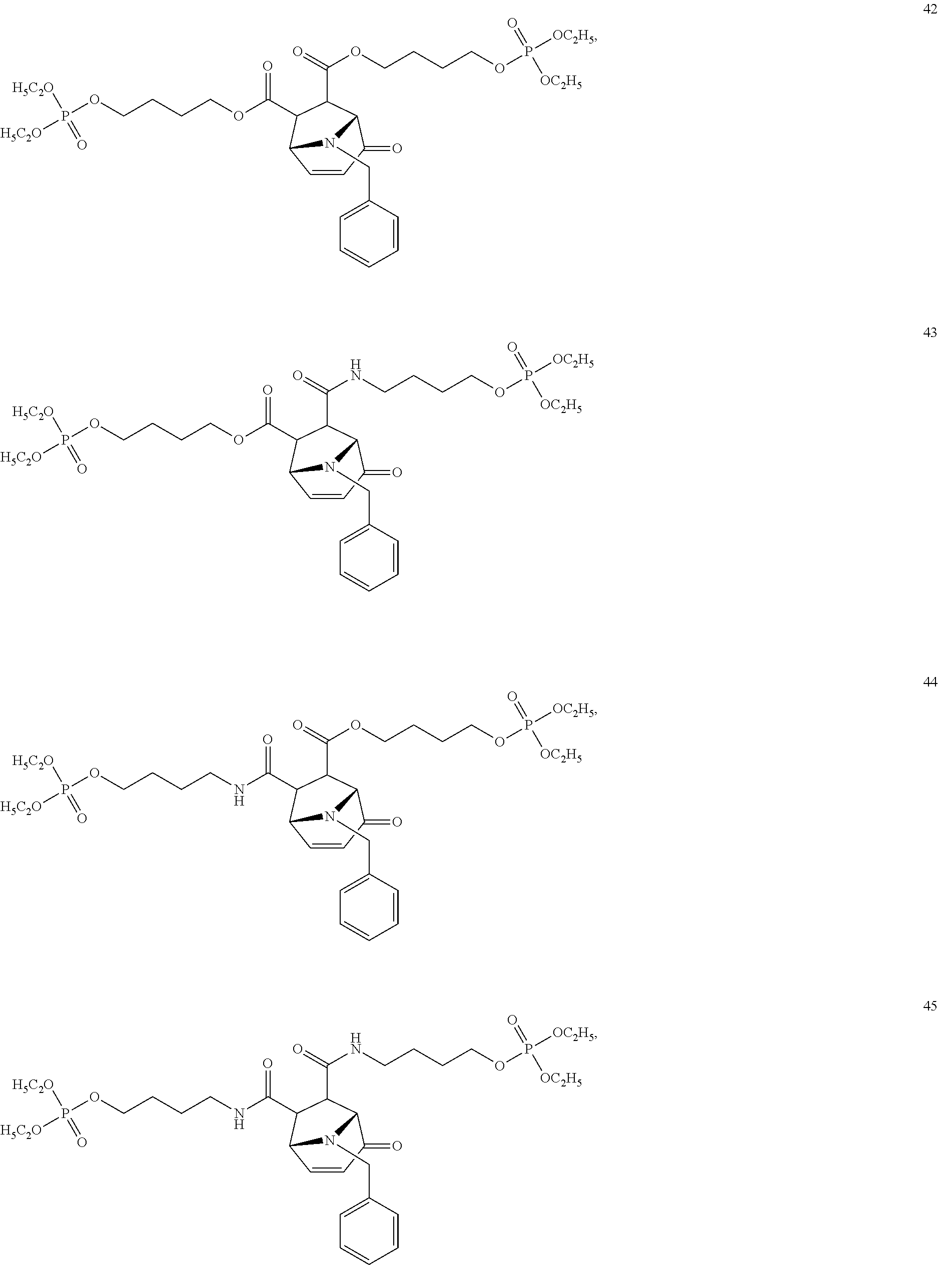

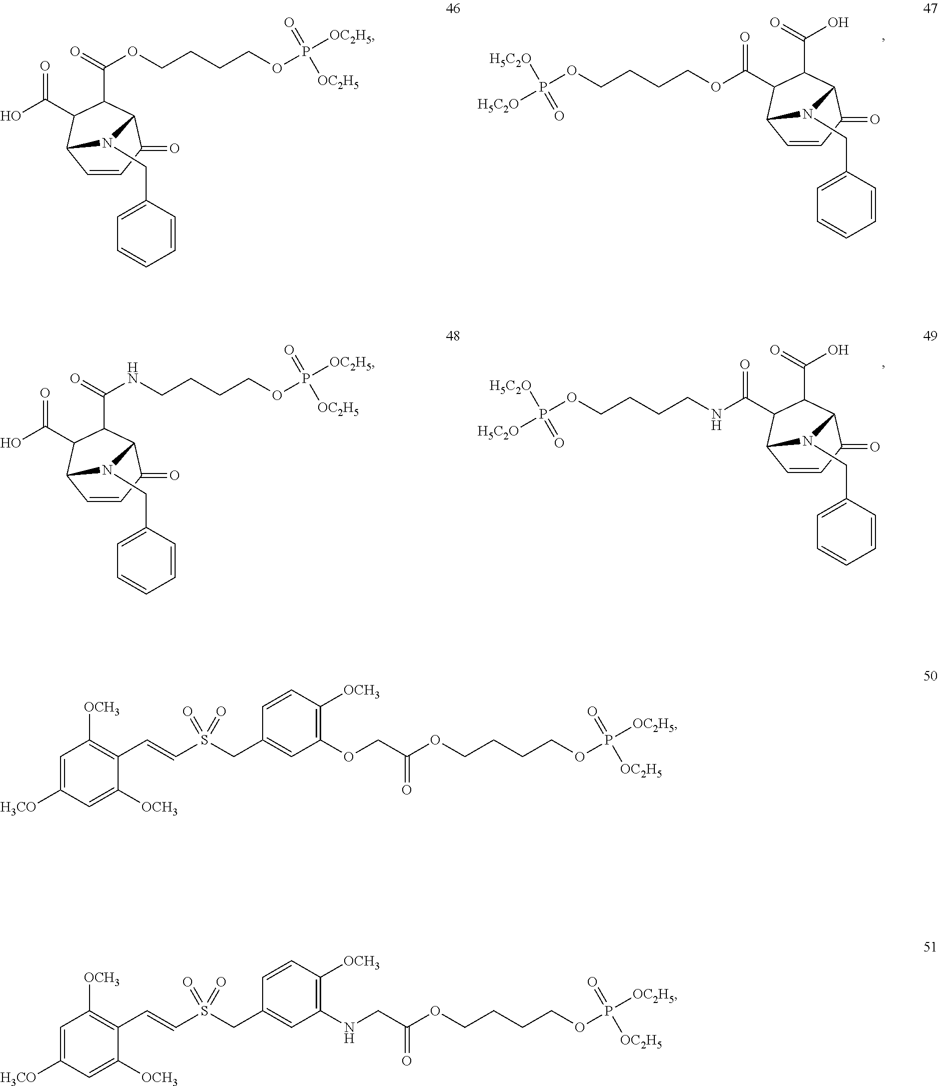

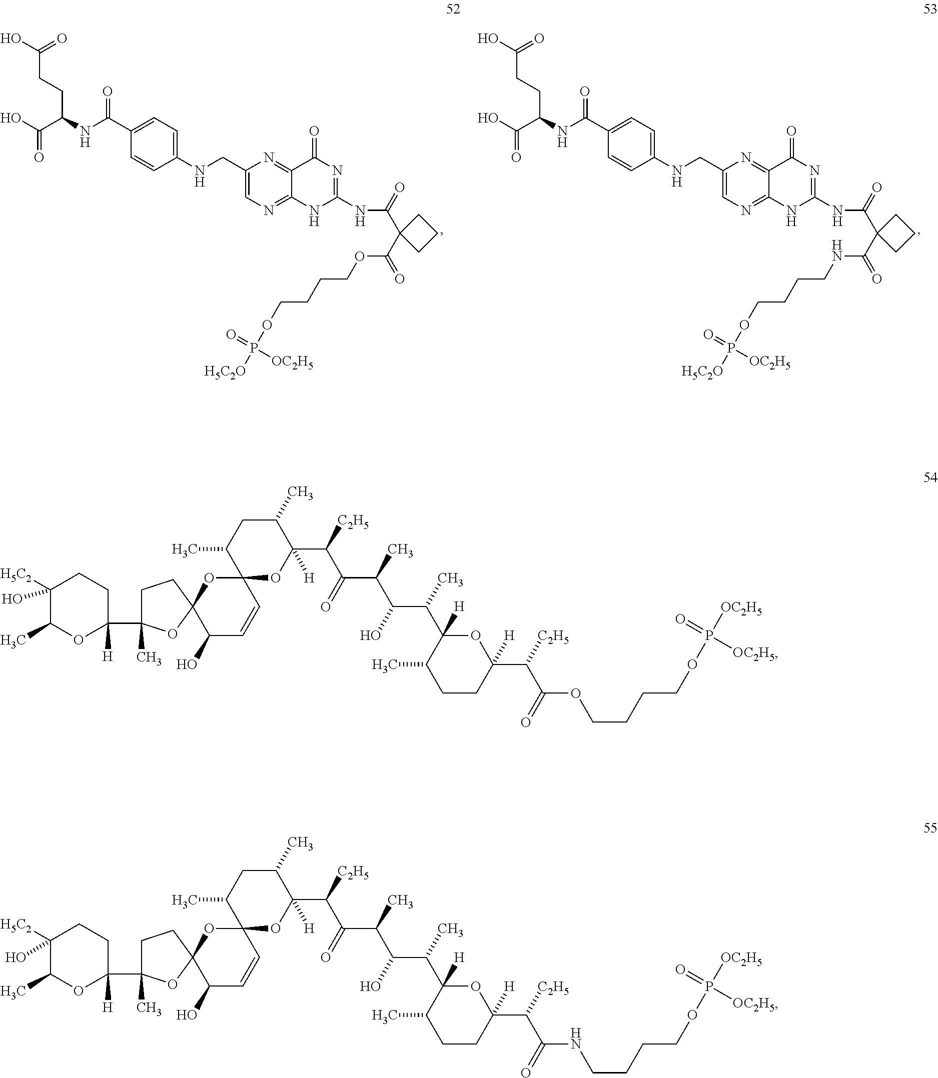

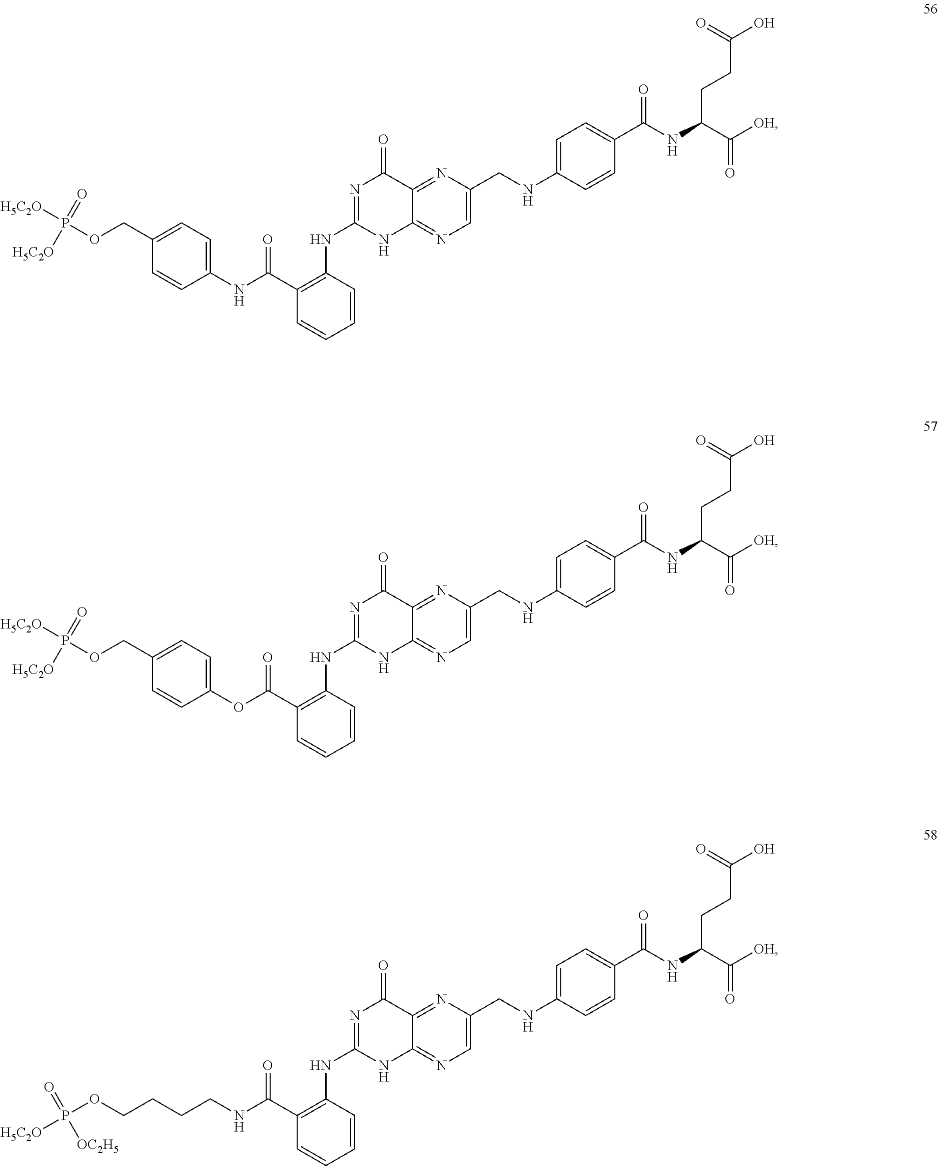

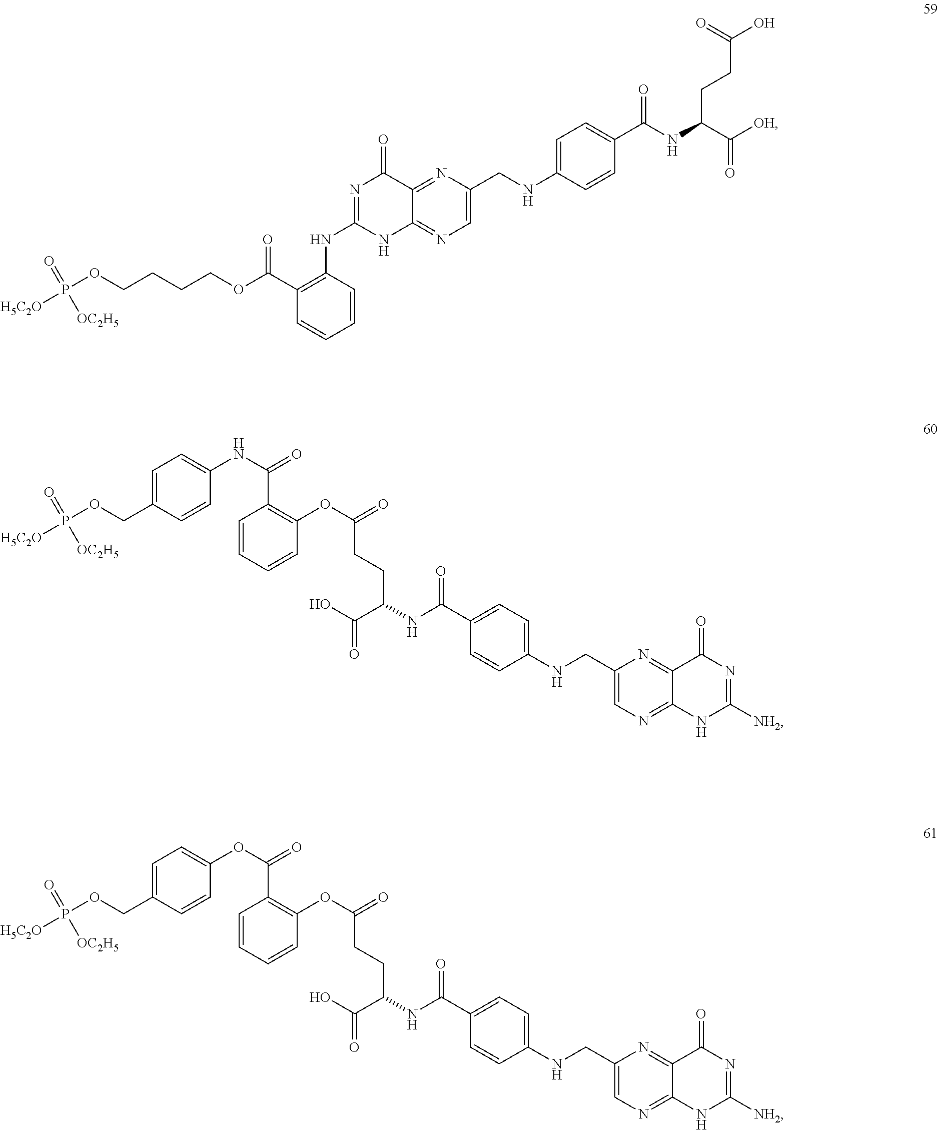

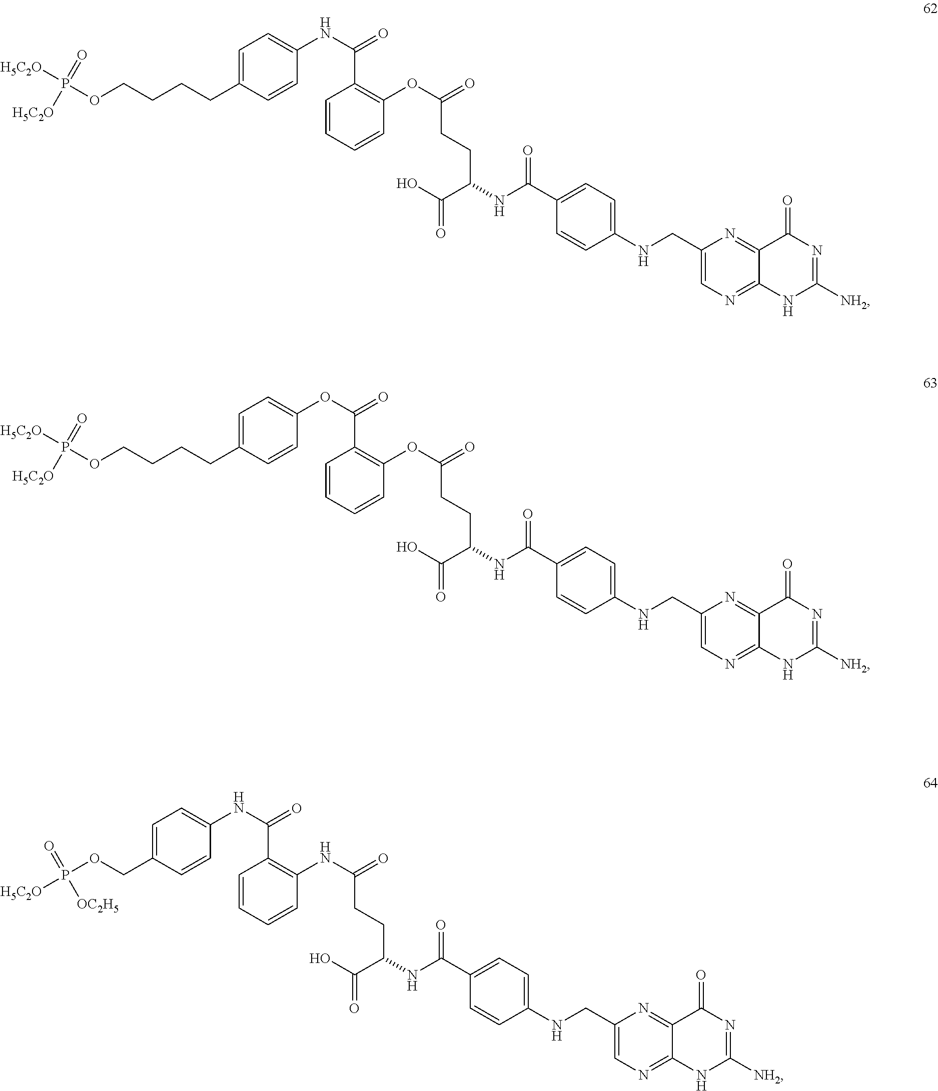

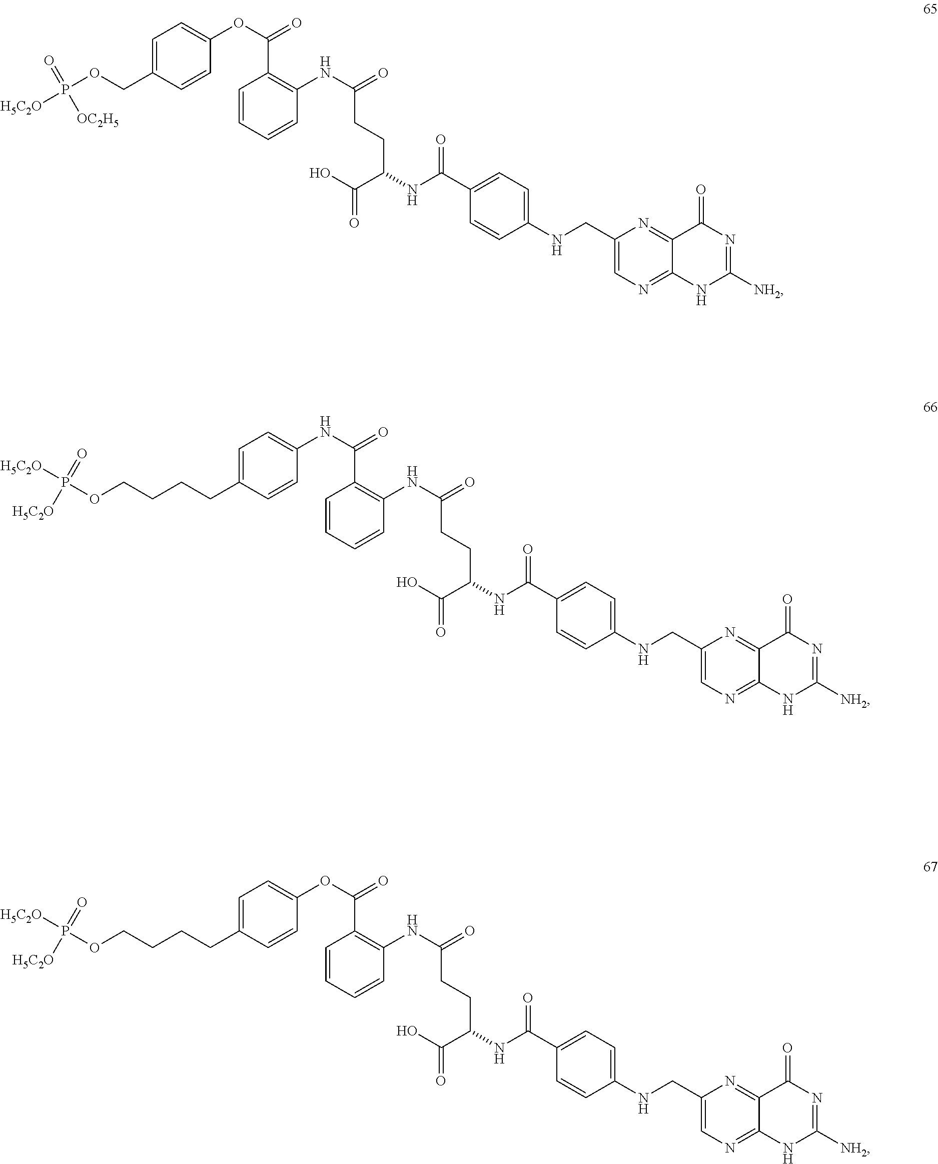

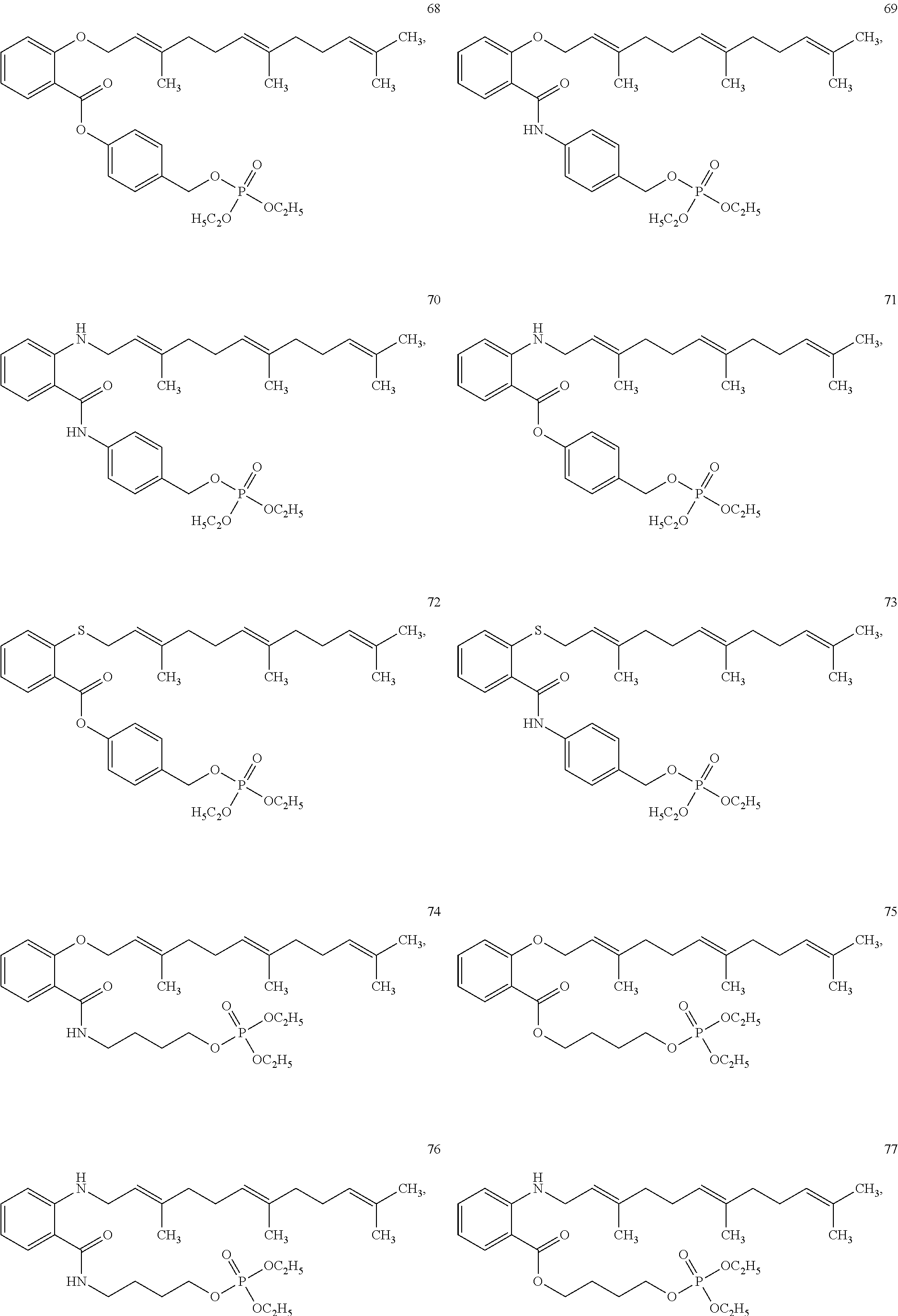

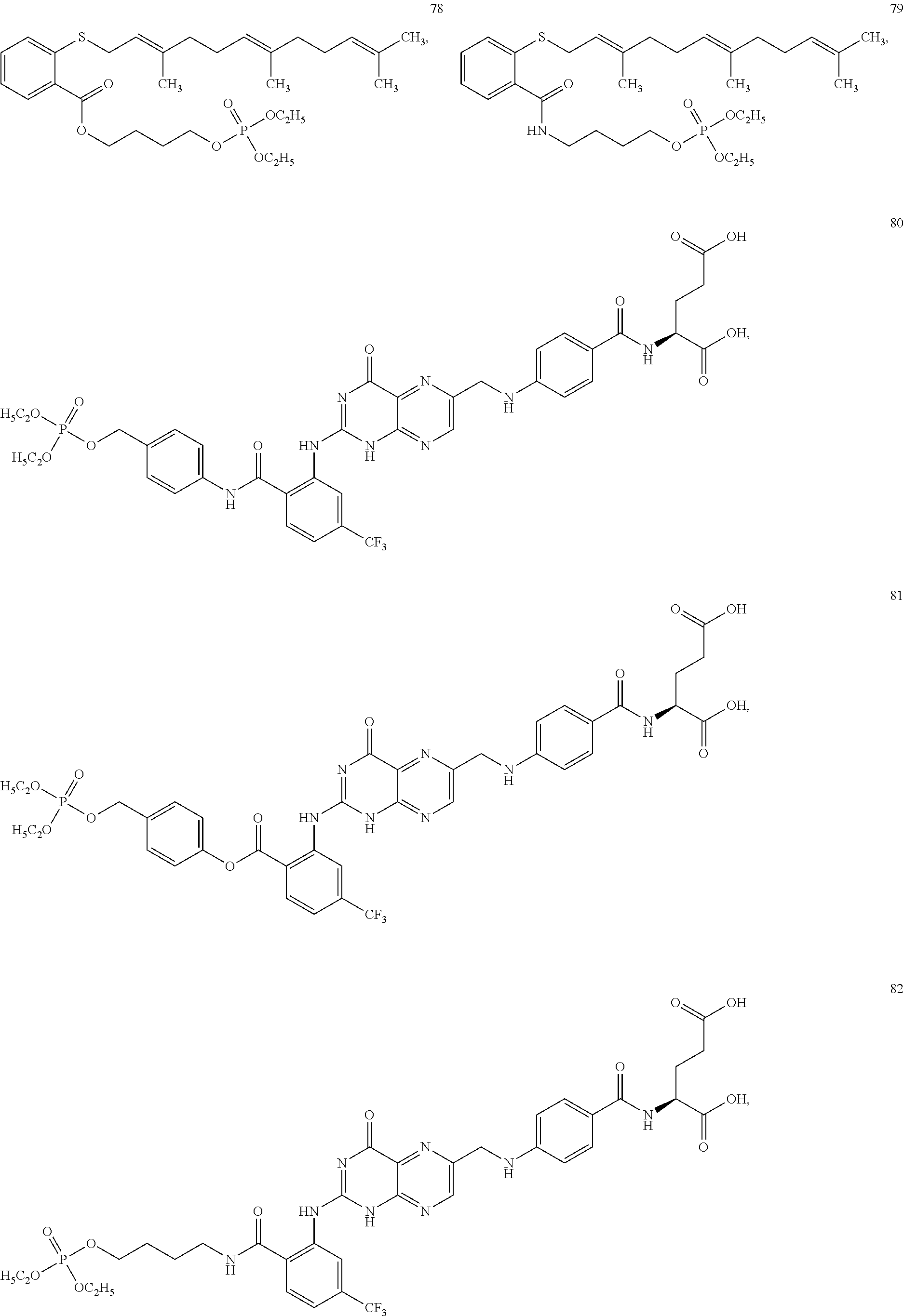

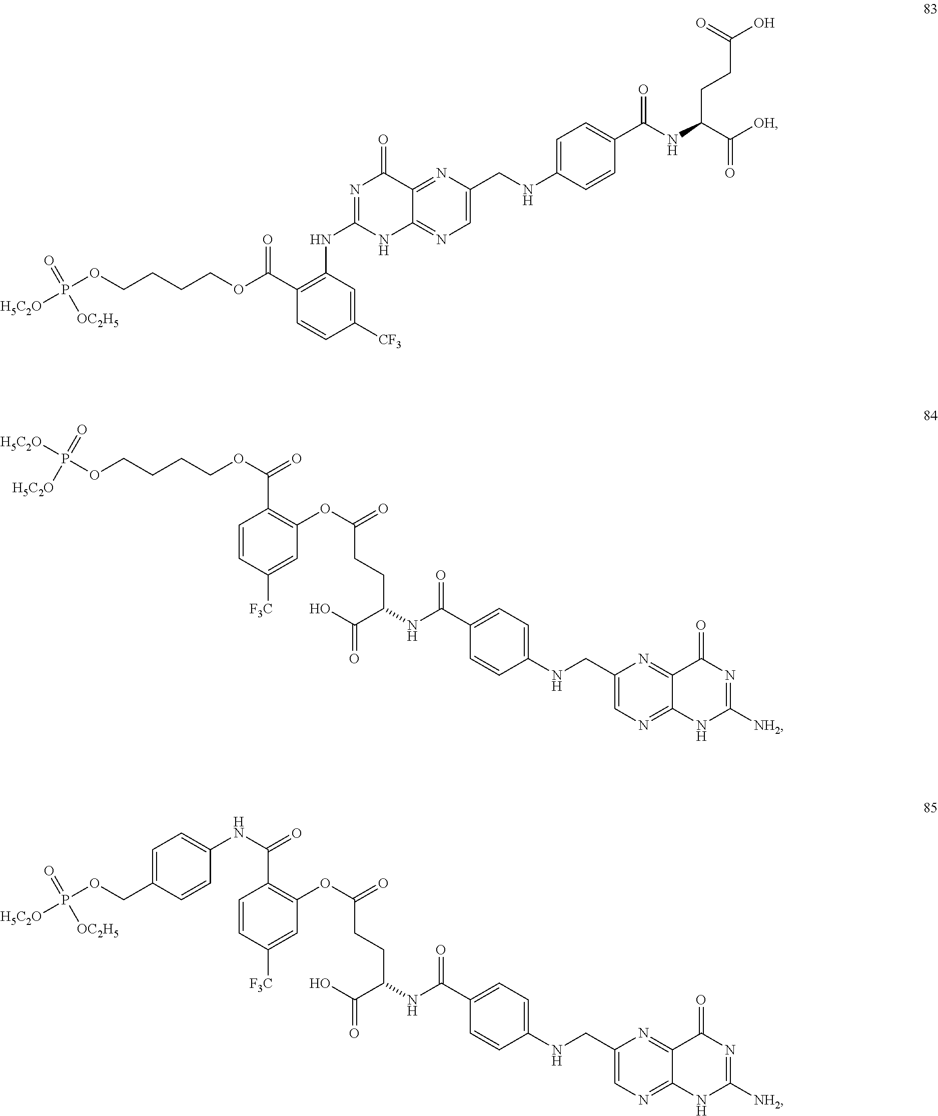

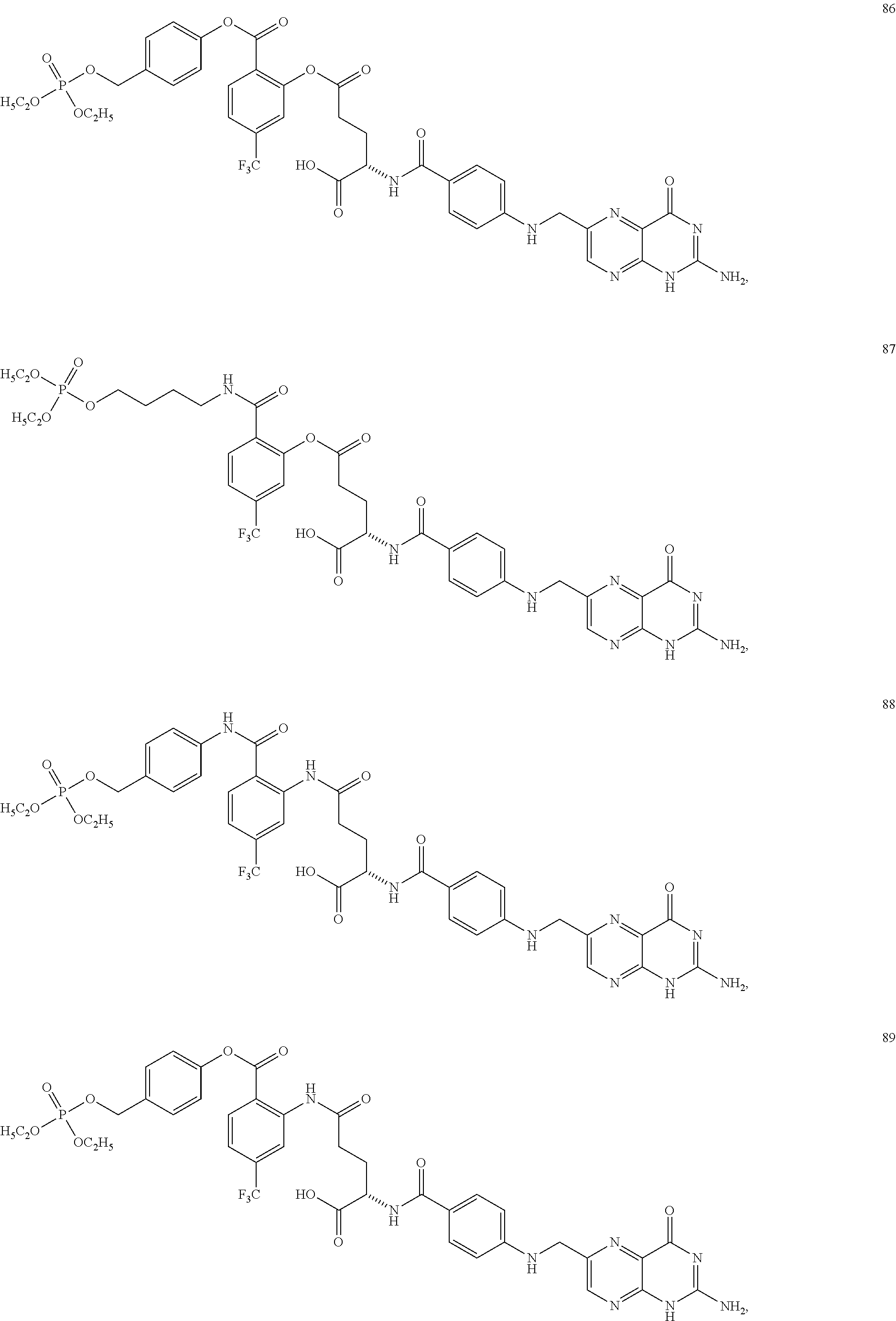

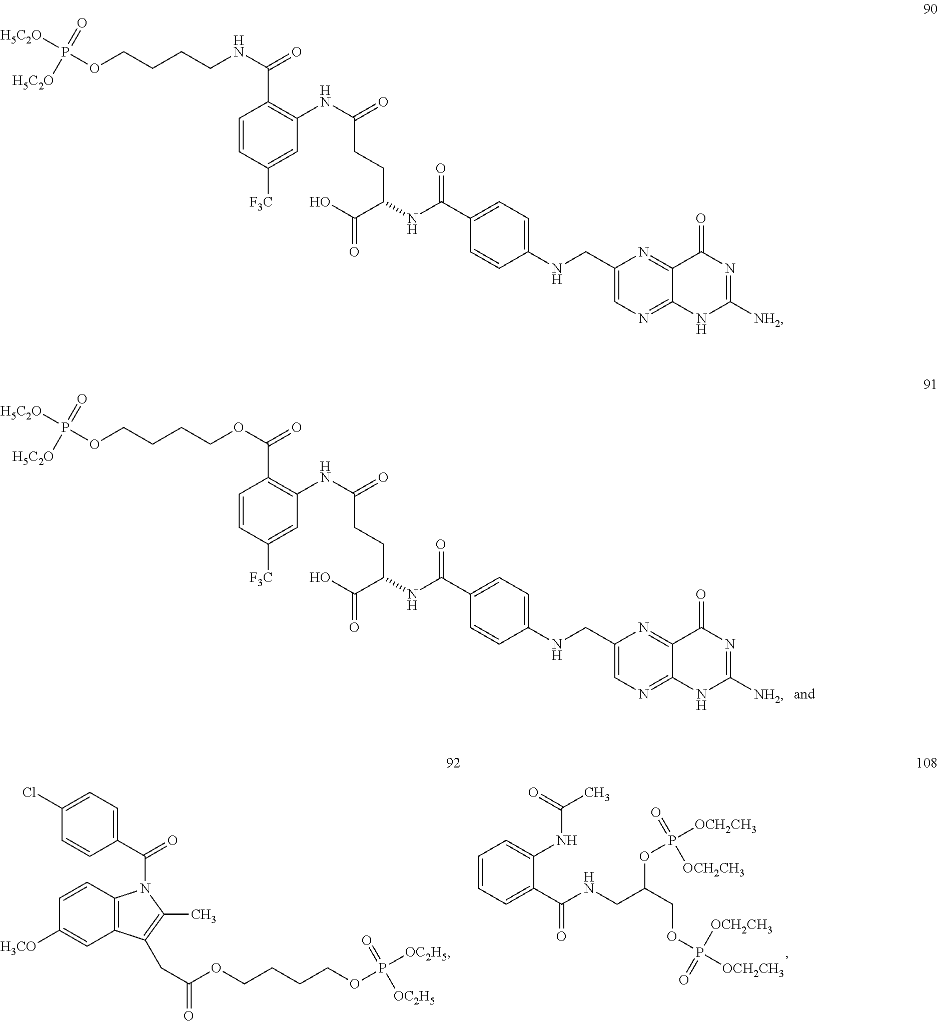

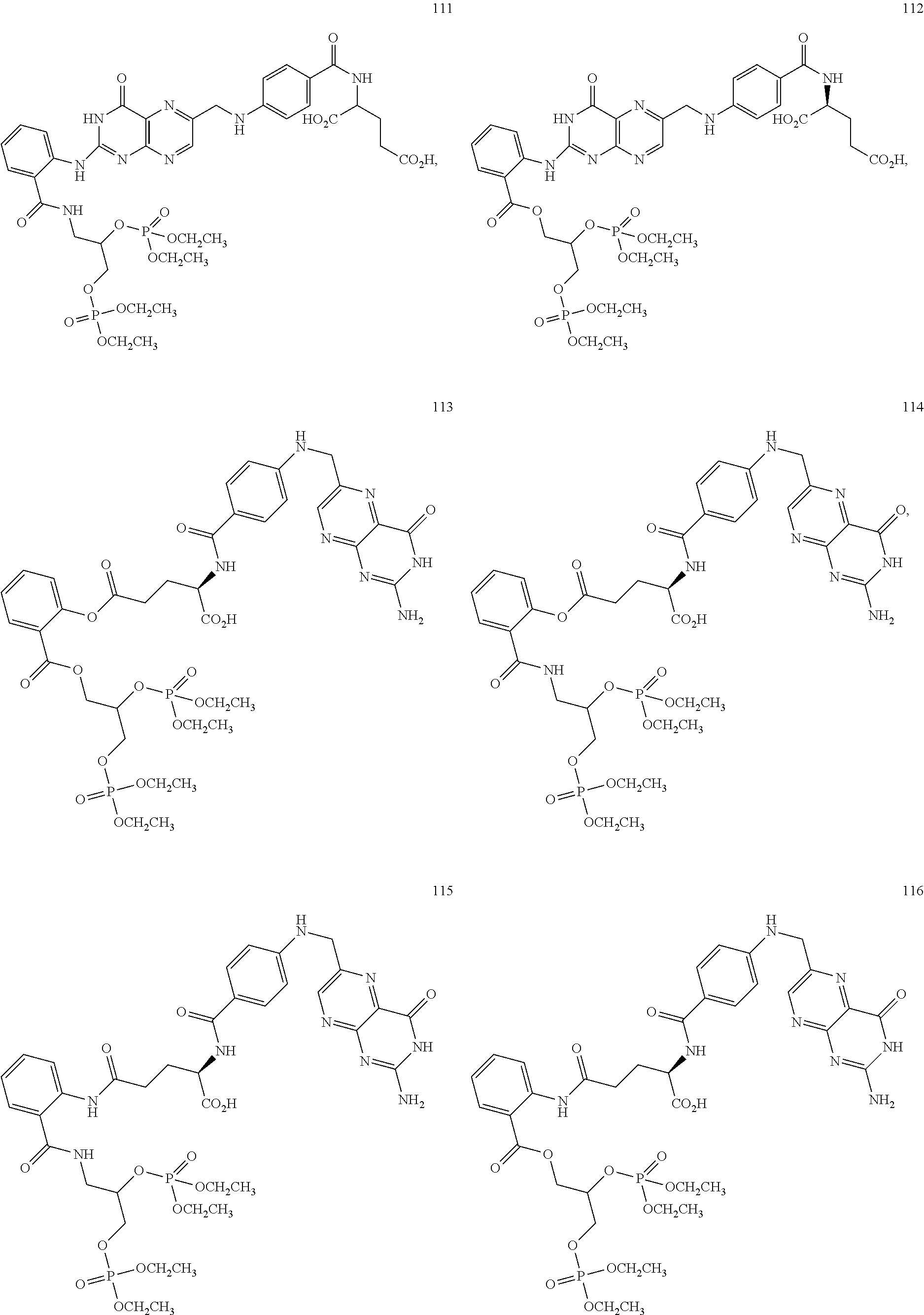

In a fourth aspect the invention features a compound of general Formula IV

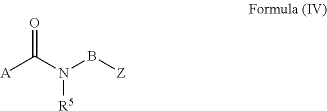

or a pharmaceutically acceptable salt thereof.

In Formula IV: A is an optionally substituted aliphatic, heteroaliphatic, aromatic, heteroaromatic substituent or alkylaryl substituent having 1 to 100 carbon atoms or selected from:

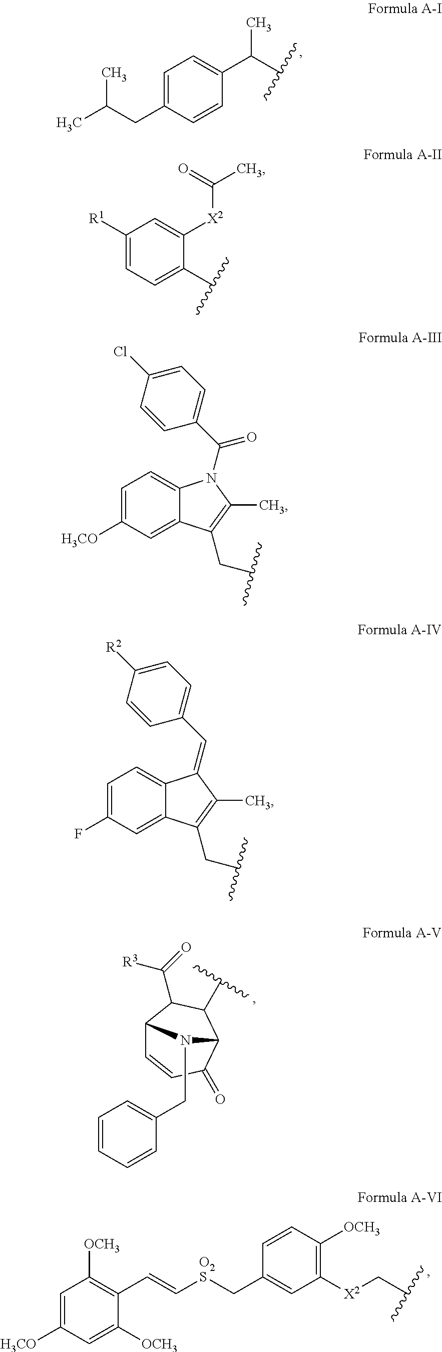

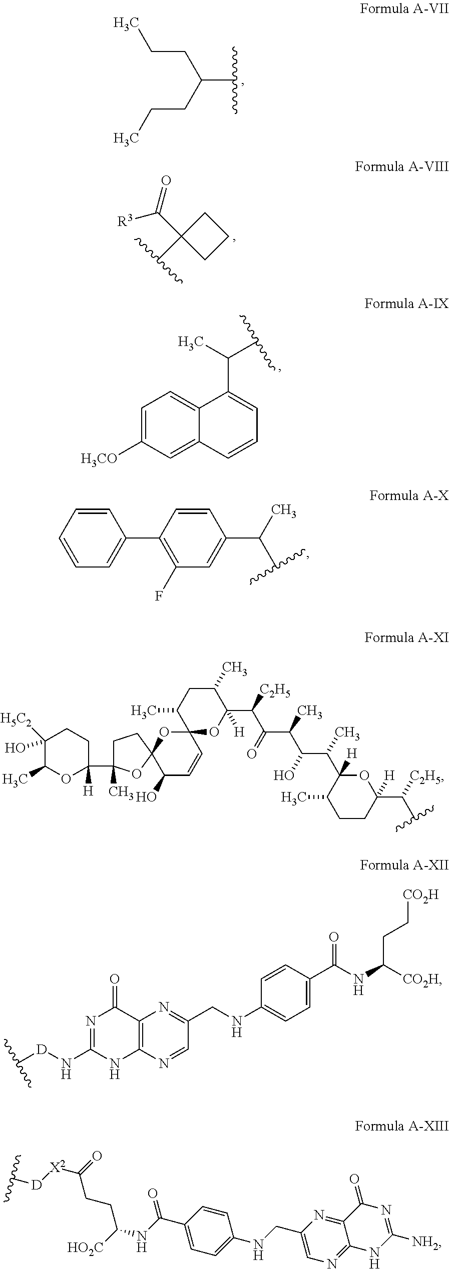

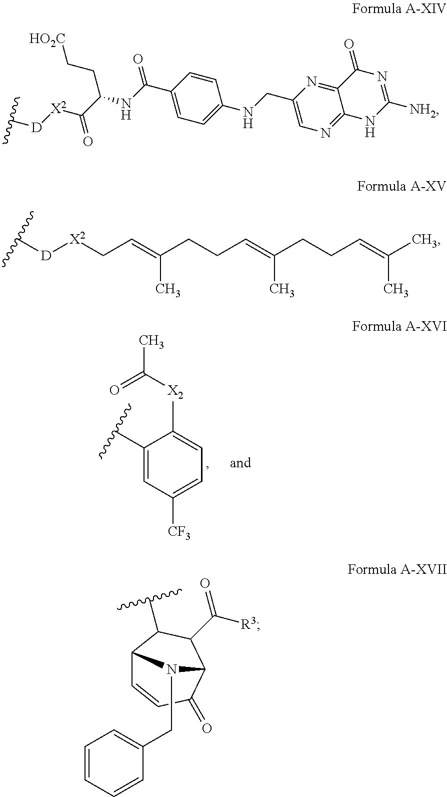

D is absent or

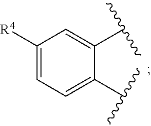

X² is selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B—Z;

R⁵ is selected from methyl and ethyl;

B is selected from:

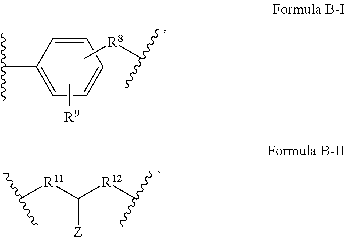

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;

Z is selected from:

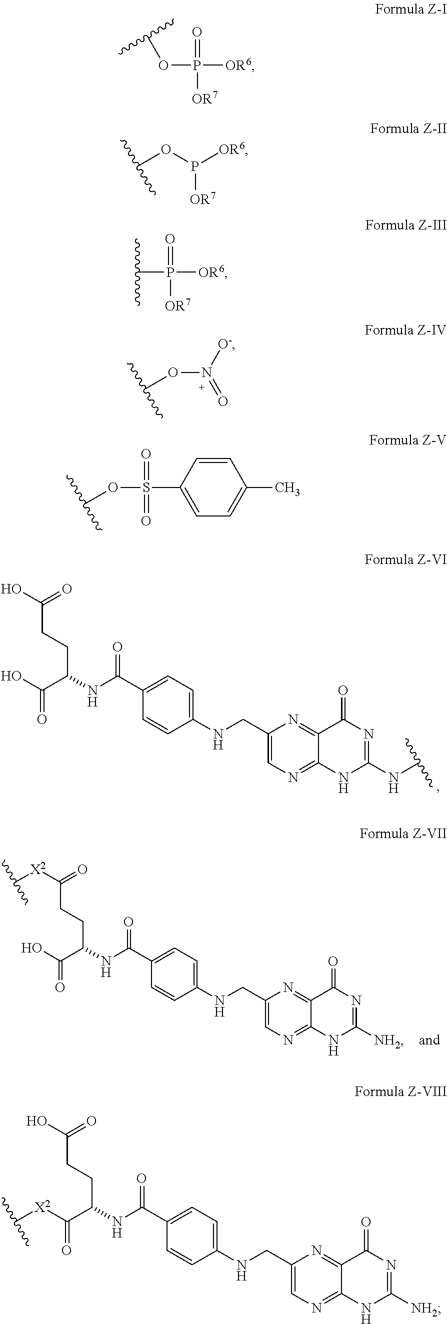

or B together with Z forms a structure:

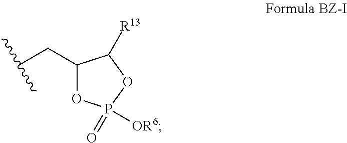

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

In a fifth aspect, the invention features a compound having a structure selected from the group consisting of

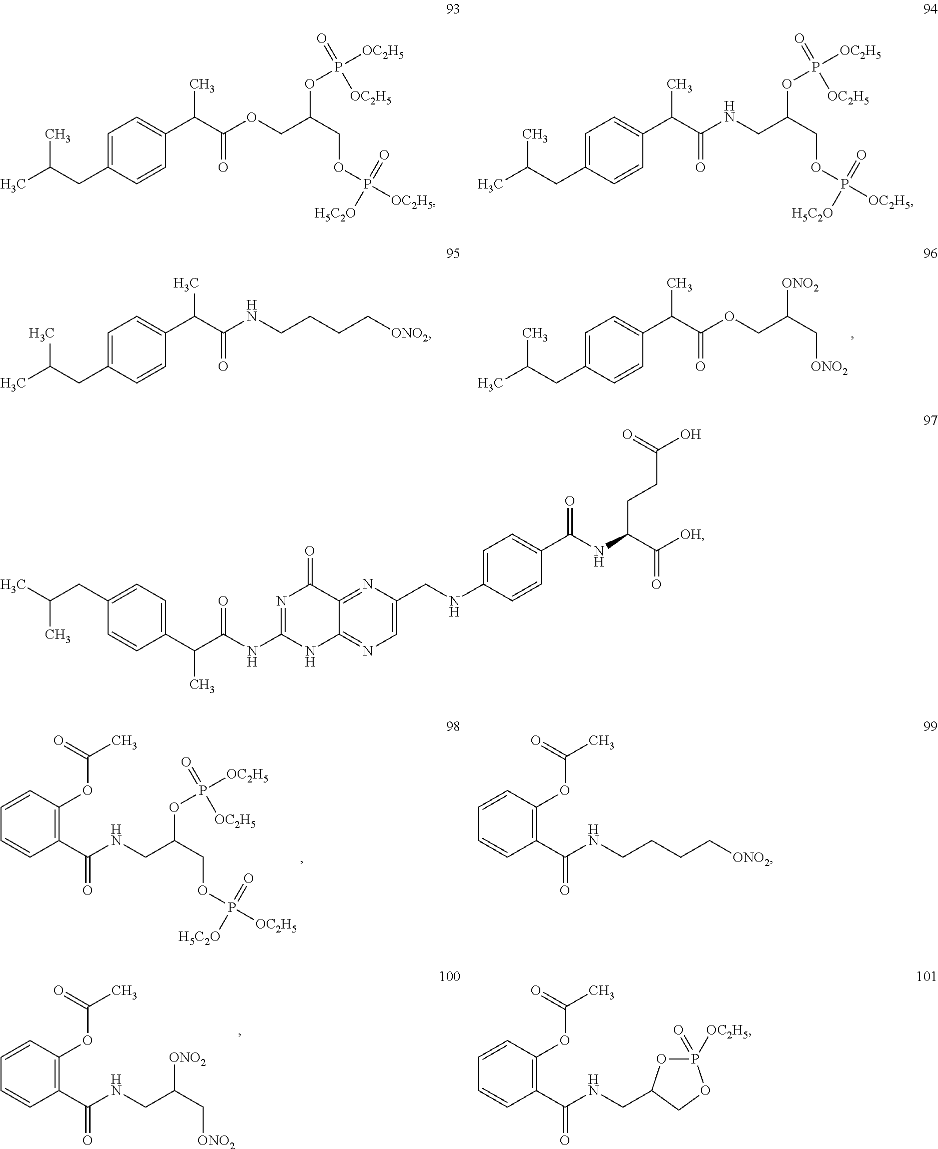

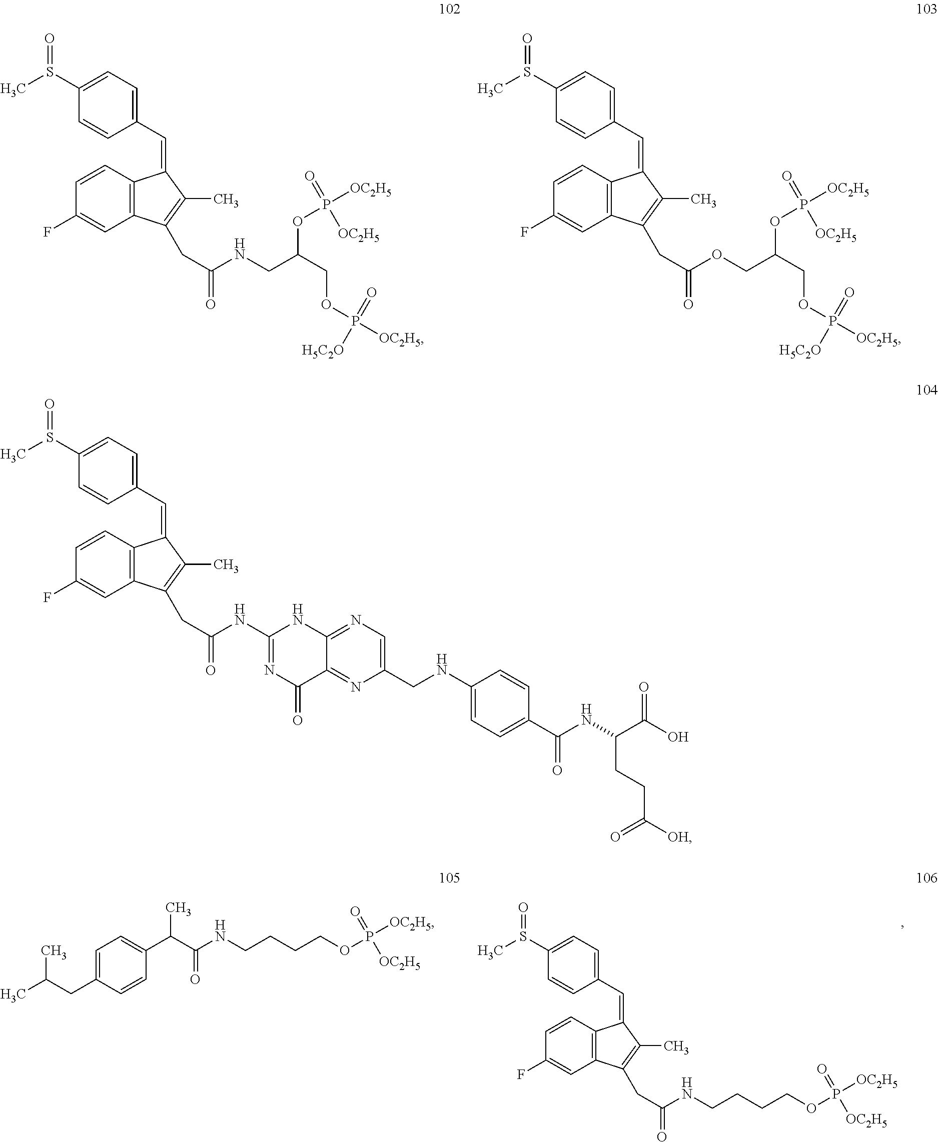

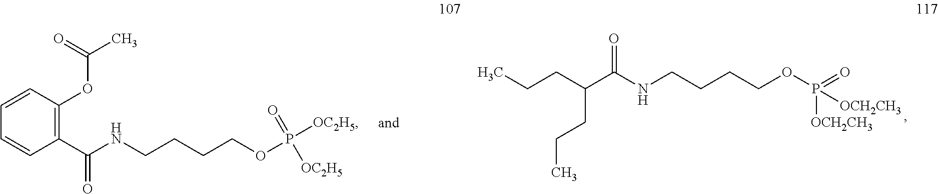 [SMILES: CCOP(=O)(OCC)OCCCCNC(=O)c1ccccc1OC(C)=O ; CCCC(CCC)C(=O)NCCCCOP(=O)(OCC)OCC]

A further aspect of the present invention is directed to a topical pharmaceutical composition comprising a compound of one of Formulas I-IV or any compound specified above, as described generally herein, and a pharmaceutically acceptable excipient.

In a specific embodiment, the composition further comprises difluoromethylornithine or cimetidine.

Another aspect of the present invention relates to the use of an effective amount of compounds represented by Formulas I-IV, any compound specified above or any composition described herein in the treatment of inflammation of a subject in need thereof.

In a specific embodiment, the compound is useful in the treatment of inflammation related to rheumatoid arthritis, Sjogren's syndrome, coronary artery disease, peripheral vascular disease, hypertension, Alzheimer's disease and its variants, lupus erythematosus, chronic bronchitis, chronic sinusitis, benign prostatichypertrophy, prostate cancer, colon adenomas, colon cancer, cancer of the lung, lymphoma, and leukemia.

A further aspect of the present invention relates to the use of an effective amount of compounds represented by Formula I, II, III, or IV, or any specific compound or composition described herein for the treatment or prevention or reduction of the risk of cancer in a subject in need thereof.

In yet another aspect, the present invention features methods for treating cell proliferation by contacting a cell with an effective amount of a compound represented by Formula I, II, III, or IV, or any specific compound or composition described herein.

In a further aspect, the present invention features methods for treating non-cancerous conditions of the skin or mucous membranes, the method including topically administering to a subject in need thereof an effective amount of a compound of Formula V

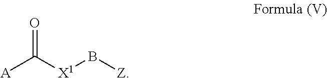

In Formula V: A is an optionally substituted aliphatic, heteroaliphatic, aromatic, heteroaromatic substituent or alkylaryl substituent having 1 to 100 carbon atoms;

X¹ is selected from —O—, —S—, and —NR⁵—;

R⁵ is selected from hydrogen and a C₁₋₆ alkyl;

B is an aliphatic, alicyclic, heteroaliphatic, heterocyclic, aryl, aralkyl, or heteroaromatic group optionally substituted with one or more R¹⁵ moieties,

each R¹⁴ is independently, selected from hydrogen, halogen, hydroxyl, alkoxyl, —CN; an optionally substituted aliphatic, alicyclic, heteroaliphatic, heterocyclic, aryl, aralkyl, heteroaromatic moiety; —OR^{R}, —S(═O)ₙR^{d}, —NR^{b}R^{c}, —C(═O)R^{a} and —C(═O)OR^{a}; n is 0-2; R^{a}, for each occurrence, is independently selected from hydrogen and an optionally substituted aliphatic, alicyclic, heteroaliphatic, heterocyclic, aryl, aralkyl, or a heteroaromatic moiety; each of R^{b} and R^{c}, for each occurrence, is independently selected from hydrogen; hydroxyl, SO₂R^{d}, and aliphatic, alicyclic, heteroaliphatic, heterocyclic, aryl, aralkyl, heteroaromatic or an acyl moiety; R^{d}, for each occurrence, is independently selected from hydrogen, —N(R^{e})₂, aliphatic, aryl and heteroaryl, R^{e}, for each occurrence, is independently hydrogen or aliphatic; and R^{R} is an optionally substituted aliphatic, alicyclic, heteroaliphatic, heterocyclic, aryl, aralkyl, heteroaromatic or acyl moiety;

Z is selected from:

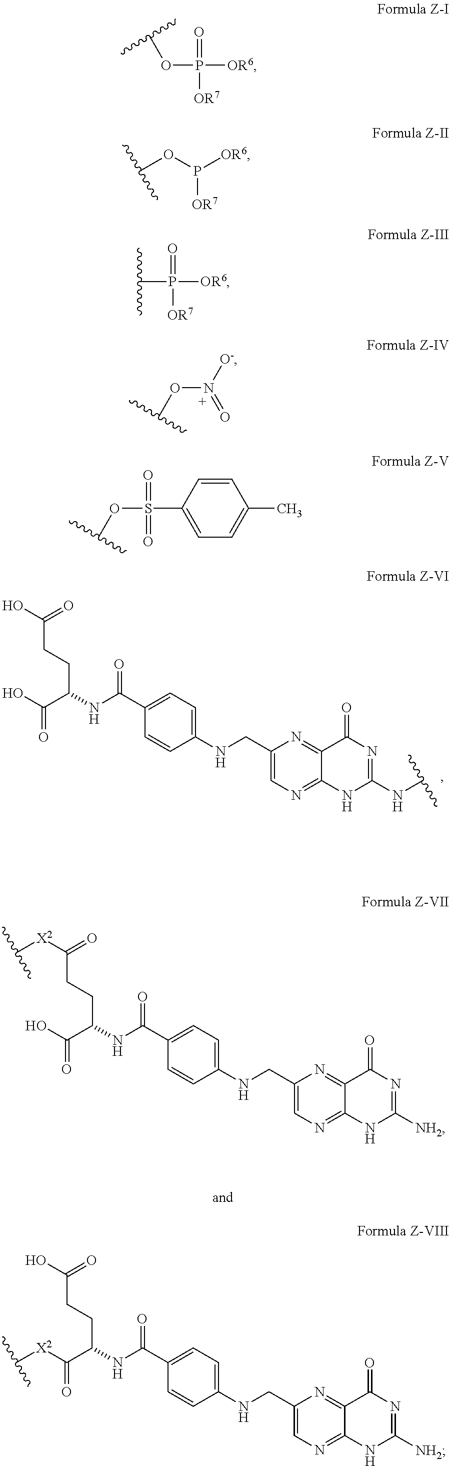

or B together with Z forms a structure:

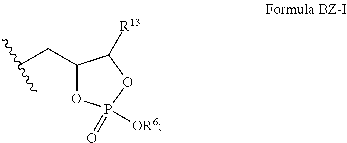

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue;

or a pharmaceutically acceptable salt thereof.

In a specific embodiment, the compound of Formula V is further described by Formula I, II, III, or IV or any specific compound described herein.

In another embodiment the compound of Formula V is a compound disclosed in U.S. Pat. No. 8,236,820, incorporated by reference. For example, the compound of Formula V can be selected from:

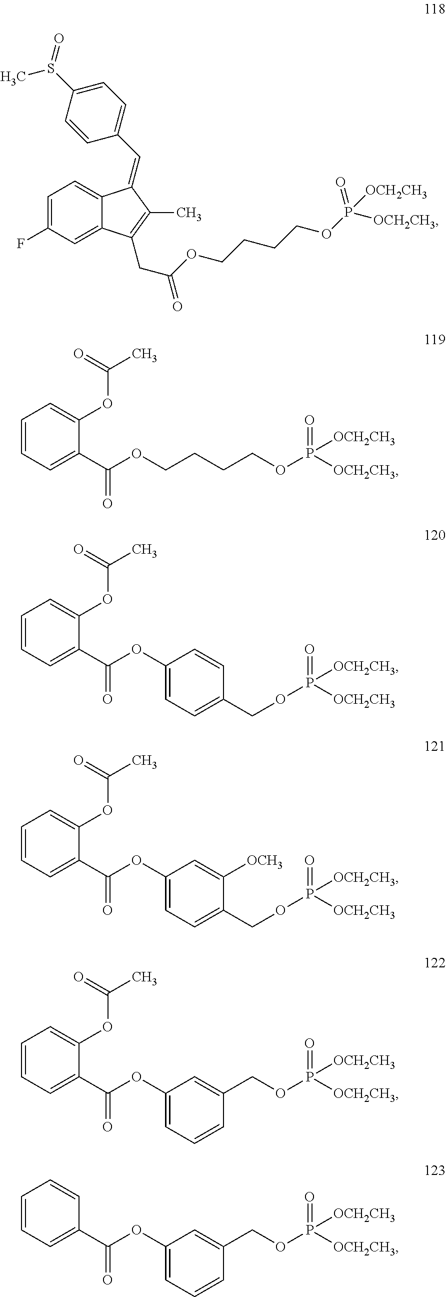 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)c1ccccc1OC(C)=O ; CCOP(=O)(OCC)OCc1ccc(OC(=O)c2ccccc2OC(C)=O)cc1 ; CCOP(=O)(OCC)OCc1ccc(OC(=O)c2ccccc2OC(C)=O)cc1OC ; CCOP(=O)(OCC)OCc1cccc(OC(=O)c2ccccc2OC(C)=O)c1 ; CCOP(=O)(OCC)OCc1cccc(OC(=O)c2ccccc2)c1]

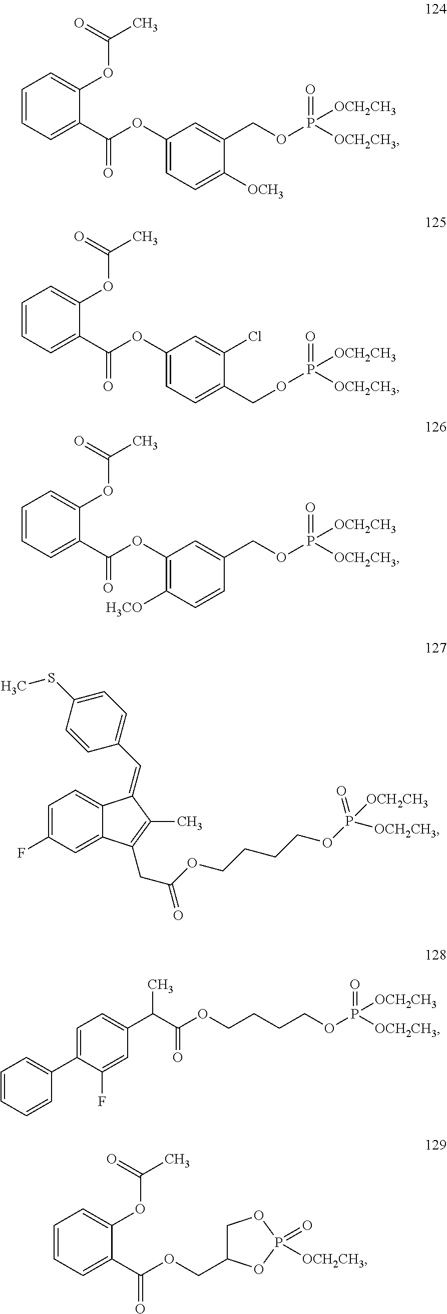 [SMILES: CCOP(=O)(OCC)OCc1cc(OC(=O)c2ccccc2OC(C)=O)ccc1OC ; CCOP(=O)(OCC)OCc1ccc(OC(=O)c2ccccc2OC(C)=O)cc1Cl ; CCOP(=O)(OCC)OCc1ccc(OC)c(OC(=O)c2ccccc2OC(C)=O)c1 ; CCOP(=O)(OCC)OCCCCOC(=O)CC1=C(C)/C(=C/c2ccc(SC)cc2)c2ccc(F)cc21 ; CCOP(=O)(OCC)OCCCCOC(=O)C(C)c1ccc(-c2ccccc2)c(F)c1 ; CCOP1(=O)OCC(COC(=O)c2ccccc2OC(C)=O)O1]

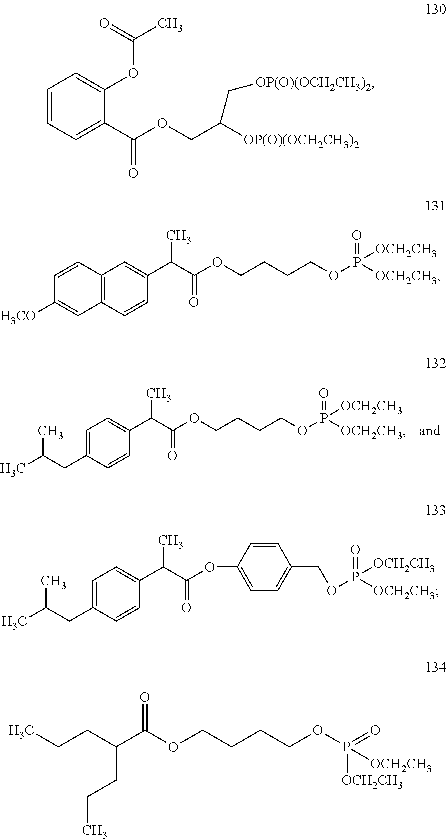

In a further embodiment, the anti-cancer agent comprises a phospho-nonsteroidal anti-inflammatory agent having one or more phosphate moiety (phospho-NSAIDs). The compounds that may be used in the present invention are disclosed in WO 2009/023631, WO 2005/065361, and WO 2011/094589, which are incorporated herein by reference. Further incorporated herein is U.S. provisional application Ser. No. 61/704,021 filed Sep. ______, 2012 titled “COMPOUNDS AND COMPOSITIONS FOR USE IN THE TREATMENT AND PREVENTION OR REDUCTION OF THE RISK OF LUNG AND BRAIN CANCER AND PRECANCEROUS CONDITIONS THEREOF”, which discloses other compounds which may be used herein.

Particularly preferred for this purpose are phospho-ibuprofen I, phospho-ibuprofen glycerol II, phospho-ibuprofen glycerol amide III, phospho-ibuprofen amide IV, phospho-sulindac V, phospho-sulindac amide VI, phospho-aspirin VII, phospho-valproic acid VIII, and the compounds IX and X, the structures of which are shown below:

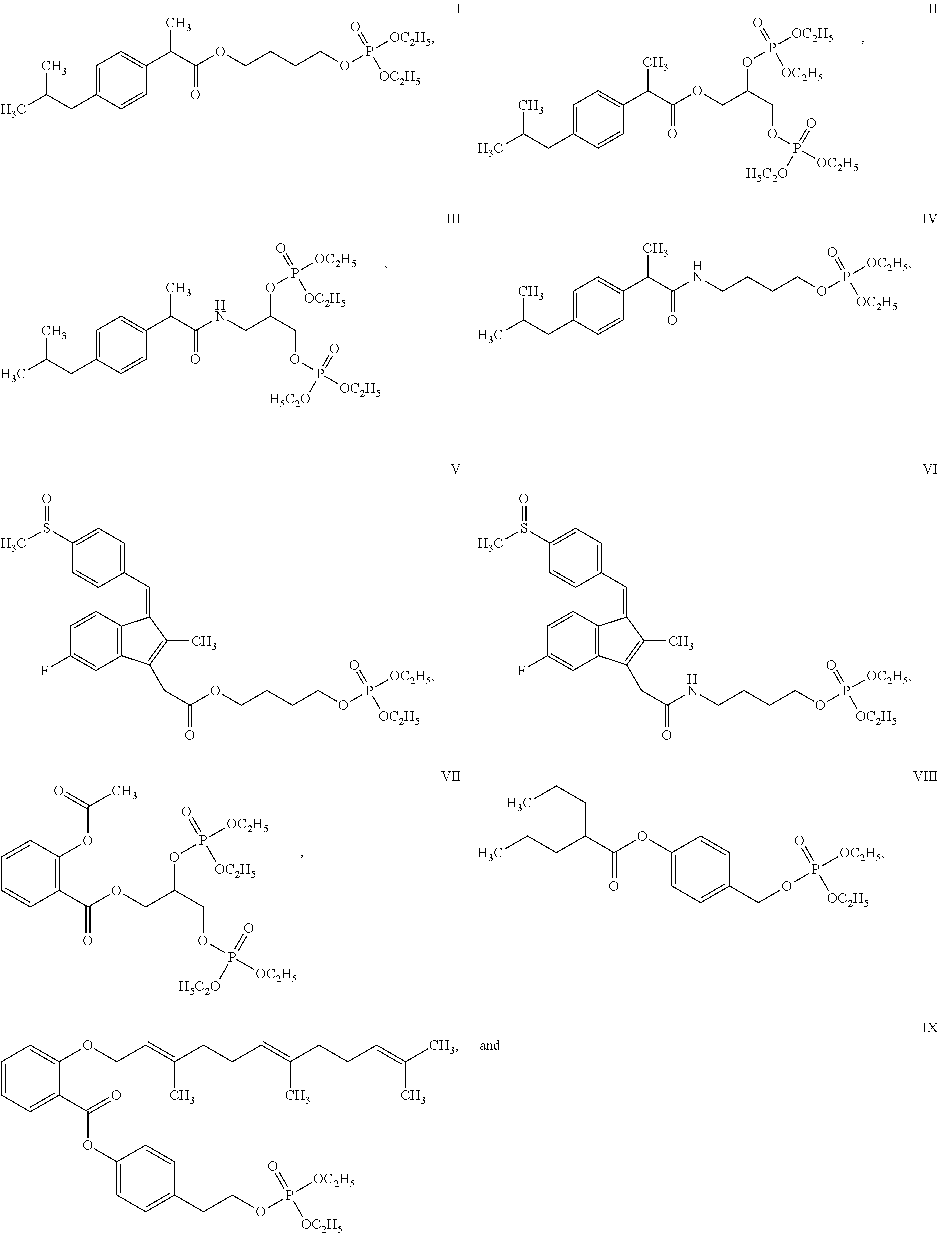

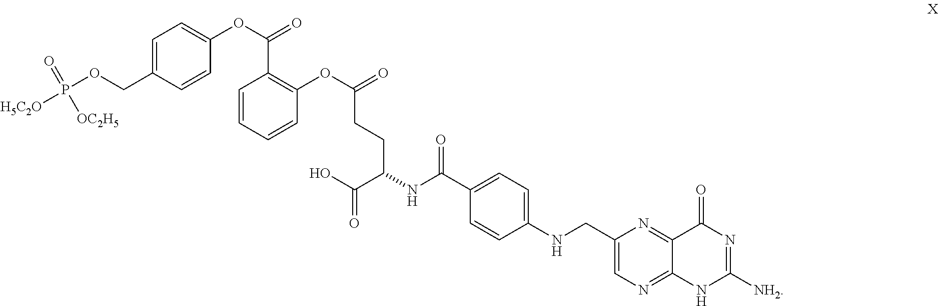

In one embodiment, the anticancer agent in the present invention is a compound having a Formula VI:

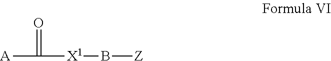

or an enantiomer, a diastereomer, a racemate, a tautomer, salt, hydrate, cocrystal, or compositions thereof,

wherein

X¹ is selected from the group consisting of —O—, —S— and —NR¹—;

R¹ being hydrogen or C₁₋₁₀₀-alkyl, preferably C₁₋₂₂-alkyl, particularly preferred C₁₋₁₀-alkyl;

A is an optionally substituted aliphatic, heteroaliphatic, aromatic, heteroaromatic substituent or alkylaryl substituent having in a preferred embodiment 1 to 100, and even more preferably 1 to 42 carbon atoms. Preferably, A is derived from among NSAIDs. In one of the preferred embodiments, A is selected from the group consisting of:” Yun will redraw the structure

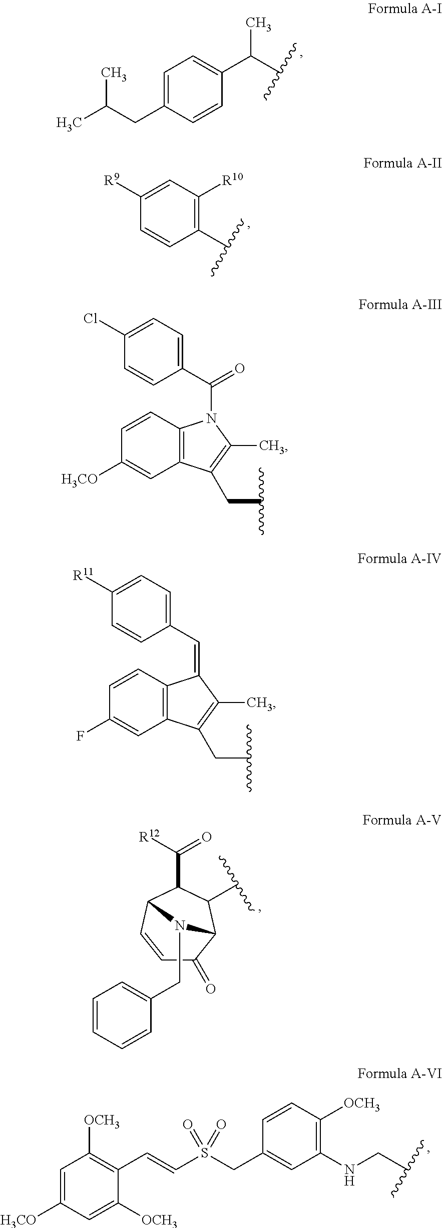

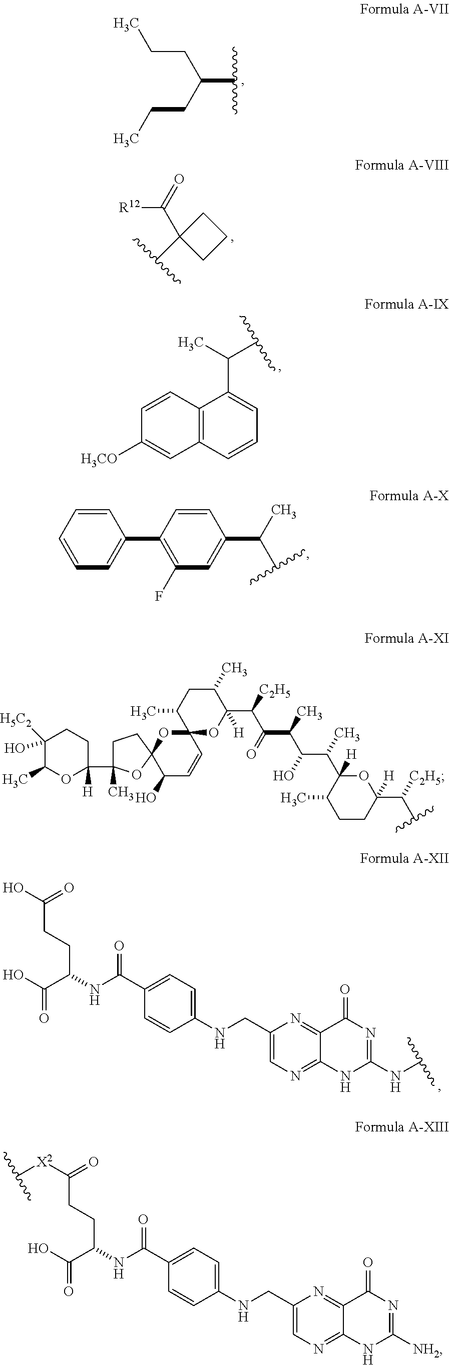

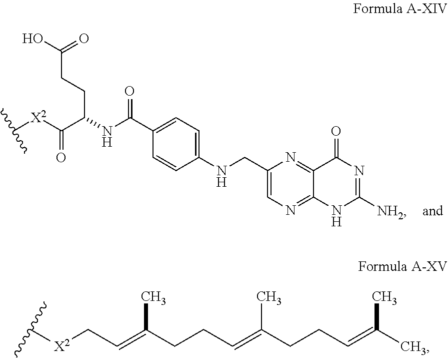

wherein,

R⁹ being selected from hydrogen and trifluoromethyl;

R¹⁰ being selected from —X²—C(O)—CH₃;

R¹¹ being selected from —SCH₃, —S(O)CH₃ and —S(O)₂CH₃;

R¹² being selected from hydroxy, —B—Z and Formula A-XII
whereby
X² is selected from the group consisting of —O—, —S— and —NR¹³—, wherein, R¹³ being hydrogen or C₁₋₆-alkyl;
B is selected from the group consisting of

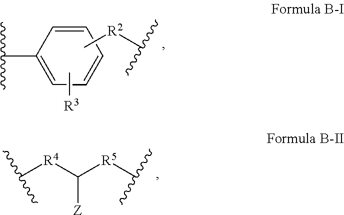

a single bond and an aliphatic substituent, preferably with 1 to 100, more preferred with 1 to 42 and particularly preferred with 1 to 22 carbon atoms,
   wherein,
R², R⁴ and R⁵ being the same or different C₁₋₃-alkylene;
R³ being hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl;
   Z is selected independently from the group consisting of:

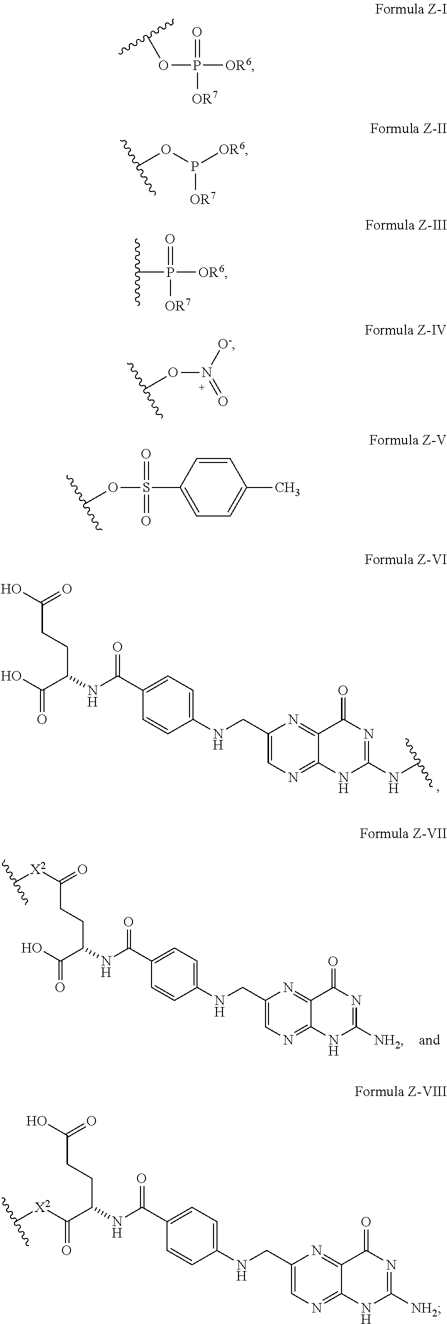

wherein,
R⁶ being independently selected from hydrogen, C₁₋₁₀₀-alkyl, preferably C₁₋₆-alkyl, and polyethylene glycol residue;
R⁷ being selected from hydrogen, C₁₋₁₀₀-alkyl, preferably C₁₋₆-alkyl, and polyethylene glycol residue;

or B together with Z forms a structure:

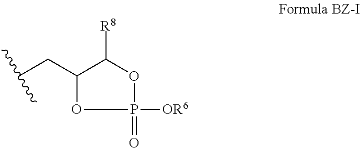

wherein, R⁶ being defined as above; and
R⁸ being independently selected from hydrogen, an aliphatic substituent with 1 to 22 carbon atoms, more preferred C₁₋₆-alkyl and a polyethylene glycol residue

Preferably, the folic acid residue is selected from the group consisting of

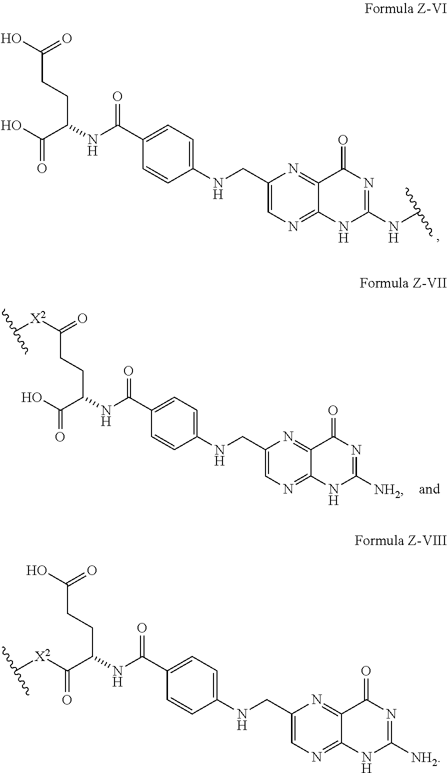

In one embodiment, A is represented by Formula A-I or A-IV, X1 is —O— and —B—Z is not —(CH₂)₄—O—P(O)(OC₂H₅)₂.

In another embodiment, A is represented by Formula A-II and X1 is not —O— and/or —B— is an aliphatic substituent with 1 to 100, preferably with 1 to 42 carbon atoms.

In another embodiment, the anticancer agent in the present invention is selected from compounds having Formula (VII)

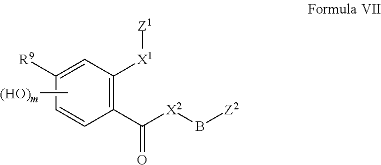

or an enantiomer, a diastereomer, a racemate, a tautomer, salt or hydrate thereof,

wherein,

m=0 or 1;

X¹ and X² are independently selected from the group consisting of —O—, —S— and —NR¹—, R¹ being hydrogen or C₁₋₆-alkyl;

B is an optionally substituted aliphatic, heteroaliphatic, aromatic, heteroaromatic or alkylaryl substituent having 1 to 40 carbon atoms;

Z¹ is selected from the group consisting of hydrogen, farnesyl and a folic acid residue;

Z² is selected from the group consisting of

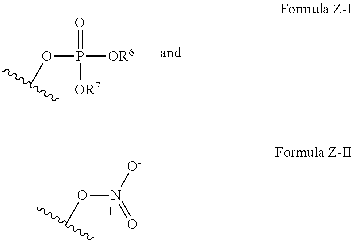

whereby
Z² is preferably represented by Formula Z-I;
R⁶ being independently selected from hydrogen, C₁₋₁₀₀-alkyl and a polyethylene glycol residue,
R⁷ being independently selected from hydrogen, C₁₋₁₀₀-alkyl and a polyethylene glycol residue; or
B together with Z² forms a structure

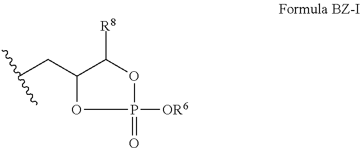

R⁶ being defined as above, and
R⁸ being independently selected from hydrogen, an aliphatic substituent with 1 to 22 carbon atoms, more preferred C₁₋₆-alkyl and a polyethylene glycol residue and
R⁹ being selected from hydrogen and trifluoromethyl.

A further aspect of the present invention, the anticancer agent relates to the compounds of Formula VIII:

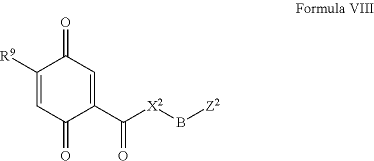

or an enantiomer, a diastereomer, a racemate, a tautomer, salt or hydrate thereof, wherein X², B, Z² and R⁹ are as defined above.

The choice of the nicotine-containing material for use in the product of the present invention is not particularly limited. Preferably, the nicotine-containing material used is the leaf of a tobacco plant i.e. a plant of the genus Nicotiana, such as Nicotiana tabaccum. Tobacco leaves of several types may be employed. Suitable types of tobacco leaves include, but are not limited to, Brightleaf tobacco, Burley, Cavendish, Corojo, Criollo, Oriental tobacco, Perique, Shade tobacco, Thuoc lao, Type 22, White Burley, wild tobacco and Y1.

The content of the nicotine-containing material and of the anti-cancer agent in the product can be readily chosen by a person skilled in the art to provide an advantageous therapeutic effect. Preferably, the product contains nicotine and the anti-cancer agent in the ratio of from 1000:1 to 1:10, more preferred from 10:1 to 1:10, even more preferred from 7:1 to 1:7, particularly preferred from 4:1 to 1:4, for instance 1:1 (weight:weight).

Alternatively, the nicotine-containing material may be nicotine (IUPAC name: (S)-(−)-3-(1-methylpyrrolidin-2-yl)pyridine) or a pharmaceutically acceptable salt thereof. S. M. Berge et al. describe pharmaceutically acceptable salts in detail in J. Pharmaceutical Sciences, 1977, 66, 1-19, incorporated herein by reference. Examples of pharmaceutically acceptable acid addition salts can be formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts and coformer molecules for cocrystal formation include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Nicotine may be further bound to a polymeric material such as an ion-exchange resin, for instance to polymethacrilic acid, such as Amberlite® IRP64. The corresponding material is commercially available under the name Nicotine Polacrilex.

Preferably, one dosage of the product contains nicotine from 0.01 to 100 mg, preferably from 0.1 to 10 mg, more preferred from 0.5 to 7 mg, yet even more preferred from 0.7 to 5 mg and particularly preferred from 1 to 3 mg nicotine. Depending on the intended mode of administration, the product of the present invention may additionally comprise a pharmaceutically acceptable carrier which, as used herein, includes solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Some examples of materials which can serve as pharmaceutically acceptable carriers include, but are not limited to, volatile solid materials, such as menthol, sugars such as lactose, glucose and sucrose; excipients such as cocoa butter; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and DSPE-PEG2000, phosphatidic acid, phosphatidyl glycerol and phosphatidyl serine. Commercial grades of lecithin which are preferred include those which are available under the trade name Phosal® or Phospholipon® and include Phosal® 53 MCT, Phosal® 50 PG, Phosal® 75 SA, Phospholipon® 90H, Phospholipon® 90G and Phospholipon® 90 NG; soy-phosphatidylcholine (SoyPC) and DSPE-PEG2000 are particularly preferred; buffering agents such as amino acids; pyrogen-free water; isotonic saline; Ringers solution; ethyl alcohol, and phosphate buffer solutions, as well as other non-toxic compatible lubricants such as sodium lauryl sulfate as well as releasing agents, coating agents, sweetening, flavoring and perfuming agents, preservatives and antioxidants can also be present in the product of the present invention.

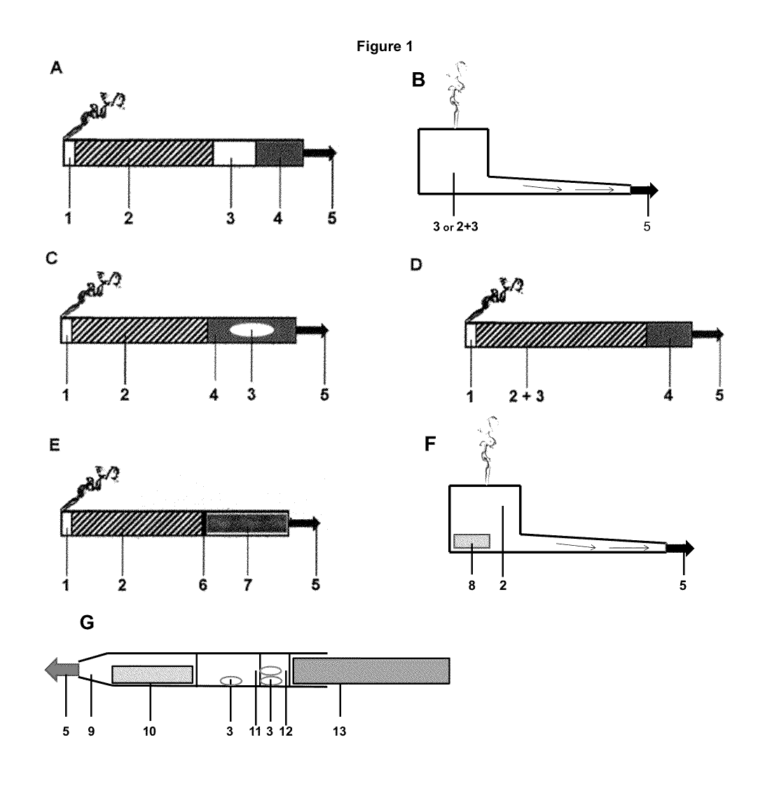

In some embodiments, the product for the present invention is a smoking device such as cigarette, cigarette holder, cigar or smoking pipe. In these embodiments, the anti-cancer agent is inhaled at the same time that the smoker smokes. For this purpose, the nicotine-containing material and the anti-cancer agent can be, for instance, incorporated in a cigarette, a cigar (see FIG. 1A) or in a smoking device such as a smoking pipe (for instance, in the chamber of a smoking pipe, see FIG. 1B) or in a water pipe, etc. In addition, the smoking device may optionally comprise an additional unit which renders the anti-cancer agent suitable for inhalation so that smoke and the anti-cancer agent can be inhaled simultaneously or in sequence. The additional unit may, for instance, be a pressurized aerosol spray dispenser, a nebulizer, an atomizer or a cartonizer.

The term “smoking” as used herein refers to the action of inhaling or tasting the smoke of burning plant material, preferably of tobacco leaves, and also includes a process wherein the smoking composition.

FIG. 1A: the anti-cancer agent or a pharmaceutical composition thereof 3 is incorporated into the cigarette containing tobacco 2 and, optionally, having a filter 4. Tobacco smoke coming from the pyrolysis zone 1 or in a close distance causes volatilization of the anti-cancer agent. In order to improve volatilization the anti-cancer agent can be formulated with a volatile solid such as menthol. The tobacco smoke 5 containing the anticancer agent enters the mouth and the lungs of the smoker.

FIG. 1B: the anticancer agent or a pharmaceutical composition thereof 3 is incorporated into a smoking pipe. Tobacco 2 may be mixed with the anticancer agent or a pharmaceutical composition thereof 3. Alternatively, another smoking device such as a water pipe can be employed. The volatilization of the anti-cancer agent can be additionally facilitated by external heating, for instance, by using an electric heating element.

FIG. 1C: a further embodiment of the present invention is shown. The anti-cancer agent is administered in a so-called “cigarette with menthol capsule”. The anti-cancer agent or a pharmaceutical composition thereof 3 is incorporated in a menthol capsule which, in turn, is located in the filter 4. Cigarettes with menthol capsules are known in the prior art and are, for example, described in US 2009/0277465. The anticancer agent or a pharmaceutical composition thereof is incorporated into the menthol capsule and is volatilized during the smoking process. Thus, this embodiment is particularly suited for smokers and aims to prevent lung cancer and/or precancerous conditions in the lung.

FIG. 1D: a further embodiment is shown. The anti-cancer agent or a pharmaceutical composition thereof 3 is directly mixed with tobacco 2. Thus, volatilization the anti-cancer agent occurs primarily in the pyrolysis zone 1 of the cigarette and the tobacco smoke 5 containing the anti-cancer agent enters the mouth and the lungs of the smoker. In this embodiment, the filter 4 is optional. This embodiment is particularly useful if the anti-cancer agent is sufficiently volatile.

FIG. 1E: a further embodiment is shown. The anti-cancer agent or a pharmaceutical composition thereof 3 (not shown) is incorporated in an electronic cigarette cartridge 7. The cartridge 7 may be designed as an atomizer or as a cartonizer. Valve 6 prevents the entry of the aerosol and solvent vapor emitted by the cartridge 7 into the tobacco section 2. In this embodiment, tobacco smoke formed in the pyrolysis zone 1 enters the section containing the electronic cigarette cartridge 7 via the valve 6. Thus, the aerosol emitted by the electronic cigarette cartridge 7 is mixed with the tobacco smoke and the resulting mixture 5 is subsequently inhaled by the smoker.

FIG. 1F: a further embodiment is shown. The anti-cancer agent or a pharmaceutical composition thereof 3 (not shown) is incorporated in an additional unit 8 which may be an atomizer or cartonizer or similar device that renders the anti-cancer agent suitable for inhalation. Having an appropriate valve or other mechanism(s), smoke and inhalable agent may be mixed to simultaneously deliver smoke and anti-cancer agent to the mouth and ultimately the lungs of the smoker.

FIG. 1G: a further embodiment is shown. A cigarette holder has a receptacle for the cigarette 13, an area where the anti-cancer agent or a pharmaceutical composition thereof 3 is stored in an additional unit 12, from which is can be transferred to a compartment 11, where an atomizer or cartonizer or similar device renders the anti-cancer agent suitable for inhalation. The function of this cigarette holder is powered by a battery 10. Having an appropriate arrangement that allows suitable communication of the various compartments of this device, smoke and inhalable agent are mixed to deliver, through a mouthpiece 9, smoke 5 containing the anti-cancer agent to the mouth and ultimately the lungs of the smoker.

In a further embodiment one or more anti-inflammatory agents are used in the place of the anti-cancer agents. In a further yet embodiment a combination of both anti-inflammatory and anti-cancer agents is used.

In all embodiments shown in FIGS. 1A-1G the smoker also inhales the anti-cancer agent during inhalation of the tobacco smoke. In order to facilitate volatilization of the anti-cancer agent, it can be formulated in a dry powder aerosol composition such as the one described by Plumley C, et al. (Int. J. Pharm. 369, (1-2), pages 136-143, 2009) (incorporated by reference herein) or in a pharmaceutical composition containing volatile solids such as menthol. Alternatively, the neat anti-cancer agent can be used instead of the pharmaceutical composition thereof.

In other embodiments, the product of the present invention is a smoking cessation product such as, for instance, transdermal patch, inhalation device, orally applied product or rectal suppository.

In one embodiment of the present invention, the product is a transdermal patch. Transdermal patches comprising a nicotine-containing material are known in the prior art and are, for instance, described in US 2009/0246264, which is incorporated herein by reference. Preferably, the transdermal patch simultaneously delivers nicotine and the anti-cancer agent to the patient. According to the present invention, said transdermal patch preferably releases more than 30 wt.-%, more preferably more than 50 wt.-% and particularly preferred more than 70 wt.-% of its total content of the anti-cancer agent within 24 h to the skin of the patient. The nicotine-containing material and the anti-cancer agent may be present in separate layers of the transdermal patch or as a mixture in the same layer. The layers containing the nicotine-containing material, the anti-cancer agent or a mixture thereof typically contain gelling agents such as homo- or copolymers of 2-hydroxyethyl acrylate or 2-hydroxyethyl methacrylate, poly(vinyl alcohol), Pluronic®, carboxymethyl cellulose, hydroxyethyl starch, hydroxypropyl cellulose or methyl cellulose. These layers may further contain suitable penetration enhancers such as dimethyl sulfoxide, N,N-dimethylacetamide, triglycerides (e.g. soybean oil), unsaturated oils, aloe compositions (e.g. aloe vera gel), octalylphenylpolyethylene glycol, oleic acid, polyethylene glycol 400, propylene glycol, n-decyl methyl sulfoxide, fatty acid esters (e.g. isopropyl myristate, methyl laurate, glycerol monooleate and propylene glycol monooleate) and N-methylpyrrolidone or mixtures thereof.

In another embodiment of the present invention, the product is an inhalation device. The inhalation device may be a smoking device, a mechanical device for pulmonary delivery, a device for the nasal anti-cancer agent delivery or a so-called electronic cigarette. Mechanical devices for pulmonary delivery of the nicotine-containing material and the anti-cancer agent include, but are not limited to, nebulizers, metered dose inhalers, and powder inhalers, all of which are familiar to those skilled in the art. Some specific examples of commercially available devices suitable for the practice of this invention are the Ultravent nebulizer (Mallinckrodt, Inc., St. Louis, Mo., USA), the Acorn II® nebulizer (Marquest Medical Products, Englewood, Colo., USA), the Ventolin® metered dose inhaler (Glaxo Inc., Research Triangle Park, N.C. USA) and the Spinhaler powder inhaler (Fisons Corp, Bedford, Mass. USA).

Devices for nasal anti-cancer agent delivery are also known to persons skilled in the art and are commercially available, for instance, from Bespak (Bespak Europe Limited, United Kingdom).

In some other embodiments, the pharmaceutical composition of the present invention is directly heated, whereby nicotine and the anti-cancer agent form a vapor and subsequently condense into an aerosol. Thus, an aerosol containing nicotine and the anti-cancer agent is formed. Subsequently, the patient inhales this aerosol. Suitable devices are known in the prior art and are, for instance, described in US 2003/0000518.

Alternatively, the combination of the nicotine-containing material and the anti-cancer agent may be administered in a so-called electronic cigarette. Such devices are known in the prior art and are, for instance, described in US 2006/0196518, US 2007/0267031 and Caponnetto et al. (Journal of Medical Case Reports 5, 585, 2011). An electronic cigarette is primarily used for the administration of nicotine and, optionally, of flavors such as menthol. Incorporating of the nicotine-containing material and the anti-cancer agent in the cartridge allows their efficient administration by the respiratory route. Advantageously, said cartridge can be employed in a commercially available electronic cigarette. The cartridge may be an atomizer or a cartonizer, as known in the prior art.

Accordingly, in a further embodiment of the present invention, the product is a cartridge comprising the nicotine-containing material and the anti-cancer agent for use in an electronic cigarette. Such cartridge can also be used by patients suffering from lung cancer or those with precancerous conditions of the lung.

The smoking cessation product may be in the form of a pressurized aerosol spray dispenser, which contains a suitable propellant, e.g. hydrofluoroalkanes, chlorofluorocarbons, carbon dioxide, or a nebulizer. In this embodiment, the dosage unit may be determined by providing a valve to deliver a metered amount. Capsules and cartridges of e.g. gelatine for use in an inhaler or insufflator may be formulated containing a powder mix of the nicotine-containing material, the anti-cancer agent and a suitable pharmaceutically acceptable carrier.

Administration by the respiratory route usually requires the use of pharmaceutical compositions suitable for the dispensing of the nicotine-containing material and the anti-cancer agent. Typically, each pharmaceutical composition is specific to the type of device employed and may involve the use of an appropriate propellant material, in addition to the usual diluents, adjuvants and/or carriers. Also, the use of liposomes, microcapsules or microspheres, inclusion complexes, micelles or other anti-cancer agent nanocarriers, or other types of carriers is contemplated. The combination of the nicotine-containing material and the anti-cancer agent may be prepared in different pharmaceutical compositions depending on their physical and chemical properties or the type of device employed.

Pharmaceutical composition suitable for use with a nebulizer, either jet or ultrasonic, will typically comprise the nicotine-containing material, preferably nicotine or a pharmaceutically acceptable salt thereof, and the anti-cancer agent dissolved in a solvent and containing typically about 0.1 to 25 mg of the anti-cancer agent per 1 ml of solution. The pharmaceutical composition may also include a buffer, for instance, an amino acid, and a simple sugar (e.g. for stabilization of the anti-cancer agent and regulation of osmotic pressure). The solvent in the pharmaceutical composition may be selected from the group consisting of water, ethanol, 1,3-propylene glycol, glycerol or a mixture of any of those. Nebulized pharmaceutical compositions may also contain a surfactant, to reduce or prevent surface induced aggregation of the nicotine-containing material or of the anti-cancer agent caused by atomization of the solution in forming the aerosol. Pharmaceutical compositions for use with a metered-dose inhaler device generally comprise a finely divided powder containing the nicotine-containing material and the anti-cancer agent (or a pharmaceutically acceptable derivative thereof) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Pharmaceutical compositions for dispensing from a powder inhaler device will comprise a finely divided dry powder containing the nicotine-containing material and the anti-cancer agent and may also include a bulking agent, such as lactose, sorbitol, sucrose, or mannitol in amounts which facilitate dispersal of the powder from the device, e.g. 50 to 90% by weight of the formulation. The nicotine-containing material and the anti-cancer agent should most advantageously be prepared in a particulate form with an average particle size of less than 10 μm, preferably less than 5 μm and more preferred less than 1 μm, for effective delivery to the distal lung.

In a further embodiment of the present invention, the product is a rectal suppository. In this embodiment, the nicotine-containing material and the anti-cancer agent are mixed with suitable non-irritating excipients or carriers such as cacao butter, polyethylene glycol or a suppository wax, which are solid at ambient temperature but liquid at body temperature and therefore melt in the rectum cavity and release nicotine and the anti-cancer agent.

In yet a further embodiment of the present invention, the product is an orally applied product. Thus, for instance, the product may be in the form of a chewing gum. Nicotine chewing gums are known in the prior art, are described in US 2010/0130562, incorporated by reference herein and are commercially available under the trade names such as Nicorette® and Thrive®. In this embodiment, the product of the present invention contains the nicotine-containing material, the anti-cancer agent as well as the chewing gum base, plasticizers, buffering agents, sweeteners, antioxidants, flavoring agents and colorants. Examples of suitable plasticizers include lecithin, lanoline, glycerides, stearic acid, sodium stearate, potassium stearate or waxes such as bee wax. Examples of sweeteners that may be used in the product of the present invention include saccharides as well as salts of saccharine or cyclamic acid as well as sugar alcohols such as sorbitol, mannitol, or xylitol. The flavoring agents for use in the product of the present invention may include, without limitation, the flavors of cherry, cinnamon, grape, apple, lemon, orange, peppermint, raspberry, strawberry, chocolate, and the like.

In a further embodiment, the orally applied product is in the form of smokeless tobacco. In this embodiment, the smokeless tobacco is formulated with the anti-cancer agent and further contains plasticizers as well as sweeteners and flavoring agents described above. Smokeless tobacco products include, but are not limited to, dipping tobacco, chewing tobacco, snuff, snus, creamy snuff, tobacco gum, gutkha, gul, khaini, qiwam, mawa, mishri, pan masala and zarda, chewing tobacco being particularly preferred.

Another aspect of the present invention relates to the products described herein for use in the treatment and/or prevention or reduction of the risk of cancer and/or precancerous conditions. “Cancer” as used herein refers to an uncontrolled growth of cells which interferes with the normal functioning of the bodily organs and systems. Cancers include, but are not limited to, basal cell carcinoma, biliary tract cancer, bladder cancer, bone cancer, brain and other central nervous system (CNS) cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemias, including hairy cell leukemia, liver cancer, lung cancer (e.g. small cell and non-small cell), lymphomas including Hodgkin's and non-Hodgkin's lymphomas, melanoma, myeloma, neuroblastoma, oral cavity cancer (e.g. lip, tongue, mouth, and pharynx), ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, as well as other carcinomas and sarcomas.

In yet another embodiment, the product of the present invention is useful in the treatment and/or prevention or reduction of the risk of cancer and precancerous conditions, including, but not limited to, benign prostatic hypertrophy, colon adenomas, actinic keratosis and various premalignant conditions of the lung, breast and pancreas.

The anti-cancer agent and pharmaceutical compositions thereof inhibit the growth of human or animal cancer cell lines such as A549 human lung cancer cells in in vitro tests and have IC50 value of preferably less than 800 μM, more preferred of less than 400 μM, particularly preferred of less than 70 μM. The tests are preferably carried out as specified in S. Joseph et al. (Molecular Medicine Reports 2011, 4, 891-899).

One embodiment of the present invention relates to a method for preventing or reducing the risk of cancer by means of administering the product of the present invention. Accordingly, treatment of an individual with the product of the present invention reduces the risk of the individual to develop cancer. Preferably, after the treatment, the risk of the individual to develop cancer is reduced by 5% or greater; more preferably, the risk develop cancer is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. As used herein, reducing risk of developing cancer includes decreasing the probability or incidence of developing cancer for an individual compared to a relevant, e.g. untreated, control population, or in the same individual prior to treatment according to the invention. Reduced risk of developing cancer may include delaying or preventing or reducing the risk of the onset of a cancer. Risk of developing cancer can also be reduced if the severity of a cancer or a precancerous condition is reduced to such a level that it is not of clinical relevance. That is, the cancer or a precancerous condition may be present but at a level that does not endanger the life, activities, and/or well-being of the individual. For example, a small tumor may regress and disappear, or remain static. Preferably, tumor formation does not occur. In some circumstances the occurrence of the cancer or the precancerous condition is reduced to the extent that the individual does not present any signs of the cancer or the precancerous condition during and/or after the treatment period.

The method for preventing or reducing the risk of cancer according to the present invention is beneficial both for individuals having a precancerous condition and individuals who are healthy. Individuals with lifestyle habits that could lead to cancer, particularly smokers, and individuals affected by diseases for which the probability of cancer incidence is high have a particularly high order of priority as individuals for the preventive method of the present invention. Furthermore, individuals who are likely to acquire familial cancers, and such individuals as those who are diagnosed with a risk of cancer by means of gene diagnoses based on single-nucleotide polymorphism or the like may also be targeted.

Treating cancer can result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may further result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%.

Treating and/or preventing or reducing the risk of cancer can result in an increase in average survival time of a population of individuals treated according to the present invention in comparison to a population of untreated individuals. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with the product of the present invention. An increase in average survival time of a population may also be measured, for example, by calculating for a population the average length of survival following completion of a first round of treatment with the product of the present invention.

Treating and/or preventing or reducing the risk of cancer can also result in a decrease in the mortality rate of a population of treated individuals in comparison to an untreated population. Preferably, the mortality rate is decreased by more than 2%; more preferably, by more than 5%; more preferably, by more than 10%; and most preferably, by more than 25%. A decrease in the mortality rate of a population of treated individuals may be measured by any reproducible means, for example, by calculating for a population the average number of disease-related deaths per unit time following initiation of treatment with the product of the present invention. A decrease in the mortality rate of a population may also be measured, for example, by calculating for a population the average number of disease-related deaths per unit time following completion of a first round of treatment with the product of the present invention.

A further embodiment of the present invention relates to a method for preventing or reducing the risk of cancer recurrence by means of administering the product of the present invention. Cancer recurrence is a re-development of the cancer in an individual, who had previously undergone a cancer treatment, after a period of time in which no cancer could be detected. The probability of a cancer recurring depend on many factors, including the type of cancer and its extent within the body at the time of the treatment.

Some embodiments of the present invention are directed to the prevention or reduction of the risk of and/or treatment of lung cancer.

Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), non-squamous non-small cell lung cancer, squamous non-small cell lung cancer, squamous cell carcinoma, non-squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructual heterogeneity (e.g. mixed cell types).

Some other embodiments relate to the use of the product of the present invention for prevention or reduction of the risk of and/or treatment of precancerous conditions of the lung. The term “precancerous conditions in the lung” as used therein refers to a group of cell proliferative disorders of the lung. Cell proliferative disorders of the lung include all forms of cell proliferative disorders affecting lung cells. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, precancerous lung lesion and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

The product of the present invention is also directed at individuals at risk of developing lung cancer. Such risk may be based on the medical or social history of an individual, such as inhalation of tobacco products as it occurs for example in smokers or exposure to asbestos or in non-smokers who breathe in secondhand smoke. Another category of individuals at risk for lung cancer are those harboring genetic mutations predisposing them to lung cancer. Yet another category is individuals who have been exposed to ionizing radiation or chemotherapeutic agents. Finally, another category is individuals with a known cancer at a location other than the lungs that have a propensity to metastasize to the lungs.

In another preferred embodiment, the invention relates to the products described herein for use in the prevention or reduction of the risk of and/or treatment of brain cancers and/or precancerous conditions thereof. The term “brain cancer” as used herein refers to both primary brain tumors and metastatic brain tumors that originate from non-brain cancer cells such as lung cancer cells. Preferably, the term “brain cancer” refers to primary brain tumors.

Primary brain tumors are categorized by the type of tissue in which they first develop. The most common brain tumors are called glioma; they originate in the glial tissue. There are a number of different types of gliomas: for instance, astrocytomas, brain stem gliomas, ependymomas, and oligodendrogliomas.

Other types of primary brain tumors which do not originate from the glial tissue are, for instance, meningiomas, craniopharyngiomas and germinomas.

Inflammation is a complex reaction in vascularized tissues that leads to the accumulation of fluid and leukocytes in extravascular tissues. Closely intertwined with the process of repair, inflammation is fundamentally a protective response. Nevertheless, inflammation and repair may be potentially harmful. Based primarily on its duration, inflammation is divided into acute (of relatively short duration; exudation of fluid, migration of neutrophils) and chronic (of longer duration—more than days; involvement of lymphocytes and macrophages, tissue necrosis).

Inflammation can be induced, among others, by environmental exposure such as smoking. Tobacco smoke also induces pulmonary inflammation (Vlahos et al., Am J Physiol Lung Cell Mol Physiol. 2006; 290:L931-945) and even environmental tobacco smoke inhalation is likely to predispose to acute bronchitis. Furthermore, smoking is a known cause of chronic bronchitis, chronic obstructive pulmonary disease (COPD) and emphysema (Forey et al; BMC Pulmonary Medicine 2011, 11:36). Tobacco smoke-induced pulmonary inflammation contributes to the progressive lung destruction in COPD (Barnes, J Clin Invest. 2008; 118:3546-3556), a condition associated with higher lung cancer risk (Punturieri et al., J Natl Cancer Inst. 2009; 101: 554-559). Indeed, inflammatory mechanisms account for the tumor promoting effect of exposure to tobacco smoke in lung cancer (Takahashi et al. Cancer Cell. 2010; 17: 89).

Several of the compounds described herein have already demonstrated anti-inflammatory properties. For example, we have demonstrated that phosphosulindac V, phospho-aspirin 119 and phospho-ibuprofen 132 strongly inhibit inflammation. e.g., they eliminated or greatly reduced inflammation in animal models of arthritis when applied systemically (Huang L, et al Br J Pharmacol. 2011; 162:1521-33) or topically (Mattheolabakis et al Pharm Res. 2013; 30:1471-82).

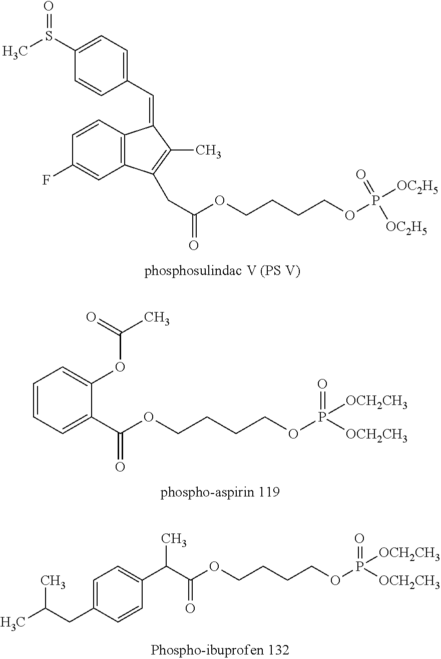 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)c1ccccc1OC(C)=O ; CCOP(=O)(OCC)OCCCCOC(=O)C(C)c1ccc(CC(C)C)cc1]

Their anti-inflammatory effect was based on profound inhibition of the activation of NF-κB, the master regulator of inflammation, and on suppression of inflammatory cytokines and of the pro-inflammatory prostaglandin E2. Furthermore, phospho-valproic acid 134 inhibited pancreatic carcinogenesis in the context of inflammation of the pancreas (chronic pancreatitis) (Mackenzie et al, PLoS One. 2013; 8:e61532).

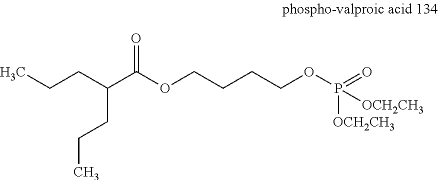 [SMILES: CCCC(CCC)C(=O)OCCCCOP(=O)(OCC)OCC]

In one embodiment, compounds in this invention, especially those with established anti-inflammatory properties, when delivered to the lung will generate a strong anti-inflammatory effect.

In another embodiment, compounds of this invention will generate an anti-inflammatory effect against acute or chronic bronchitis, or against chronic obstructive pulmonary disease or against emphysema or other lung diseases associated with inflammation of the lung, including the upper and lower airways associated with smoking. In another embodiment, one or more anti-inflammatory drugs are combined with one or more anti-cancer drugs and their combination provides an anti-inflammatory effect in the lung and the airways, and an anti-cancer effect. Such anti-inflammatory effect may be provided against inflammation associated with carcinogenesis.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXAMPLES

### Example 1

### Aerosol Administration of Phospho-Sulindac (PS V) Prevented Non-Small Cell Lung Cancer

The following example illustrates the efficacy of PS V administered by inhalation in preventing or reducing the risk of lung cancer.

### Inhalation Exposure System:

The inhalation of PS V was carried out by using the arrangement as described in the U.S. application Ser. No. 13/779,382 and PCT Application No. PCT/US13/28043.

PS V was dissolved in ethanol. PS solution in the baffle was aerosolized with the ultrasonic atomizer. The aerosol passed through an ascending stainless steel column, followed by a reflux column which was maintained at a temperature gradient by a heating tape (82° C.) and a chiller (5° C.) to condense and remove ethanol. PS aerosol exiting the reflux column was then passed through a charcoal column, which served to remove residual traces of ethanol from aerosol before it entered the animal-holding chamber. Experimental animals were held in nose-only air-tight tubes for designated time intervals.

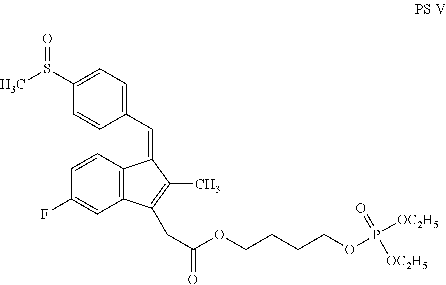 [SMILES: CCOP(=O)(OCC)OCCCCOC(=O)CC1=C(C)/C(=C/c2ccc(S(C)=O)cc2)c2ccc(F)cc21]

### Orthotopic Lung Cancer Model:

BALB/c nude mice (7 weeks old) were divided into control and treatment groups (15 mice/group) and treated following a prevention or reduction of the risk of protocol by administration of either aerosol generated from ethanol (control) or PS V solution (treatment) for one week. The optimized exposure time and dose to mice were 50 mg/mL PS V for 8 min, respectively. On day 1 of week 2, a small incision (˜5 mm) was made to the left side of the chest of anesthetized mice and 1 million GFP-A549 human lung cancer cells (A549 cells expressing green fluorescence protein (GFP) which allows their detection and quantification) were injected into their left lung as described by Doki, Y., et al. (Br. J. Cancer, 79, 7-8, pages 1121-1126, 1999). Inhalation treatment was resumed 2 days post-surgery and continued for 6 weeks when mice were euthanized, and blood and lung tissues were collected. Luminosity of the GFP-A549 tumors was measured and the lungs were weighed.

### Chemopreventive Efficacy:

Two outcomes were used to gauge efficacy, animal survival and tumor size.

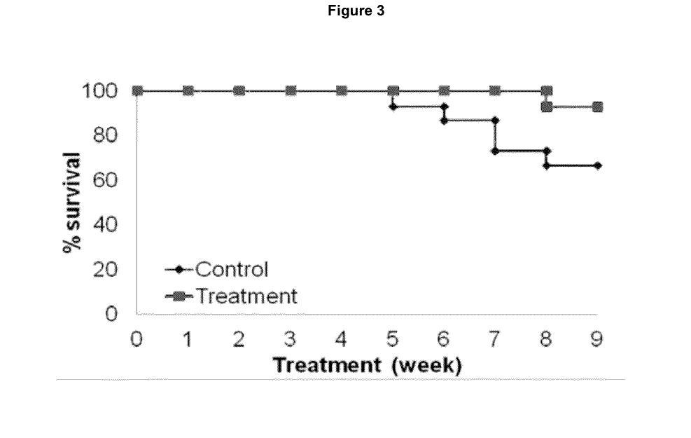

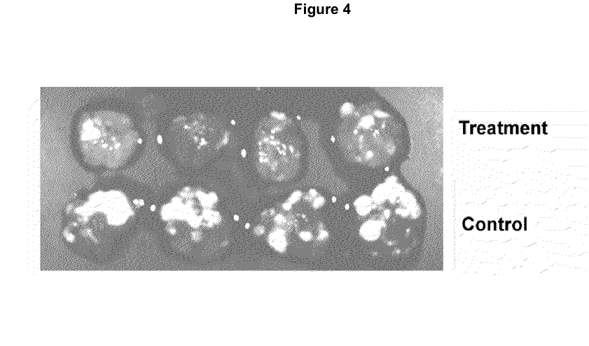

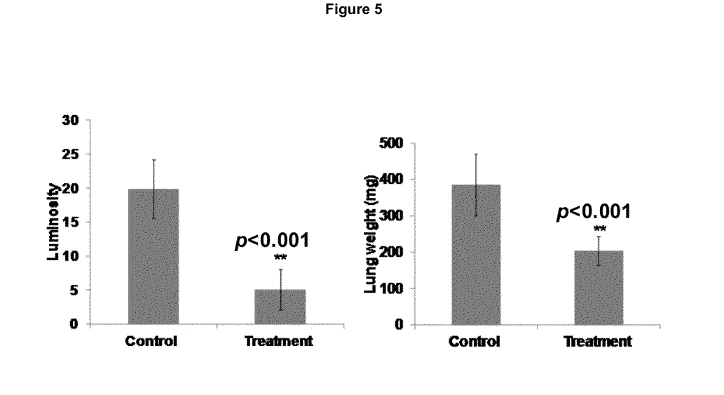

a) Survival: At the end of the study, 40% of the mice in the control group died from the disease while the death rate in the treatment group was less than 10% (p<0.03). The results are illustrated by FIG. 3.
b) Tumor size: At sacrifice, the tumor size was (all values, Mean±SEM) determined a) by luminosity: control=19.85±4.33, treatment=5.05±2.97 (p<0.001). The results are shown in FIG. 4 (upper photograph: after treatment; lower photograph: control group) and FIG. 5 (left hand side); and b) by lung weight: control=385.7±85.2 mg, treatment=204.4±39.4 mg (p<0.001). The results are shown in FIG. 5 (right hand side).

### Example 2

### The Pharmacokinetic Parameters of PS V after Inhalational Administration

PS V was administered to BALB/c nude mice with sulindac, sulindac sulfide XI and sulindac sulfone XII as control. After 8 min of inhalation treatment, BALB/c nude mice were euthanized at various time points. Drug levels were analyzed by HPLC in plasma and lung tissues. These drug levels included PS V as well as sulindac, sulindac sulfide XI and sulindac sulfone XII, the structures of which are shown below,

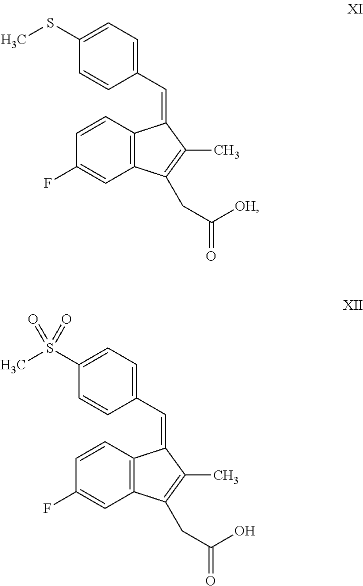 [SMILES: CSc1ccc(/C=C2/C(C)=C(CC(=O)O)c3cc(F)ccc32)cc1 ; CC1=C(CC(=O)O)c2cc(F)ccc2/C1=C\c1ccc(S(C)(=O)=O)cc1]

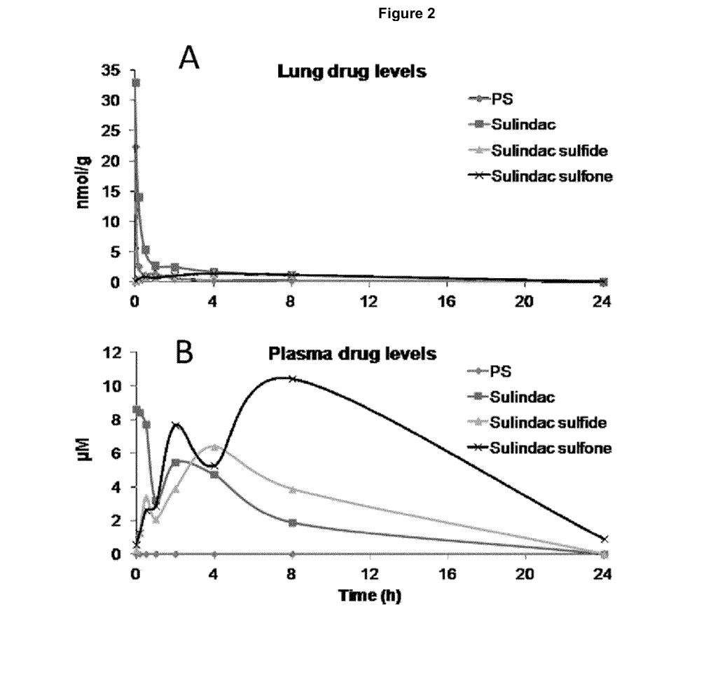

The results are summarized below and are further illustrated in FIG. 2.

TABLE 1

| | Pharmacokinetic parameters in lung | | |
|---|---|---|---|
| | AUC | Cmax, nmol/g | Tmax, h |
| PS V | 7.7 | 22.2 | 0 |
| Sulindac | 30.1 | 32.9 | 0 |
| Sulindac sulfide XI | 18.9 | 1.4 | 4 |
| Sulindac sulfone XII | 57.5 | 4.6 | 8 |

TABLE 2

Pharmacokinetic parameters in plasma

| | AUC | Cmax, μM | Tmax, h |
|---|---|---|---|
| PS V | 0 | 0 | — |
| Sulindac | 49.5 | 8.6 | 0 |
| Sulindac sulfide XI | 66.9 | 6.4 | 4 |
| Sulindac sulfone XII | 142.4 | 10.4 | 8 |

These findings indicate the following: a) inhalation provides intact PS V to the lungs, which is more cytotoxic to human cancer cells than either of its three metabolites, sulindac, sulindac sulfide XI and sulindac sulfone XII; b) oral administration does not provide intact PS V to the lungs, leading only to its three metabolites; and c) there are sufficient concentrations of sulindac and its metabolites in the circulation, and for prolonged periods of time. Sulindac, sulindac sulfide XI and sulindac sulfone XII are established cancer chemopreventive agents and thus, when derived from inhaled PS V, they can prevent smoking/nicotine-related cancers at sites other than the lung.

### Example 3

### Inhalation Delivery of Aerosolized Phospho-Sulindac to the Lungs of Mice LED to Higher Drug Levels than Oral Administration

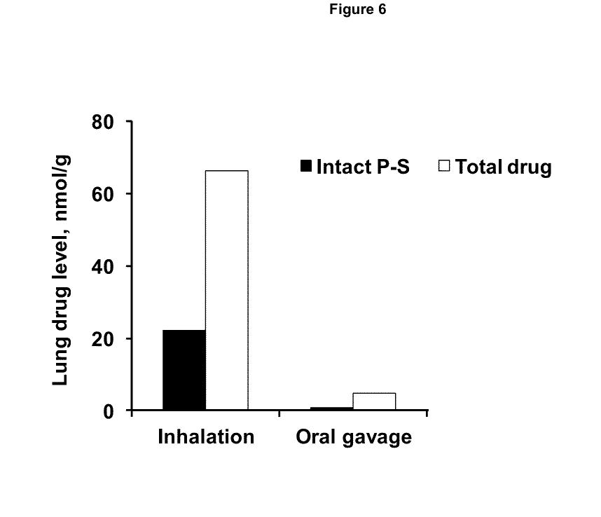

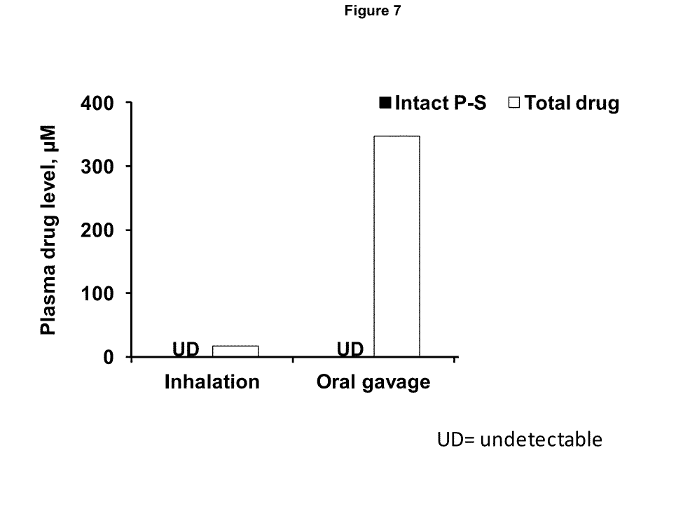

The delivery of aerosolized phospho-sulindac (PS V) to the lungs of mice was evaluated using the same inhalation device as in Example 1 and compared to its oral delivery. The PS V doses were: inhalational=6.5 mg/kg body weight; oral=150 mg/kg body weight. The oral dose was 23 times higher than the inhalational dose. The level of PS V in the lungs and plasma after inhalation vs. after oral gavage are shown in FIGS. 6 and 7, respectively.

### Lungs:

PS levels: The aerosol-exposure system delivered a high level of intact PS V to the lungs of mice (>20 nmol/g); while there were only trace levels of intact PS V (<2 nmol/g) by oral administration. Total drug levels: It represents the total level of PS V plus its metabolites. The main metabolites of PS V are sulindac, sulindac sulfide XI and sulindac sulfone XII; at least the first two can cause gastrointestinal and renal side effects. The levels achieved by inhalation were significantly higher compared to those by oral administration.

### Plasma:

PS levels: undetectable. Total drug levels after inhalation treatment (17 μM) were lower than after oral (348 μM) administration. Thus, inhalation delivery led to blood levels of sulindac that can be chemopreventive for various non-lung cancers, but which were not particularly high as not to have significant potential toxicity.

Thus, PS V can be effectively delivered to lung cells by inhalation of a mixture of tobacco smoke with aerosolized PS V.

### Example 4

### Curcumin Enhanced the Lung Cancer Chemopreventive Efficacy of Phospho-Sulindac V

Curcumin, the principal bioactive component in turmeric, exhibits anti-tumorigenic activities. In pre-clinical models of lung cancer, however, curcumin as a single agent has demonstrated poor efficacy (<30%). The present example demonstrates that curcumin potentiates the anti-cancer efficacy of PS V in the A549 human non-small cell lung cancer (NSCLC) cells, and that such a combination synergistically inhibited the growth of A549 xenografts in mice. These findings suggest that PS V in combination with curcumin is a promising combination therapy for the prevention or reduction of the risk of NSCLC.

Polymeric nanoparticles of poly(ε-caprolactone) (11000)-polyethylene glycol (5000) with entrapped curcumin were prepared according to the nanopercipitation-solvent displacement method. Four groups of female nude mice (n=6 per group) at 7-8 weeks of age, were pre-treated for three days with 1) vehicle; 2) PS V 200 mg/kg/d; 3) curcumin 500 mg/kg/d; and 4) PS V 200 mg/kg/d plus curcumin 500 mg/kg/d. Then, the mice were inoculated subcutaneously on both flanks with A459 cells (2×10⁶ each). The treatment was resumed one day after tumor implantation and continued daily until the end of the study.

PS V alone produced a small inhibition of tumor growth that was statistically significant on days 12-22 after tumor implantation; whereas curcumin was ineffective for the duration of the study. On the other hand, PS V in combination with curcumin synergistically inhibited the growth of A549 xenografts, and the effect was statistically significant (p<0.05) beginning on day 12 until the end of the study (day 36). At the end of the study, the average tumor volume of each group was as follows: control: 521±76 mm³; PS V: 419±36 mm³; curcumin: 599±98 mm³; PS V plus curcumin: 290±54 mm³. This corresponds to a reduction in tumor volume of 19.6% and 44.3% for PS V and PS V plus curcumin, respectively. In terms of tumor weight, a reduction was observed in the PS V (27%, p=0.06) and the PS V plus curcumin (51%, p<0.01) groups, but not in the curcumin-treated group. Of note, PS V plus curcumin treatment was significantly more effective than PS V or curcumin alone (p<0.05).

### Example 5

### Transdermal Patch Containing Nicotine and an Anti-Cancer Agent

The transdermal patch of the present invention can be manufactured analogously to the procedure disclosed in U.S. Pat. No. 7,387,788.

Ethanol, propylene glycol, diethylene glycol monoethyl ether (and myristyl alcohol) are weighed and added successively. The mixture is homogenized using mechanical mixing. The resulting organic solution is clear and homogeneous. Nicotine hydrogen tartrate is added to 85-90% of the total amount of water and mixed until the solution is homogenized. Then the resulting aqueous solution is added to the organic solution, followed by an anti-cancer agent, such as phospho-sulindac V (PS V) and mixed until homogenization of the solution is achieved. The resulting solution is clear and homogeneous. Then triethanolamine (typically about 50 wt.-% aqueous solution) is added and the solution mixed until the solution becomes homogeneous. The resulting solution is clear and homogeneous with a pH, for example, of about 6. When the pH is within the desired specification range water is added to the solution to obtain the desired weight percents (wt.-%) of the components and the pH of the final solution is measured. If the pH is below the desired pH (e.g. about pH 5.5), further triethanolamine solution is added and the pH of the final solution is re-measured. Typically, total triethanolamine amount does not exceed 5 wt.-%.

The composition of exemplary formulations 5.1-5.3 is summarized in Table 3.

TABLE 3

Composition (wt.-%)

| | Formula- | Formula- | Formula- |
|---|---|---|---|
| Component | tion 5.1 | tion 5.2 | tion 5.3 |
| Nicotine tartrate | 2.85 | 2.85 | 2.85 |
| Absolute ethanol | 40.00 | 40.00 | 40.00 |
| Phospho-sulindac V | 5.00 | 2.00 | 1.00 |
| Diethylene glycol | 5.00 | 5.00 | 5.00 |
| monoethyl ether | | | |
| Propylene glycol | 15.00 | 25.00 | 25.00 |
| Myristyl alcohol | 1.00 | 1.00 | 0.00 |
| Hydroxypropylcellulose | 1.50 | 1.50 | 1.50 |
| (Klucel HF) | | | |
| Triethanolamine | 5.07 | 3.52 | 4.00 |
| (50% w/w) | | | |
| Purified water | 24.58 | 19.13 | 20.65 |

### Example 6

### Cigarette Containing an Anti-Cancer Agent

The cigarette according to the present invention can be manufactured analogously to the procedure disclosed in US 2011/061667.

To 5 g of powdery agar (Wako Pure Chemical Industries, Ltd.) 100 ml of water is added, and the mixture is heated in a thermostat bath at 80° C. to dissolve agar. 25 g of l-menthol, 1.5 g fluorouracil and 2 ml of a 5 wt.-% aqueous solution of lecithin as an emulsifier are added thereto, and the mixture is sufficiently emulsified by means of a homogenizer. This emulsified slurry is cast on a substrate into a sheet form, which is dried in a forced air circulation dryer of 40° C. for one week. At this time, the emulsified state of the mixture is kept while the material is being dried.

The flavor-containing material for cigarette is blended in 5% by weight ratio to cut tobacco, and cigarettes with a tar value designed to about 10 mg are produced. The cigarettes may be optionally fitted with a plain filter.

### Example 7

### Chewing Gum Containing Nicotine and an Anti-Cancer Agent

The chewing gum according to the present invention can be manufactured analogously to the procedure disclosed in US 2010/0130562. An example of a chewing gum composition is shown in Table 4.

TABLE 4

| Component | Composition (wt.-%) |
|---|---|
| Nicotine Polacrilex (18%) | 2.55 |
| Phospho-ibuprofen I | 2.00 |
| DREYCO ® gum base | 65.99 |
| Sorbitol | 22.1 |
| Fruit mint flavor | 3.8 |
| Sodium carbonate | 2.00 |
| Sodium bicarbonate | 1.00 |
| Acesulfame potassium | 0.25 |
| L-menthol | 0.25 |
| D&C Yellow 10 and Brown Lakes | 0.06 |

The composition is prepared by adding 1359.7 g of DREYCO® gum base to a jacketed high shear mixer. The gum base is heated to about 6° C. and 50.9 g of Nicotine Polacrilex, 2.00 g of phospho-ibuprofen I, 442 g of sorbitol, 76 g of fruit mint flavor with ethanol as a carrier, 40 g of sodium carbonate, 20 g of sodium bicarbonate, 5.0 g of acesulfame potassium, 5.0 g of L-menthol and 0.8 g of D&C Yellow 10 and Brown Lakes are added. After the ingredients are mixed, the mixture is cooled to approximately 38° C. and removed from the mixer and then rolling and scoring process are performed to produce individual gum pieces. The gums are packaged into high density polyethylene bottles that are sealed and capped.

### Example 8

### Aerosolization of Anticancer Compounds

The proposed human applications of the methods claimed herein require the conversion of the anticancer drug into an inhalable form. Anticancer drugs are usually solids or liquids, requiring a transition to the gas phase. The relevant terminology can at times be confusing. Aerosolization is the process of converting some physical substance into the form of particles small and light enough to be carried on the air i.e. into an aerosol. Sublimation, a phase transition which may occur with our tested compounds, is the (endothermic) transition of a substance directly from the solid to the gas phase without passing through an intermediate liquid phase.

The experiment evaluated two approaches: a) mixing the anticancer drug with tobacco; and b) using a device that allows aerosolization (most likely via sublimation) of the test drug. We studied several anticancer agents, including phospho-ibuprofen amide IV, PS V, and PS amide 106.

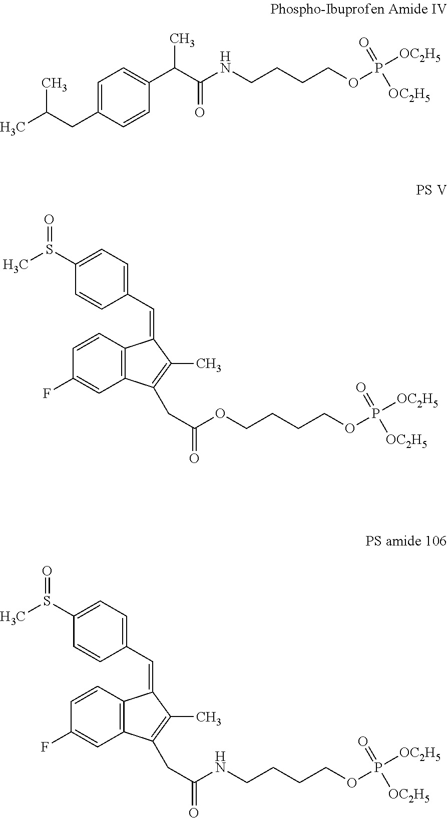 [SMILES: CCOP(=O)(OCC)OCCCCNC(=O)C(C)c1ccc(CC(C)C)cc1 ; CCOP(=O)(OCC)OCCCCOC(=O)CC1=C(C)/C(=C/c2ccc(S(C)=O)cc2)c2ccc(F)cc21 ; CCOP(=O)(OCC)OCCCCNC(=O)CC1=C(C)/C(=C/c2ccc(S(C)=O)cc2)c2ccc(F)cc21]

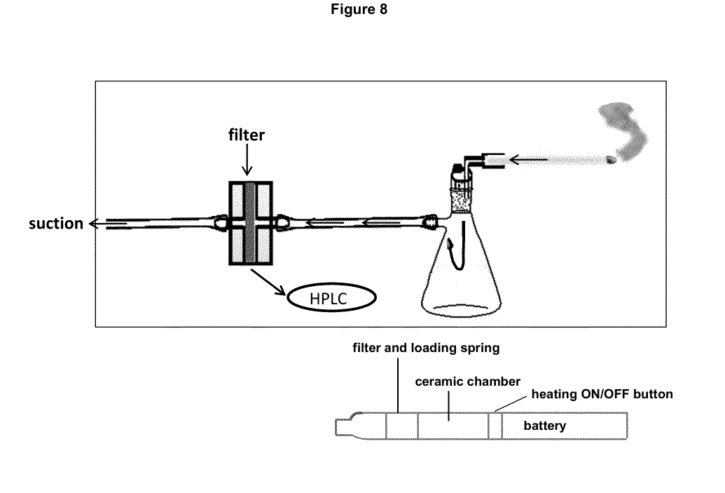

We used an experimental system that recapitulates the essential features of the act of smoking (FIG. 8). In this apparatus, smoke from the lit cigarette was drawn through the tubing of the apparatus by external suction. As shown, smoke encountered a 50 mm filter placed in the filter holder (Whatman 10461100 Polysulfone FP 050/0 Filter Holder). The aerosolized test drug was deposited onto this filter. At the conclusion of the study, the filter was removed and the deposited test drug was extracted with acetonitrile and subjected to HPLC analysis, as already described. In a different iteration of this approach we substituted in this apparatus the cigarette with the device depicted in FIG. 8 (lower panel).

FIG. 8. depicts the apparatus for the aerosolization of anticancer compounds. Upper panel: The test compound is added to the cigarette. Lower panel: The device used to evaluate the aerosolization of the test compound, which is placed in the heating ceramic chamber (no tobacco). When the system was turned on, the temperature in the heating chamber reached ˜200° C. Heating energy was provided by the battery through a resistance wire inside the heating chamber. Unimpeded flow of the gas phase in the ceramic chamber was allowed by its design; the arrows depict such flow.

We evaluated the aerosolization of phospho-ibuprofen 132, phospho-ibuprofen amide IV and the kinase inhibitor erlotinib, when each compound was added to smoking tobacco.

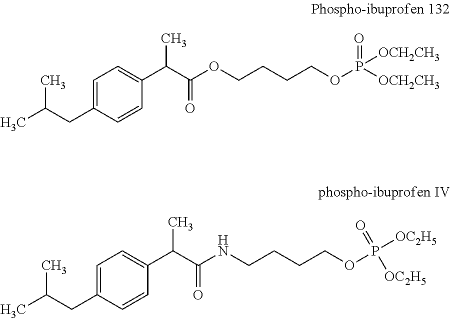 [SMILES: CCOP(=O)(OCC)OCCCCOC(=O)C(C)c1ccc(CC(C)C)cc1 ; CCOP(=O)(OCC)OCCCCNC(=O)C(C)c1ccc(CC(C)C)cc1]

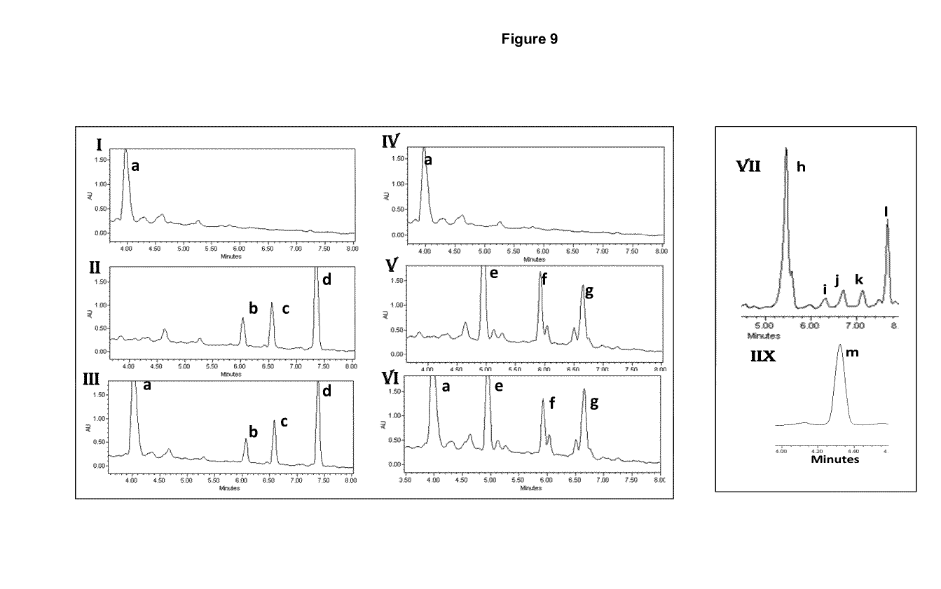

Each of Phospho-ibuprofen 132 and phopho-ibuprofen amide IV was also mixed with erlotinib and aerosolized. As shown in FIG. 9 (chromatograms I-VI), all three compounds were aerosolized successfully. Erlotinib generated a single peak. The other two compounds generated additional peaks; which were also observed as a result of the in vivo metabolism of these compounds. Remarkably, the combination of two compounds had no adverse effect on the aerosolization of each member of the pairs tested. Aerosolization of PIA occurred only when the cigarette was lit, indicating that the energy from the burning tobacco (>1,000 C) was used to likely sublimate PIA. Additional compounds gave similar results.

FIG. 9 depicts chromatograms from aerosolized test compounds. In particular, in the left panel, all compounds were mixed with tobacco and the resultant cigarettes were treated as in FIG. 8. Chromatograms (I) and (V) were obtained from cigarettes with erlotinib: erlotinib, which appears as the single peak a. Chromatogram (II) was obtained from cigarettes with phospho-ibuprofen 132. Peaks: b, ibuprofen; c, dephosphorylated phospho-ibuprofen 132; Peaks: d, phospho-ibuprofen 132. Chromatogram (III), was obtained from cigarettes with a mixture of erlotinib and phospho-ibuprofen 132, generating the same peaks as each one individually. Chromatogram (V) was obtained from cigarettes with phospho-ibuprofen amide. IV (PIA). Peaks: e, 1,3 dihydroxy PIA, f, PIA, g, dephosphorylated PIA. In the right panel, two compounds, PS V and phospho-sulindac amide 106, were aerosolized using the device shown in FIG. 8 (not mixed with tobacco). Chromatogram (VII) was obtained from PS V. Peaks; (h), PS V; (i), unknown; (j), sulindac sulfide; (k), unknown; (I), phospho-sulindac (PS) V sulifide. Chromatogram (IIX) was obtained from phospho-sulindac amide 106. Peak: (m), phospho-sulindac 106.

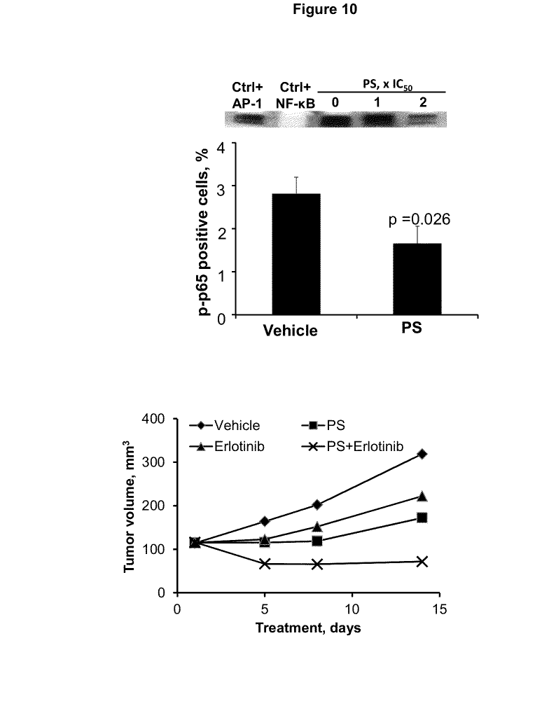

In a parallel study, we evaluated the anti-lung cancer activity of PS V and erlotinib each alone and combined with each other. TO this end, we inoculated A549 human lung cancer cells subcutaneously into nude mice, following standard protocols. When tumors reached an average volume of ˜120 mm³, we commenced treatment with PS V 80 mg/kg ip or erlotinib 75 mg/kg po or both at the same doses; all were administered 6 days/week. Tumor volume was monitored as shown in FIG. 10. FIG. 10 provide the effect of PSV and erlotinib on lung cancer. Erlotinib administered in combination with PSV regresses human lung cancer xenografts in nude mice, in contrast to each compound alone that partially inhibited tumor growth. By day 14, compared to controls, PS V had reduced tumor volume by 49%, erlotinib by 74%, and PS V plus erlotinib by 124% (In these calculations the starting tumor volume has been subtracted). It is remarkable that PS V plus erlotinib regressed tumors by 40% from their starting tumor volume.

### Example 9

### Inhibition of NF-κB in Lung Cancer by Phosphosulindac V

We evaluated the effect of PS V on the activation of NF-κB, the master regulator of inflammation, in cultured A549 human lung cancer cells as well as in human lung cancer xenografts from the same cells.

In cultured A549 cells, PS V at a concentration 1-2× its IC₅₀ concentration for 6 h. suppressed NF-κB activation, assessed by Electrophoretic Mobility Shift Assay (EMSA). We established orthotopic xenografts in nude mice as described in Example 1. Following treatment of the mice with PS V in a similar manner, lung tissue was evaluated by immunohistochemistry, following standard protocols. We used an antibody that assesses activation of NF-κB by phosphorylation (Cell Signaling, Danvers, Mass.). FIG. 10 provides inhibitory effect of PS V on the activation of NF-κB. The results shown in FIG. 10 indicate that PS V suppresses the activation of NF-κB.

### Example 10

### Device for the Delivery of Drugs During Smoking

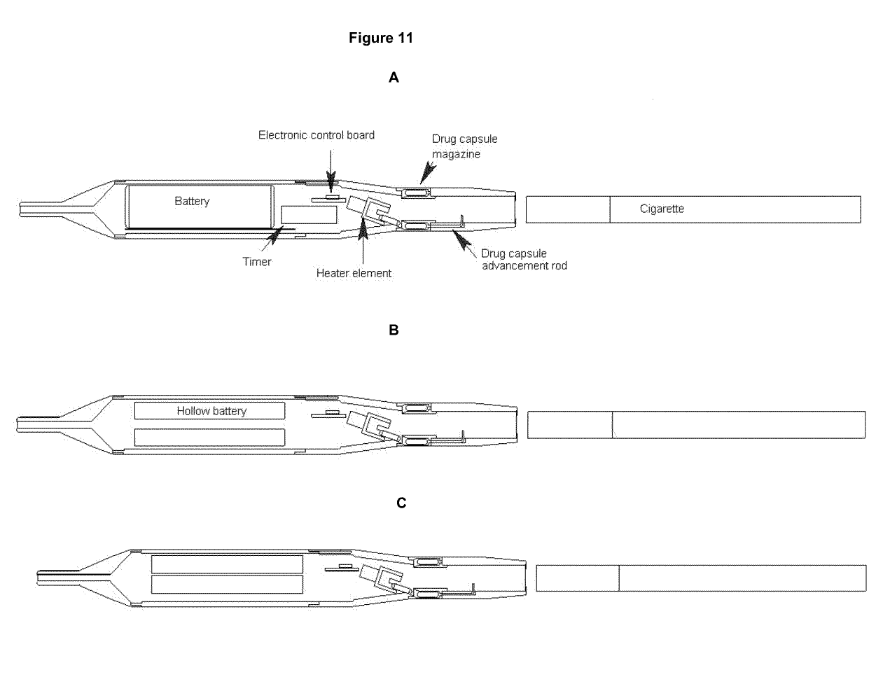

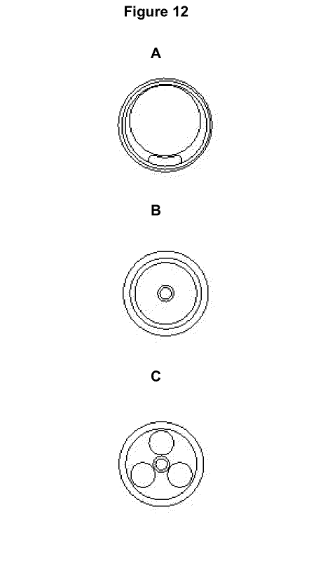

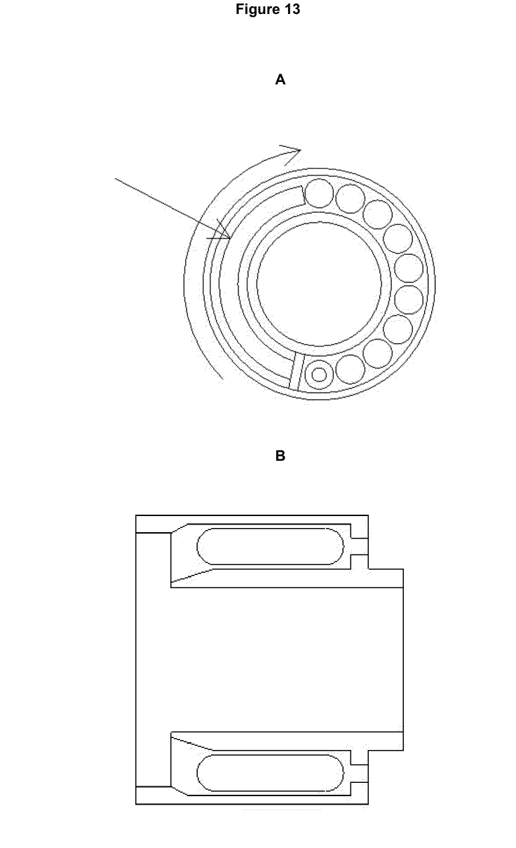

FIG. 11A shows a cigarette holder and agent dispensing device in side cross-section. The device has a cigarette receiving chamber on its right side. When a cigarette is slid to the left in the cigarette receiving chamber, its left end will push against a spring loaded drug containing solid carriers advancement rod to drive it to the left which will push the capsule in the six o'clock position into a heater section. The drug can be in a formulation suitable for this mode of transition; for example, but not limited to, the following: pellet, capsule, tablet, microspheres, granules, micro/nanoparticles. This action will also activate the heater to start heating the drug to vaporize it for a certain period of time, to vaporize the entire capsule. When the cigarette is later retracted, the spring loaded drug capsule advancement rod will return to the position shown in FIG. 12A. The drug loading magazine will be described below in connection with FIGS. 13A and 13B.

When the drug is loaded into the heater section, the electronic control board will cause heating of the capsule and its vaporization. Combined with the smoke from a burning cigarette, the vaporized drug will be provided to exit left of the device at the mouthpiece.

The device includes a timer which has a time-of-day clock. The control board includes a display which is visible through a transparent window outside the device. The display can display time of day, how many capsules have been consumed during a prior time period such as earlier in the day. In this way closing either daily or of some other time period can be monitored. In some cases, a maximum amount of drug should not be exceeded on a daily or other periodic basis. If that limit is reached, the electronic control board could prevent activation of the heater element.

The device includes an electronic control board for controlling the maximum amount of drug that can be dispensed during a predetermined period of time.

The device includes an electronic control board for controlling the duration of the operation of the heating element or its heating period.

The device is separable at the location of the location of the drug magazine to enable replacement of the magazine. A clear window can surround all or part of the magazine to enable viewing of the remaining capsules. The display could also provide a count of capsules consumed and remaining.

The drug is formulated to result in little or no residue, so that little or no maintenance would be required. When the magazine is replaced, a user could check for any residue and shake out if any exists.

The power for the electronic control board can be a battery of AAA type, for example, or some different size, and possibly smaller. The mouthpiece end is removable to enable battery replacement. The control board and display could also monitor energy consumption from the battery, or battery voltage reading and provide display output indicating battery status, such as whether the battery needs to be replaced. A blinking LED could also provide such a warning signal.

The electronic control board has a memory which can store information such as time of day each drug was administered, number of drug capsules consumed from the present magazine, number of drug capsules remaining for use in the magazine, type of drug to be dispensed and its preferred or operative vaporization temperature, and the like.

The electronic control board will control the heater, and through appropriate sensing and control circuitry control the power provided to the heater to reach and maintain the correct vaporization temperature of the drug.

FIGS. 11B and 12B show a device like that of FIGS. 11A and 12A except that the battery is hollow, which provides a flow passageway through the center of the battery.

FIGS. 11C and 12C show a device like that of FIGS. 11A and 12A except that the battery power is arranged as a plurality of batteries, in this case three, but the number can be any plurality. The spacing between the batteries provides a flow passageway.

FIG. 13A shows a drug capsule magazine or cartridge usable in the device of FIGS. 11A-11C. The magazine has a arcuate channel around at least part of its outer region. The channel receives a plurality of capsules, ten of which are shown. A spring loaded plunger biases all of the capsules clockwise, so that the capsules are available for ejection at the lower position.

FIG. 13B shows the capsule cartridge in a cross-section side view. Two capsules, in the lowest and highest positions (approximately the 6 and 12 o'clock positions) are shown, but other capsules not shown are between these two positions.

The magazines, because they contain drugs, can be dispersed by a pharmacy. The device or holder can be sold through other channels.

The devices can be designed to have different variety of magazine slots to accept different shaped magazines depending on the type of drug in the magazine. The magazine can have indicia which are read by the control board so the control board knows the time and temperature to engage the heater for proper and full vaporization of the particular drug.

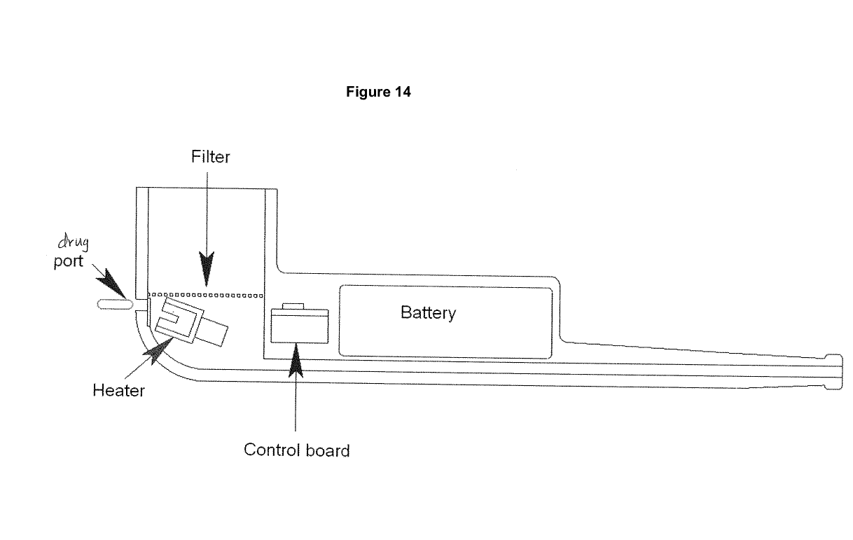

FIG. 14 is a cross-sectional view of a pipe adapted to dispense smoke from burning tobacco and vaporized drug. The pipe includes a tobacco chamber having a filter at the bottom. Below the filter is a heater which can receive a drug capsule inserted into the pipe through a drug port. The drug port has a door which is biased in the closed position as shown but opens when a drug capsule is pushed through the port. A control board and battery provide power and are connected to provide controlled energy to the heater to heat the drug to the operative vaporization temperature to vaporize the drug. Similar to the embodiments shown in FIGS. 11A-11C, the pipe can include a drug magazine, display and other features.

The device can receive the drug in the form of, among others, powder, granules, microspheres, nano/microparticles that are deposited into the heating chamber directly through a dedicated opening.

### Other Embodiments

All publications, patent applications, and patents mentioned in this specification are herein incorporated by reference.

Various modifications and variations of the described compositions, methods, and kits of the invention will be apparent to those skilled in the art without departing from the scope and spirit of the invention. Although the invention has been described in connection with specific embodiments, it will be understood that the invention as claimed should not be unduly limited to such specific embodiments. Indeed, various modifications of the described modes for carrying out the invention that are obvious to those skilled in the fields of molecular biology, medicine, immunology, pharmacology, virology, or related fields are intended to be within the scope of the invention.

We claim:

1. A composition comprising
a nicotine-containing material; and
an agent comprising at least one of an anti-cancer agent, an anti-inflammatory agent, or a combination thereof.

2. The composition according to claim 1, wherein the agent comprises a phospho-NSAID.

3. The composition according to claim 1, wherein the agent comprises a compound selected from the group consisting of

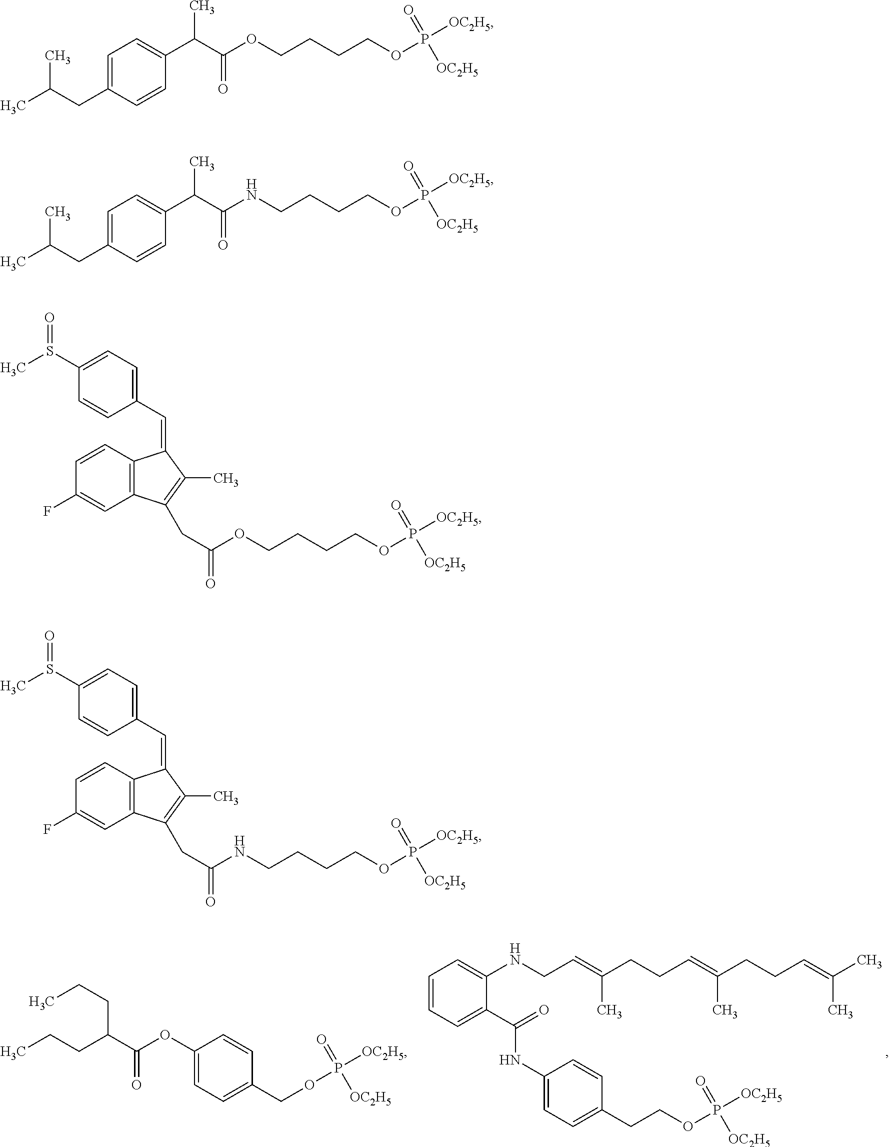 [SMILES: CCOP(=O)(OCC)OCCCCOC(=O)C(C)c1ccc(CC(C)C)cc1 ; CCOP(=O)(OCC)OCCCCNC(=O)C(C)c1ccc(CC(C)C)cc1 ; CCOP(=O)(OCC)OCCCCOC(=O)CC1=C(C)/C(=C/c2ccc(S(C)=O)cc2)c2ccc(F)cc21 ; CCOP(=O)(OCC)OCCCCNC(=O)CC1=C(C)/C(=C/c2ccc(S(C)=O)cc2)c2ccc(F)cc21 ; CCCC(CCC)C(=O)Oc1ccc(COP(=O)(OCC)OCC)cc1 ; CCOP(=O)(OCC)OCCc1ccc(NC(=O)c2ccccc2NC/C=C(\C)CC/C=C(\C)CCC=C(C)C)cc1]

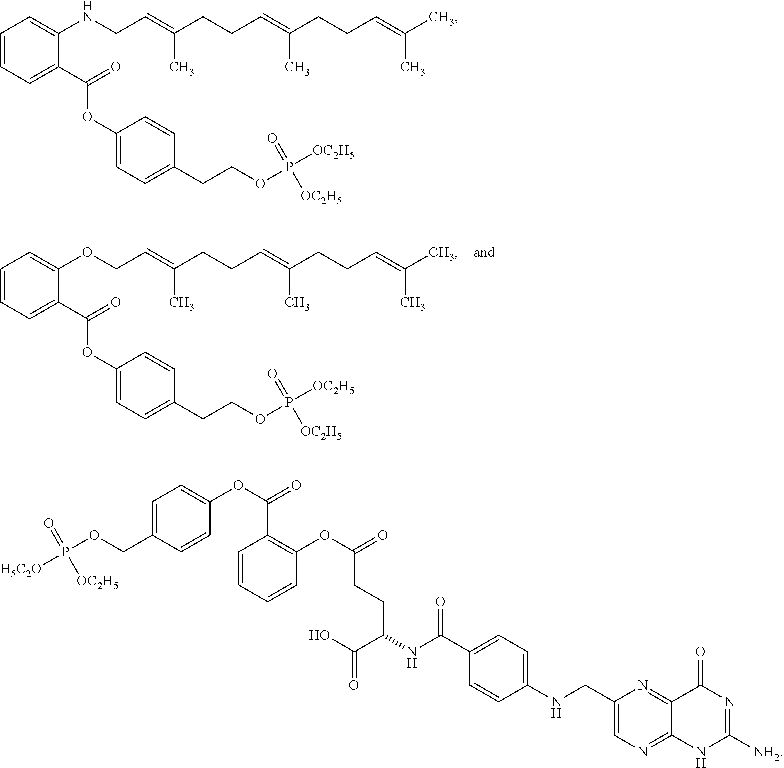

4. The composition according to claim 1, wherein the agent comprises a compound selected from the group consisting of

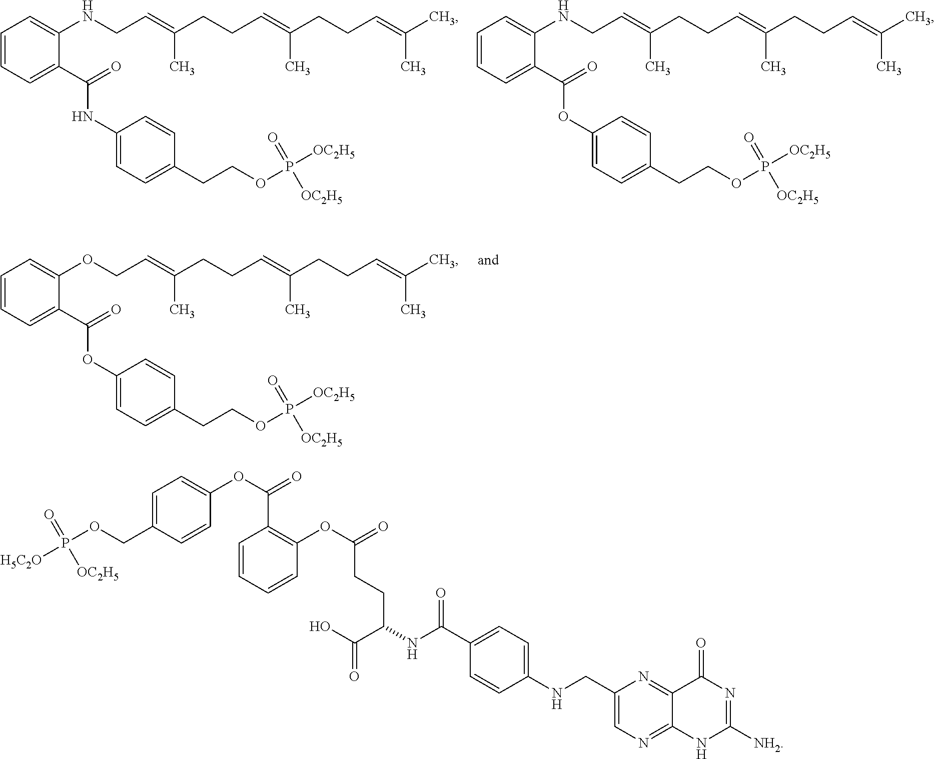

5. The composition according to claim 1, wherein the agent comprises an anti-cancer agent and an anti-inflammatory agent.

6. The composition according to claim 1, wherein the agent comprises at least two anti-cancer agents.

7. The composition according to claim 1, wherein the agent comprises at least two anti-inflammatory agents.

8. The composition according to claim 5, wherein the agent comprises phospho-NSAID and curcumin.

9. The composition according to claim 1, wherein the nicotine-containing material is tobacco leaf.

10. The composition according to claim 1, wherein the composition contains nicotine and the agent in the ratio of from 1000:1 to 1:10 (wt:wt).

11. The composition according to claim 1, wherein the composition is provided in the form of a smoking device selected from the group consisting of cigarette, cigar and smoking pipe.

12. The composition according to claim 11, wherein the smoking device includes a unit which renders the agent suitable for inhalation.

13. The composition according to claim 1, wherein the composition is a smoking cessation product.

14. The composition according to claim 1, wherein the composition is in the form of a transdermal patch.

15. The composition according to claim 11, wherein the smoking device is an inhalation device.

16. The composition according to claim 15, wherein the device is an electronic cigarette.

17. The composition according to claim 1, wherein the composition is an orally applied product.

18. The composition according to claim 17, wherein the product is a smokeless tobacco product.

19. A method for preventing or reducing the risk of inflammation comprising:
administering an effective amount of an anti-inflammatory agent, and a source of nicotine to a subject.

20. The method of claim 19 wherein the anti-inflammatory agent is administered simultaneously with the source of nicotine.

21. The method of claim 19 wherein the anti-inflammatory agent is administered sequentially with the source of nicotine.

22. The method of claim 19 wherein the step of administration is by inhalation of the anti-inflammatory agent and tobacco smoke.

23. A method for treating inflammation, comprising:
administering an effective amount of an anti-inflammatory agent, and a source of nicotine to a subject in need thereof.

24. The method of claim 23 wherein the anti-inflammatory agent is administered simultaneously with the source of nicotine.

25. The method of claim 23 wherein the anti-inflammatory agent is administered sequentially with the source of nicotine.

26. The method of claim 23 wherein the step of administration is by inhalation of the anti-inflammatory agent and tobacco smoke.

27. A method for treating inflammatory conditions, comprising:
administering an effective amount of an anti-inflammatory agent, and a source of nicotine to a subject in need thereof.

28. The method of claim 27 wherein the anti-inflammatory agent is administered simultaneously with the source of nicotine.

29. The method of claim 27 wherein the anti-inflammatory agent is administered sequentially with the source of nicotine.

30. The method of claim 2275 wherein the step of administration is by inhalation of the anti-inflammatory agent and tobacco smoke.

31. A method for preventing or reducing the risk of cancer comprising:
administering an effective amount of an anti-cancer agent, and a source of nicotine to a subject.

32. The method of claim 31 wherein the anti-cancer agent is administered simultaneously with the source of nicotine.

33. The method of claim 31 wherein the anti-cancer agent is administered sequentially with the source of nicotine.

34. The method of claim 31 wherein the step of administration is by inhalation of the anti-cancer agent and tobacco smoke.

35. A method for treating a cancer, comprising:
administrating an effective amount of an anti-cancer, and a source of nicotine to a subject in need thereof.

36. The method of claim 35 wherein the anti-cancer agent is administered simultaneously with the source of nicotine.

37. The method of claim 35 wherein the anti-cancer agent is administered sequentially with the source of nicotine.

38. The method of claim 35 wherein the step of administration is by inhalation of the anti-cancer agent and tobacco smoke.

39. A method for treating lung cancer, comprising:
administering an effective amount of an anti-cancer agent, and a source of nicotine to a subject in need thereof.

40. The method of claim 39 wherein the anti-cancer agent is administered simultaneously with the source of nicotine.

41. The method of claim 39 wherein the anti-cancer agent is administered sequentially with the source of nicotine.

42. The method of claim 39 wherein the step of administration is by inhalation of the anti-cancer agent and tobacco smoke.

43. A dispensing device for dispensing nicotine and a vaporized drug, comprising:
a tube having a first end adapted to receive a cigarette;
said tube having a heater for accepting a drug to be vaporized; and
a second end adapted to emit cigarette smoke and vaporized drug when suction force is applied by a user.

44. The dispensing device according to claim 43, further comprising a drug magazine which holds a plurality of drug containing solid carriers formulated as a capsule, tablet or pellet.

45. The dispensing device according to claim 43, wherein the drug containing solid carriers have a cylindrical shape.

46. The dispensing device according to claim 43, wherein the device is separable along its length to permit removal and replacement of the drug magazine.

47. The dispensing device according to claim 43, wherein the drug magazine is in the form of a circular ring, and wherein the plurality of drug containing solid carriers are arranged in a circumferential slot arranged near the outside of the circular ring.

48. The dispensing device according to claim 43, wherein the drug magazine includes a biasing element to bias the drug containing solid carriers toward a select location along the circular ring.

49. The dispensing device according to claim 43, wherein the dispensing device further includes an electronic control board for controlling the operation of the heater.

50. The dispensing device according to claim 43, wherein the dispensing device includes a battery chamber along its length for receiving a battery used to energize the electronic control board and heater.

51. The dispensing device according to claim 43, wherein the battery is in the form of a single cylinder, and wherein the device defines a hollow passageway alongside the battery for passage of cigarette smoke and vaporized drug.

52. The dispensing device according to claim 43, wherein the dispensing device further includes an electronic control board for controlling the maximum amount of drug that can be dispensed during a period of time.

53. The dispensing device according to claim 43, wherein the device includes a loading mechanism, engaged when the tube receives a cigarette, which loads a drug containing solid carriers into the heater.

54. The dispensing device according to claim 43, wherein the battery is in the form of a plurality of cylinders, and wherein the cylinders are spaced to provide hollow passageways for passage of cigarette smoke and vaporized drug.

55. The dispensing device according to claim 43, wherein the battery is in the form of a cylinder with a hollow core, to define a hollow passageway for passage of cigarette smoke and vaporized drug.

56. The dispensing device according to claim 43, wherein the device includes a transparent window surrounding the drug magazine to see the drug containing solid carriers from outside the device.

57. The dispensing device according to claim 43, wherein the device includes a display for displaying indicia indicating the number of drug containing solid carriers used from the magazine.

58. The dispensing device according to claim 43, wherein the device includes a display for displaying indicia indicating the number of drug containing solid carriers in the magazine.

59. The dispensing device according to claim 43, wherein the device includes an electric timer.

60. The dispensing device according to claim 59, wherein the timer has a time of day clock.

61. The dispensing device according to claim 59, wherein the timer detects the time between successive dispensing of the drug.

62. A drug magazine for holding a plurality of drug containing solid carriers comprising:
a circular ring defining a circumferential slot arranged near the outside of the circular ring, wherein the slot is adapted to receive a plurality of drug containing solid carriers.

63. The magazine of claim 62, further including a biasing element which biases the drug containing solid carriers to a selected location along the circular ring for dispensing.

64. The magazine of claim 62, wherein the biasing element is an elongated spring held in compression in the circular ring.

65. A pipe for dispensing nicotine and a vaporized drug, comprising:
a tobacco receiving chamber for receiving tobacco to be combusted;
a heater for receiving a drug and heating the drug to a temperature for vaporizing the drug; and
a pipe stem ending in a mouthpiece and having a passageway for passage of smoke from combusted tobacco and vaporized drug.

66. The pipe of claim 65, further including a port in the chamber for inserting a drug into the heater.

67. The pipe of claim 65, wherein the port has a door.

68. The pipe of claim 65, wherein the pipe includes a control board and battery for providing and controlling energy to the heater to vaporize the drug.

69. The pipe of claim 65, wherein the pipe includes a control board for providing in a visual display the amount of drug used in a given period of time.

70. The pipe of claim 65, wherein the pipe includes a control board for preventing the addition of drug to the pipe beyond a given amount for a given period of time.
